(19) United States
(12) Patent Application Publication
(10) Pub. No.: US 2009/0269773 A1
(43) Pub. Date: Oct. 29, 2009

(54) METHODS OF DETERMINING THE HEALTH STATUS OF AN INDIVIDUAL

(75) Inventors: Wendy J. Fantl, San Francisco, CA (US); Helen Francis-Lang, Sticklepath Nr (GB); Alleen C. Cohen, Palo Alto, CA (US); Garry P. Nolan, South San Frncisco, CA (US); Malcol Frncis-Lang, Sticklepath Nr (GB)

Correspondence Address: WILSON, SONSINI, GOODRICH & ROSATI / NODALITY, INC, 650 Page Mill Road, Palo Alto, CA, 94304-1050, US

(73) Assignee: Nodality, Inc. A Delaware Corporation

(21) Appl. No.: 12/432,720

(22) Filed: Apr. 29, 2009

Related U.S. Application Data
(60) Provisional application No. 61/048,886, filed on Apr. 29, 2008.

(51) Int. Cl. C12Q 1/68; C12Q 1/02; G01N 33/574; C12Q 1/37; C12Q 1/48; C12Q 1/42; C12Q 1/26; G01N 33/53; G06F 19/00

(52) U.S. Cl. 435/6; 435/29; 435/7.23; 435/23; 435/15; 435/21; 435/25; 435/7.92; 702/19

(57) ABSTRACT

Methods of determining health status based on analysis of single cells in a sample or set of samples from an individual are described.

### CROSS-REFERENCE

This application claims the benefit of the filing date of U.S. Ser. No. 61,048,657 filed Apr. 29, 2008, this provisional application is hereby expressly incorporated by reference in their entirety.

### BACKGROUND OF THE INVENTION

Even though there have been great gains in knowledge over the past several decades in the fields of genetics and cellular and molecular biology, this expansion of knowledge has not translated into commensurate advances in the diagnosis or prognosis of disease, or the ability to predict or assess response to therapy. New methods for diagnosis and prognosis that harness the advances in the biologic sciences are needed.

### SUMMARY OF THE INVENTION

One aspect of this invention provides a method for determining the status of an individual. In some embodiments, the invention provides methods to determining the status of an individual by identifying a rare cell population associated with a status. In some embodiments, the status is a health status. In some embodiments, the invention provides a method of predicting a change in a health status in an individual from a first state to a second state comprising: determining the presence of a first and second class of cells in a sample from the individual, the presence being determined by a method comprising determining an activation level of an intracellular activatable element in single cells from said sample, classifying single cells into the first and second class, wherein at least one class is classified based on the activation level; calculating a ratio of the first and second class of cells and using the ratio to predict said change in health status; and predicting a change in a health status in the individual from a first state to a second state when said ratio exceeds a threshold number. In some embodiments, the threshold number expressed as a percentage is 30%. In some embodiments, the threshold number expressed as a percentage is 5%. In some embodiments threshold number expressed as a percentage is 1%. In some embodiments, the threshold number expressed as cell frequency is 10⁻². In some embodiments, the threshold number expressed as cell frequency is 10⁻³. In some embodiments, the threshold number expressed as cell frequency is 10⁻⁴.

In some embodiments, the second state is the location of an individual on a continuum that comprises normal, pre-pathological, and pathological states. In some embodiments, the pathological state of the continuum is an immunologic, malignant, or proliferative disorder or a combination thereof. In some embodiments, the status is a predicted response to a treatment for a pre-pathological or pathological condition, or a response to treatment for a pre-pathological or pathological condition.

In some embodiments, the pathological state is a malignant disorder. In some embodiments, the malignant disorder is a solid tumor or a hematologic malignancy. In some embodiments, the malignant disorder includes metastases. In some embodiments, the malignant disorder is non-B cell lineage derived. In some embodiments, the non-B cell lineage derived malignant disorder is selected from the group consisting of Acute Myeloid Leukemia (AML), Chronic Myeloid Leukemia (CML), non-B cell Acute Lymphocytic Leukemia (ALL), non-B cell lymphomas, myelodysplastic disorders, myeloproliferative disorders, myelofibroses, polycythemias, thrombocythemias, and non-B atypical immune lymphoproliferations. In some embodiments, the non-B cell lineage derived malignant disorder is AML.

In some embodiments, the pathological state is a malignant disorder that is derived from a B cell or B cell lineage. In some embodiments, the malignant disorder is a B-Cell or B cell lineage derived disorder is selected from the group consisting of Chronic Lymphocytic Leukemia (CLL), B cell lymphocyte lineage leukemia, B cell lymphocyte lineage lymphoma, Multiple Myeloma, and plasma cell disorders. In some embodiments, the B-Cell or B cell lineage derived disorder is CLL.

In some embodiments, the methods of the invention further comprise predicting a response to a treatment for a pre-pathological or pathological condition, or a response to treatment for a pre-pathological or pathological condition.

In some embodiments, the activation levels of a plurality of intracellular activatable elements in single cells are determined. In some embodiments, the activation level of at least about 2, 3, 4, 5, 6, 7, 8, 9, 10, or more than 10 intracellular (counting by whole numbers) activatable elements is determined.

In some embodiments, the plurality of cells obtained from the individual is first exposed to a modulator before determining said activation levels of said activatable element(s). In some embodiments, the plurality of cells is divided into separate groups and each group is subjected to a different modulator.

In some embodiments, the sample from the individual is a blood sample. In some embodiments, the sample is a biopsy sample or a surgical sample.

In some embodiments, calculating a ratio of the classes of cells comprises a determination of the number of cells in one or more particular classes of cells. In some embodiments, the status of the individual is determined by a process comprising determining whether or not the number of cells in one or more of said classes is greater than, less than, or equal to a threshold number. In some embodiments, the threshold number of cells in one or more classes is about 0, 1, 5, 10, 50, 100, 500, 1000, 10,000, 100,000, or 1,000,000. In some embodiments, determining the status of an individual comprises determining whether or not the number of cells in a class is greater than a threshold number of 0. In some embodiments, the class is a predefined class.

In some embodiments, the class is a class of cells wherein one or more activation levels of the cells are different when compared to determinations made in healthy control samples, or when compared to previous determinations made in a series of samples from said individual. In some embodiments, the one or more different activation levels comprise one or more additional activation levels compared to healthy controls or previous samples from said individual. In some embodiments, one or more different activation levels comprises one or fewer activation levels compared to healthy controls or previous samples from said individual.

In some embodiments, the ratio is determined by comparing the number of cells in one or more particular class or classes of cells to the number of cells in one or more other class or classes of cells, or to the total number of cells in the sample or a fraction of the sample. In some embodiments, the status is determined by a process comprising determining whether or not said ratio is greater than, less than, or equal to a threshold number. In some embodiments, the threshold ratio, expressed as a percentage, is about 0%, 0.0000001%, 0.000001%, 0.00001%, 0.0001%, 0.001%, 0.005%, 0.01%, 0.05%, 0.1%, 0.5%, 1.0%, 5.0%, 10%, 20%, or 30%.

In some embodiments, the determination of a status in an individual is performed on a plurality of samples from the individual. In some embodiments, the plurality of samples comprises samples from different locations in the individual, samples taken at different times from the individual, samples treated in different ways prior to determining the activation level, or a combination thereof. In some embodiments, the plurality of samples comprises a series of samples taken from the individual at different times.

In some embodiments, the method further comprises determining of the rate of change in the number of cells in one or more of said classes, or determining the rate of change of the ratio of the number of cells in one or more particular class or classes of cells to the number of cells in one or more other class or classes of cells, or to the total number of cells in the sample or a fraction of the sample. In some embodiments, the rate of change is expressed as the doubling time of said cells. In some embodiments, the status is determined by a process comprising analyzing said rate of change.

In some embodiments, the method of determining the status of an individual further comprises determining an appropriate course of treatment for said individual based on said status of the individual. In some embodiments, the appropriate course of treatment comprises watchful waiting, supportive therapy, initiating a therapy, not initiating a therapy, stopping, shortening, prolonging, or modifying an existing therapy, adding an additional therapy to existing therapy, or combinations of the foregoing. In some embodiments, therapy is selected from the group consisting of surgical excision, transplantation, or the administration of a physical, chemical, or biological agent, or combinations thereof.

In some embodiments, one or more characteristics of the individual is determined, and the status of the individual is then determined based on both quantitative analysis of classes of cells and the one or more characteristics of the individual. In some embodiments, the determination of an appropriate course of treatment is also based on one or more characteristics of the individual. In some embodiments, the one or more characteristics comprise physical characteristics, clinical status, treatment characteristics, and biochemical/molecular markers.

In some embodiments, the modulator is an activator or an inhibitor. In some embodiments, the modulator is a growth factor, cytokine, adhesion molecule modulator, hormone, small molecule, polynucleotide, antibody, natural compound, lactone, chemotherapeutic agent, immune modulator, carbohydrate, protease, ion, reactive oxygen species, or radiation. In some embodiments, the modulator is a B cell receptor modulator. In some embodiments, the B cell receptor modulator is a B cell receptor activator. In some embodiments, the B cell receptor activator is a cross-linker of the B cell receptor complex or the B cell co-receptor complex.

In some embodiments, the cross-linker is an antibody or a molecular binding entity. In some embodiments, the modulator is an inhibitor that inhibits a cellular factor or a plurality of factors that participates in a signaling cascade in the cell. In some embodiments, the inhibitor is a phosphatase inhibitor. In some embodiments, the phosphatase inhibitor is H₂O₂.

In some embodiments, the cells are further subjected to a second modulator. In some embodiments, the two modulators are a B cell receptor activator and a phosphatase inhibitor. In some embodiments, the modulators are F(ab)₂IgM or biotinylated F(ab)₂IgM and H₂O₂.

In some embodiments, the activation state is selected from the group consisting of cleavage by extracellular or intracellular protease exposure, novel hetero-oligomer formation, glycosylation state, phosphorylation state, acetylation state, methylation state, biotinylation state, glutamylation state, glycylation state, hydroxylation state, isomerization state, prenylation state, myristoylation state, lipoylation state, phosphopantetheinylation state, sulfation state, ISGylation state, nitrosylation state, palmitoylation state, SUMOylation state, ubiquitination state, neddylation state, citrullination state, deamidation state, disulfide bond formation state, proteolytic cleavage state, translocation state, changes in protein turnover, multi-protein complex state, oxidation state, multi-lipid complex, and biochemical changes in cell membrane. In some embodiments, the activation state is a phosphorylation state. In some embodiments, the activatable element is selected from the group consisting of proteins, carbohydrates, lipids, nucleic acids and metabolites. In some embodiments, the activatable element is a protein. In some embodiments, the protein is a protein subject to phosphorylation and/or dephosphorylation. In some embodiments, the protein is selected from the group consisting of kinases, phosphatases, lipid signaling molecules, adaptor/scaffold proteins, cytokines, cytokine regulators, ubiquitination enzymes, adhesion molecules, cytoskeletal proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, pS6, 4EPB-1, eIF4E-binding protein, RNA polymerase, initiation factors, and elongation factors. In some embodiments, the protein is selected from the group consisting of Erk, Syk, Zap70, Lyn, Btk, BLNK, Cbl, PLCγ2, Akt, RelA, p38, S6. In some embodiments, the protein is S6. In some embodiments, the activatable element is responsive to a change in metabolic state, temperature, local ion concentration, or heterologous protein expression.

In some embodiments, the activation level is determined by a process comprising the binding of a binding element which is specific to a particular activation state of the particular activatable element. In some embodiments, the binding element comprises a protein. In some embodiments, the protein is an antibody. In some embodiments, the antibody binds to an activatable element selected from the group consisting of kinases, phosphatases, adaptor/scaffold proteins, ubiquitination enzymes, adhesion molecules, contractile proteins, cytoskeletal proteins, heterotrimeric G proteins, small molecular weight GTPases, guanine nucleotide exchange factors, GTPase activating proteins, caspases and proteins involved in apoptosis, ion channels, molecular transporters, molecular chaperones, metabolic enzymes, vesicular transport proteins, hydroxylases, isomerases, transferases, deacetylases, methylases, demethylases, proteases, esterases, hydrolases, DNA binding proteins and transcription factors.

In some embodiments, the antibody binds to an activatable element selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phospholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, pS6, 4EPB-1, eIF4E-binding protein, RNA polymerase, initiation factors, and elongation factors.

In some embodiments, the step of finding the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation levels of the plurality of intracellular activatable elements in single cells. In some embodiments, the determining step comprises the use of flow cytometry. In some embodiments, the classifying of single cells is further based on the presence or absence of one or more cell surface markers, intracellular markers, or combinations thereof.

In another aspect, the invention provides a method of detecting the presence or absence of disease-associated cells in an individual who has received treatment comprising: subjecting a plurality of cells in a sample from said individual to a modulator; determining the response of single cells in the plurality of cells to said modulator; and determining the presence or absence of the disease-associated cells based on the response. In some embodiments, the method further comprises determining the status of the individual based on said presence or absence of disease-associated cells. In some embodiments, the disease associated cells are rare cells.

In some embodiments, the response to the modulator comprises determining the activation level of an intracellular activatable element in said single cells. In some embodiments, the method further comprises dividing the sample into a plurality of subsamples, and subjecting each subsample to a different modulator.

In some embodiments, the invention provides a method of detecting the minimal residual status of a disease in an individual who has received treatment comprising subjecting a plurality of cells in a sample from an individual to a modulator; determining the activation levels of a plurality of intracellular activatable elements in single cells in response to the modulator by a process comprising the binding of a plurality of binding elements which are specific to a particular activation state of a particular activatable element, wherein the single cells are placed into one or more classes based on said response to said modulator or modulators; determining the presence or absence of said disease-associated cells based on the response, wherein determining the presence or absence of the disease-associated cells comprises quantitative analysis of the one or more classes; and determining the minimal residual status of a disease, wherein the minimal residual status is based on the presence or absence of a small number of the disease-associated cells. The minimal residual status refers to the number of disease-associated cells that remain in the individual during treatment or after treatment when the individual is in remission. In some embodiments, the minimal residual status of a disease in an individual is used to determine a health status in the individual.

In some embodiments, determining the response to the modulator comprises determining the activation levels of a plurality of intracellular activatable elements in said single cells. In some embodiments, the activation level of at least 2, 3, 4, 5, 6, 7, 8, 9, 10, or more than 10 (counting by whole numbers) intracellular activatable elements is determined. In some embodiments, the single cells are placed into one or more classes based on said response to said modulator or modulators. In some embodiments, the classes are predefined classes.

In some embodiments, the determining of the presence or absence of said disease-associated cells comprises quantitative analysis of classes. In some embodiments, the classes are predefined classes. In some embodiments, the quantitative analysis of classes comprises determining whether or not said number of said cells in one or more of said classes is greater than, less than, or equal to a threshold number. In some embodiments, the threshold number is about 0, 1, 5, 10, 50, 100, 500, 1000, 10,000, 100,000, or 1,000,000. In some embodiments, the method comprises determining whether or not said number of cells in a class is greater than the threshold number 0.

In some embodiments, the method further comprises the determination of the ratio of the number of cells in one or more particular class or classes of cells to the number of cells in one or more other class or classes of cells, or to the total number of cells in the sample or a fraction of the sample. In some embodiments, detecting the presence or absence of disease-associated cells is determined by a process comprising determining whether or not said ratio is greater than, less than, or equal to a threshold number. In some embodiments, the threshold ratio, expressed as a percentage, is about 0%, 0.0000001%, 0.000001%, 0.00001%, 0.0001%, 001%, 0.005%, 0.01%, 0.05%, 0.1%, 0.5%, 1.0%, 5.0%, 10%, 20%, 40%, 60%, 80%, 90%, 95%, or 100%.

In some embodiments, the quantitative analysis is performed on a plurality of samples from said individual. In some embodiments, the plurality of samples comprises samples from different locations in the individual, samples taken at different times from the individual, samples treated in different ways prior to determining the activation level, or a combination thereof. In some embodiments, the plurality of samples comprises a series of samples taken from the individual at different times.

In some embodiments, the method further comprises determining the rate of change in the number of cells in one or more of said classes, or determining the rate of change of the ratio of the number of cells in one or more particular class or classes of cells to the number of cells in one or more other class or classes of cells, or to the total number of cells in the sample or a fraction of the sample.

In some embodiments, the method further comprises determining an appropriate course of treatment for said individual based on said status of the individual. In some embodiments, the appropriate course of treatment comprises watchful waiting, supportive therapy, initiating a therapy, not initiating a therapy, stopping, shortening, prolonging, or modifying an existing therapy, adding an additional therapy to existing therapy, or combinations of the foregoing.

In some embodiments, the individual has received treatment for a malignant disorder. In some embodiments, the malignant disorder is a solid tumor or a hematologic malignancy. In some embodiments, the malignant disorder is non-B cell lineage derived. In some embodiments, the non-B cell lineage derived malignant disorder is selected from the group consisting of Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B cell atypical immune lymphoproliferations. In some embodiments, the non-B cell lineage derived malignant disorder is AML.

In some embodiments, the malignant disorder is a B cell or B cell lineage derived disorder. In some embodiments, the malignant disorder is a B-Cell or B cell lineage derived disorder is selected from the group consisting of Chronic Lymphocytic Leukemia (CLL), B cell lymphocyte lineage leukemia, B cell lymphocyte lineage lymphoma, Multiple Myeloma, and plasma cell disorders. In some embodiments, the B-Cell or B cell lineage derived disorder is CLL.

In some embodiments, the status is expressed as a likelihood of return or progression of a condition, or likelihood of a new condition developing.

In some embodiments, the modulator is an activator or an inhibitor. In some embodiments, the modulator is a growth factor, cytokine, adhesion molecule modulator, hormone, small molecule, polynucleotide, antibody, natural compound, lactone, chemotherapeutic agent, immune modulator, carbohydrate, protease, ion, reactive oxygen species, or radiation. In some embodiments, the modulator is a B cell receptor modulator. In some embodiments, the B cell receptor modulator is a B cell receptor activator. In some embodiments, the B cell receptor activator is a crosslinker is selected from the group consisting of F(ab)₂ IgM, IgG, IgD, polyclonal BCR antibodies, monoclonal BCR antibodies, Fc receptor derived binding elements.

In some embodiments, the modulator is an inhibitor, and wherein said inhibitor is an inhibitor of a cellular factor or a plurality of factors that participates in a signaling cascade in said cell. In some embodiments, the inhibitor is a phosphatase inhibitor. In some embodiments, the phosphatase inhibitor is selected from the group consisting of H₂O₂, siRNA, miRNA, Cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Perm olybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenyarsine oxide, Pyrrolidine Dithiocarbamate, and Aluminium fluoride. In some embodiments, the phosphatase inhibitor is H₂O₂.

In some embodiments, the method further comprises subjecting the cells to a second modulator concurrently with the first modulator. In some embodiments, the modulators are a B cell receptor activator and a phosphatase inhibitor. In some embodiments, the modulators are F(ab)₂IgM or biotinylated F(ab)₂IgM and H₂O₂.

In some embodiments, the activation level is based on the activation state selected from the group consisting of cleavage by extracellular or intracellular protease exposure, novel hetero-oligomer formation, glycosylation state, phosphorylation state, acetylation state, methylation state, biotinylation state, glutamylation state, glycylation state, hydroxylation state, isomerization state, prenylation state, myristoylation state, lipoylation state, phosphopantetheinylation state, sulfation state, ISGylation state, nitrosylation state, palmitoylation state, SUMOylation state, ubiquitination state, neddylation state, citrullination state, deamidation state, disulfide bond formation state, proteolytic cleavage state, translocation state, changes in protein turnover, multi-protein complex state, oxidation state, multi-lipid complex, and biochemical changes in cell membrane. In some embodiments, the activation state is a phosphorylation state.

In some embodiments, the activatable element is selected from the group consisting of proteins, carbohydrates, lipids, nucleic acids and metabolites. In some embodiments, the activatable element is a protein. In some embodiments, the protein is a protein subject to phosphorylation and/or dephosphorylation.

In some embodiments, the protein is selected from the group consisting of kinases, phosphatases, lipid signaling molecules, adaptor/scaffold proteins, cytokines, cytokine regulators, ubiquitination enzymes, adhesion molecules, cytoskeletal proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, pS6, 4EPB-1, eIF4E-binding protein, RNA polymerase, initiation factors, and elongation factors. In some embodiments, the protein is selected from the group consisting of Erk, Syk, Zap70, Lyn, Btk, BLNK, Cbl, PLCγ2, Akt, RelA, p38, S6. In some embodiments, the protein is S6.

In some embodiments, the activation level is determined by a process comprising the binding of a binding element which is specific to a particular activation state of the particular activatable element. In some embodiments, the binding element comprises a protein. In some embodiments, the protein is an antibody. In some embodiments, the antibody binds to a activatable element selected from the group consisting of kinases, phosphatases, adaptor/scaffold proteins, ubiquitination enzymes, adhesion molecules, contractile proteins, cytoskeletal proteins, heterotrimeric G proteins, small molecular weight GTPases, guanine nucleotide exchange factors, GTPase activating proteins, caspases and proteins involved in apoptosis, ion channels, molecular transporters, molecular chaperones, metabolic enzymes, vesicular transport proteins, hydroxylases, isomerases, transferases, deacetylases, methylases, demethylases, proteases, esterases, hydrolases, DNA binding proteins and transcription factors.

In some embodiments, the antibody binds to an activatable element selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3a, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, pS6, 4EPB-1, eIF4E-binding protein, RNA polymerase, initiation factors, and elongation factors.

In some embodiments, the step of determining the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells. In some embodiments, the determining step comprises the use of flow cytometry.

In some embodiments, determining the presence or absence of the disease-associated cells is further based on the presence or absence of one or more cell surface markers, the presence or absence of one or more intracellular markers, or a combination thereof.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph illustrating the change in the number of a predefined class of cells over time. Here, the cell number is increasing and by the sixth measurement has exceeded the threshold number.

FIG. 2 illustrates the detection and quantification of multiple predefined classes of cells in a sample. 2A. Numerous predefined classes can be observed and quantified when multiple binding elements to intracellular activatable elements are employed, particularly if physical parameters like cell volume or density and additional biochemical information such as the expression level of cell surface markers or nuclear antigens is employed. 2B Various comparisons can be made between classes including taking the ratio of the cell numbers found in particular classes.

FIG. 3 is a graph illustrating the change in the ratio of predefined classes over time. Here, the ratio has decreased over time and by the fourth measurement has dropped below the threshold number

FIG. 4 is a graph illustrating the rate of change in the cell number two different predefined classes of cells over time. In one cell population, illustrated by the thick line, the rate of change in the cell population is decreasing, while in the other population, illustrated by the thin line, the rate of change is increasing.

FIG. 5 shows identification of relevant subpopulations in BMMCs from MDS patients. Myeloblasts, mature monocytes, nRBCs, and lymphocytes are gated based on CD45, CD235ab, CD71, CD34, CD33 and CD11b expression as well as FSC and SSC profiles.

FIG. 6 shows identification of erythroid cells at different developmental stages from normal and MDS patient bone marrow based on their CD235ab and CD71 expression profiles.

FIG. 7 shows analysis of erythroid precursors in normal versus MDS bone marrow. The results reveal a block of erythroid differentiation in MDS.

FIG. 8 shows STAT5 and STAT1 phosphorylation in rRBCs from normal and MDS patients in response to erythropoietin (EPO) stimulation. nRBC subpopulation from MDS patients exhibits STAT5 phosphorylation in response to EPO stimulation.

FIG. 9 shows STAT5 and STAT1 phosphorylation in rRBCs from normal and MDS patients in response to interferon gamma (IFNγ) stimulation. nRBC subpopulation from MDS patients exhibits STAT1 phosphorylation in response to IFNγ stimulation.

FIG. 10 shows a concentration dependent loss of CD34+ myeloblast cells in healthy BMMCs in the presence of 5-Azacytidine.

FIG. 11 shows that Decitabine (Dacogen) does not affect the viability of CD34+ myeloblast cells.

FIG. 12 shows a concentration dependent loss of CD34+ myeloblast cells in healthy BMMCs in the presence of Vorinostat (Zolinza).

FIG. 13 shows CD45RA/RO/RB expression profiles of mature monocytes, myeloblasts, and lymphocytes

FIG. 14 shows CD45RA/RO/RB expression profiles of mature monocytes, myeloblasts, and lymphocytes from bone marrow of MDS patient 03.

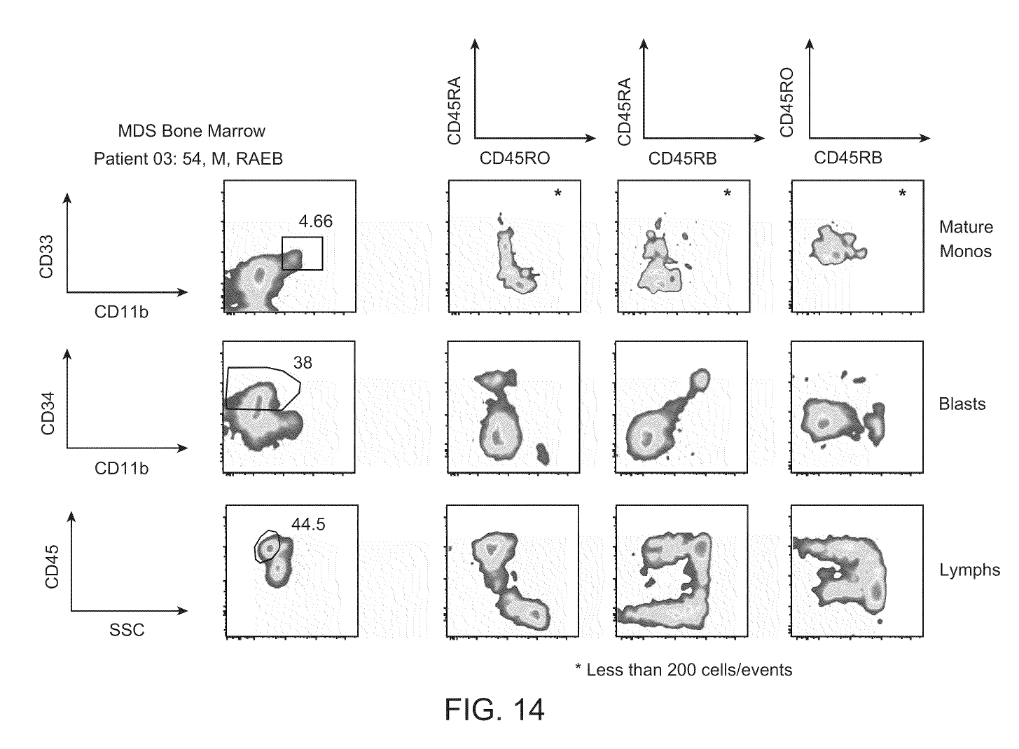

FIG. 15 is a diagram showing the method of determining a status of an individual at different stages. The method can be applied to an individual before a diagnosis, an individual undergoing a treatment, or an individual undergoing remission or having a relapse.

FIG. 16 shows p-Stat5 and p-Stat1 levels in myeloid cells from a patient at the time of diagnosis or at relapse.

FIG. 17 shows p-AKT and p-S6 levels in myeloid cells from a patient at the time of diagnosis and post induction therapy.

FIG. 18 shows p-AKT and p-S6 levels in CD33, CD11b⁻, CD34⁺ cells in an AML patient.

FIG. 19 shows the frequency of p-AKT/pS6 myeloid cells responsive to SCF in different AML patients.

### DETAILED DESCRIPTION OF THE INVENTION

The present invention incorporates information disclosed in other applications and texts. The following patent and other publications are hereby incorporated by reference in their entireties: Haskell et al, Cancer Treatment, 5^{th} Ed., W.B. Saunders and Co., 2001; Alberts et al., The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Immunobiology, Janeway et al. 7^{th} Ed., Garland, and Leroith and Bondy, Growth Factors and Cytokines in Health and Disease, A Multi Volume Treatise, Volumes 1A and 1B, Growth Factors, 1996. Patent applications that are also incorporated by reference include U.S. Ser. Nos. 10/193,462; 11/655,785; 11/655,789; 10/346,620; 11/655,821; 10/898,734; and 11/338,957. Some commercial reagents, protocols, software and instruments that are useful in some embodiments of the present invention are available at the Becton Dickinson Website http://www.bdbiosciences.com/features/products/, and the Beckman Coulter website, http://www.beckmancoulter.com/Default.asp?bhfv=7. Relevant articles include High-content single-cell drug screening with phosphospecific flow cytometry, Krutzik et al., Nature Chemical Biology, 23 Dec. 2007; Irish et al., Flt3 Y591 duplication and Bcl-2 over expression are detected in acute myeloid leukemia cells with high levels of phosphorylated wild-type p53, Neoplasia, 2007, and Irish et al., Single cell profiling of potentiated phospho-protein networks in cancer cells, Cell, Vol. 118, 1-20 Jul. 23, 2004; Schulz, K. R., et al., Single-cell phospho-protein analysis by flow cytometry, Curr Protoc Immunol, 2007, 78:8 8.17.1-20; Krutzik, P. O., et al., Coordinate analysis of murine immune cell surface markers and intracellular phosphoproteins by flow cytometry, J. Immunol. 2005 Aug. 15; 175(4):2357-65; Krutzik, P. O., et al., Characterization of the murine immunological signaling network with phosphospecific flow cytometry, J. Immunol. 2005 Aug. 15; 175(4):2366-73; and Krutzik, P. O. and Nolan, G. P., Intracellular phospho-protein staining techniques for flow cytometry: monitoring single cell signaling events, Cytometry A. 2003 October; 55(2):61-70. Experimental and process protocols and other helpful information can be found at http:/proteomices.stanford.edu.

One embodiment of the invention is directed to methods for determining the status of an individual by determining the activation level of individual cells in one or more samples obtained from the individual. Typically, the status of an individual will be the health status, but any type of status can be determined if it can be correlated to the status of single cells in a sample from the individual. In some embodiments, the invention provides methods for determining the status of an individual by detecting one or more rare cell populations. Thus, the invention provides methods for the determination of the status of an individual by analyzing one or more rare populations of cells, usually not detectable by other methods known in the art, while keeping a high level of statistical significance in the determination. In some embodiments, the invention provides methods for early determination of the individual status. For example, in the case of diagnosis of a pathological state the invention provides for early diagnosis of the pathological state, e.g., before the individual presents any symptoms.

In some embodiments the status of the individual is the minimal status of a pathological state. Thus, in some embodiments, the invention is directed to determining the minimal status of a pathological state in an individual by determining the activation level of individual cells in one or more samples obtained from the individual. The “minimal status” of a pathological state as used herein refers to the minimum number of cells indicative of a pathological state. In some embodiments, the minimal status of a pathological state in the minimum numbers of cells required to make a diagnosis for the pathological state. In certain instances, the finding of 0 cells associated with a pathological state may be determinative as to minimal status of a pathological state. For example, the finding of 0 cells associated with a pathological state provides evidence that the individual does not have the pathological state or has not experienced a recurrence. In some embodiments, the presence of 1 cell associated with a pathological state may be determinative of an individual's status. In this case, the threshold number is 0, and finding even a single cell (more than zero) is indicative of the minimal status of the pathological state. For example, the finding of 1 cell that is associated with a highly malignant cancer phenotype indicates that the in the case of cancer, the disease process has begun, but may be yet to manifest disease symptoms. In an individual who has been treated for the pathological condition, the detection of cells associated with the pathological state indicates that treatment is incomplete. In other instances, a finding of a number higher than a threshold of cells associated with a pathological state may be determinative of an individual's status, wherein the threshold in the minimum number of cells required to make a determination of the individual's status. For example, a finding of equal or higher that 10⁻⁴ cells associated with a cancer phenotype may indicate that the individual is at risk of having a relapse, whereas a finding of less than 10⁻⁴ cells may indicate that the individual is at very low risk of relapse.

In some embodiments, the status of the single cells in the sample is determined, e.g., by determining the status of one or more activatable elements in the cells. The activatable elements may be proteins; in some embodiments, the activatable elements are phosphoproteins. The cells may then be classified into one or more classes, depending on the activation level of the one or more activatable elements, and a quantitative analysis is performed on the number of cells in one or more of the classes. In some embodiments, cells are treated with a modulator before their status is determined. See U.S. Ser. No. 10/898,734.

In some embodiments, the health status of an individual places the individual along a health continuum that typically runs from a healthy state to one or more pre-pathologic states, and finally to a pathologic state. In some instances, the health continuum may run from a healthy state to a pathological state without an intervening pre-pathologic state. The health continuum may also comprise a partial continuum of the aforementioned states or a portion of one state. The health continuum may be related to the general health status of an individual, an organ or organ system or the individual component tissues of an organ. Additionally, the health continuum may be specific for a family of related diseases or disorder, a particular disease or disorder or a subtype of a disease or disorder. See Haskell et al, Cancer Treatment, 5^{th} Ed., W.B. Saunders and Co., 2001

Diseases, disorders, and conditions encompassed by a health continuum can include an immunologic, malignant, or proliferative disease or disorder, or one that has characteristics from a combination of these disorders. See Immunobiology, Janeway et al. 7^{th} Ed., Garland. Diseases that are especially likely to progress along a continuum from health to prepathological to pathological are cancers, which typically require a series of genetic changes in order to progress to malignancy. Cancers that are especially amenable to evaluation and intervention include those that are associated with the blood, i.e., hematologic malignancies, because blood is easily sampled and processed. An example of a malignancy that progresses along such a continuum, which serves as an example of disorders that may be evaluated by the methods of the invention, is AML. AML can be preceded by a prepathological stage, myelodysplastic disorder (MDS). The methods of the invention allow monitoring of an individual at a series of time points to determine where on the continuum from healthy, through MDS (prepathological) to AML (pathological), the individual is situated. See Haskell et al, Cancer Treatment, 5^{th} Ed., W.B. Saunders and Co., 2001

Knowing the health status of an individual allows for the diagnosis, prognosis, choice or modification of treatment, and/or monitoring of a disease, disorder, or condition. Through the determination of the health status of an individual, a health care practitioner can assess whether the individual is in the normal range for a particular condition or whether the individual has a pre-pathological or pathological condition warranting monitoring and/or treatment. This type of methodology can be particularly important with diseases or conditions where an individual is asymptomatic and appears normal. This is often the case with many types of cancer, which may be asymptomatic for months or years and which, at the time symptoms appear, may be much less amenable to treatment than if they had been detected earlier.

The determination of the health status may also indicate response of an individual to treatment for a condition. Such information allows for ongoing monitoring of the condition and/or additional treatment. In one embodiment, the invention provides for the detection of the presence of disease-associated cells or the absence or reduction of cells necessary for normal physiology in an individual that is being treated, or was previously treated, for the disease or condition. The disease-associated cells may be cancerous and may be present at sufficiently low numbers so as not to cause overt symptoms or be detectable by imaging modalities, clinical exam, or routine clinical screening labs e.g. complete blood count. In some embodiments, the invention provides for the detection of a slight reduction in a normal cell population that precedes or accompanies a disease process. In some embodiments the disease process comprises a malignancy.

In some embodiments, the determination of the health status of an individual may be used to ascertain whether a previous condition or treatment has induced a new pre-pathological or pathological condition that requires monitoring and/or treatment. For example, treatment for many forms of cancers (e.g. lymphomas and childhood leukemias) can induce certain adult leukemias, and the methods of the present invention allow for the early detection and treatment of such leukemias.

In another embodiment, the status of an individual can indicate an individual's predicted or actual response to treatment for a pre-pathological or pathological condition. This predictive information can be obtained through the analysis of the same, additional or different parameters than those used to place the individual along the health continuum. Predictive information may be used to determine the best therapy for an individual, which may include the determination that the best therapy for a patient is supportive care.

In a further embodiment, the status of an individual may indicate an individual's immunologic status and may reflect a general immunologic status, an organ or tissue specific status, or a disease related status.

### Samples and Sampling

The methods involve analysis of one or more samples from an individual. An individual is any multicellular organism; in some embodiments, the individual is an animal, e.g., a mammal. In some embodiments, the individual is a human.

The sample may be any suitable type that allows for the analysis of single cells. Samples may be obtained once or multiple times from an individual. Multiple samples may be obtained from different locations in the individual (e.g., blood samples, bone marrow samples and/or lymph node samples), at different times from the individual (e.g., a series of samples taken to monitor response to treatment or to monitor for return of a pathological condition), or any combination thereof. These and other possible sampling combinations based on the sample type, location and time of sampling allows for the detection of the presence of pre-pathological or pathological cells, the measurement treatment response and also the monitoring for disease.

When samples are obtained as a series, e.g., a series of blood samples obtained after treatment, the samples may be obtained at fixed intervals, at intervals determined by the status of the most recent sample or samples or by other characteristics of the individual, or some combination thereof. For example, samples may be obtained at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for sampling and the availability of sampling facilities, thus approximate intervals corresponding to an intended interval scheme are encompassed by the invention. As an example, an individual who has undergone treatment for a cancer may be sampled (e.g., by blood draw) relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, or during the sampling, sampling intervals may be modified.

Generally, the most easily obtained samples are fluid samples. Fluid samples include normal and pathologic bodily fluids and aspirates of those fluids. Fluid samples also comprise rinses of organs and cavities (lavage and perfusions). Bodily fluids include whole blood, bone marrow aspirate, synovial fluid, cerebrospinal fluid, saliva, sweat, tears, semen, sputum, mucus, menstrual blood, breast milk, urine, lymphatic fluid, amniotic fluid, placental fluid and effusions such as cardiac effusion, joint effusion, pleural effusion, and peritoneal cavity effusion (ascites). Rinses can be obtained from numerous organs, body cavities, passage ways, ducts and glands. Sites that can be rinsed include lungs (bronchial lavage), stomach (gastric lavage), gastrointestinal track (gastrointestinal lavage), colon (colonic savage), vagina, bladder (bladder irrigation), breast duct (ductal savage), oral, nasal, sinus cavities, and peritoneal cavity (peritoneal cavity perfusion). In some embodiments the sample or samples is blood.

Solid tissue samples may also be used, either alone or in conjunction with fluid samples. Solid samples may be derived from individuals by any method known in the art including surgical specimens, biopsies, and tissue scrapings, including cheek scrapings. Surgical specimens include samples obtained during exploratory, cosmetic, reconstructive, or therapeutic surgery. Biopsy specimens can be obtained through numerous methods including bite, brush, cone, core, cytological, aspiration, endoscopic, excisional, exploratory, fine needle aspiration, incisional, percutaneous, punch, stereotactic, and surface biopsy.

In some embodiments, the sample is a blood sample. In some embodiments, the sample is a bone marrow sample. In some embodiments, the sample is a lymph node sample. In some embodiments, the sample is cerebrospinal fluid. In some embodiments, combinations of one or more of a blood, bone marrow, cerebrospinal fluid, and lymph node sample are used.

In one embodiment, a sample may be obtained from an apparently healthy individual during a routine checkup and analyzed so as to provide an assessment of the individual's general health status. In another embodiment, a sample may be taken to screen for commonly occurring diseases. Such screening may encompass testing for a single disease, a family of related diseases or a general screening for multiple, unrelated diseases. Screening can be performed weekly, bi-weekly, monthly, bimonthly, every several months, annually, or in several year intervals and may replace or complement existing screening modalities.

In another embodiment, an individual with a known increased probability of disease occurrence may be monitored regularly to detect for the appearance of a particular disease or class of diseases. An increased probability of disease occurrence can be based on familial association, age, previous genetic testing results, or occupational, environmental or therapeutic exposure to disease causing agents. Breast and ovarian cancer related to inherited mutations in the genes BRCA1 and BRCA2 are examples of diseases with a familial association wherein susceptible individuals can be identified through genetic testing. Another example is the presence of inherited mutations in the adenomatous polyposis coli gene predisposing individuals to colorectal cancer. Examples of environmental or therapeutic exposure include individuals occupationally exposed to benzene that have increased risk for the development of various forms of leukemia, and individuals therapeutically exposed to alkylating agents for the treatment of earlier malignancies. Individuals with increased risk for specific diseases can be monitored regularly for the first signs of an appearance of an abnormal cell population. Monitoring can be performed weekly, bi-weekly, monthly, bimonthly, every several months, annually, or in several year intervals, or any combination thereof. Monitoring may replace or complement existing screening modalities. Through routine monitoring, early detection of the presence of disease causative or associated cells may result in increased treatment options including treatments with lower toxicity and increased chance of disease control or cure.

In a further embodiment, testing can be performed to confirm or refute the presence of a suspected genetic or physiologic abnormality associated with increased risk of disease. Such testing methodologies can replace other confirmatory techniques like cytogenetic analysis or fluorescent in situ histochemistry (FISH). In still another embodiment, testing can be performed to confirm or refute a diagnosis of a pre-pathological or pathological condition.

In instances where an individual has a known pre-pathologic or pathologic condition, a plurality of single cells from the appropriate location can be sample and analyzed to predict the response of the individual to available treatment options. In one embodiment, an individual treated with the intent to reduce in number or ablate cells that are causative or associated with a pre-pathological or pathological condition can be monitored to assess the decrease in such cells over time. A reduction in causative or associated cells may or may not be associated with the disappearance or lessening of disease symptoms. If the anticipated decrease in cell number does not occur, further treatment with the same or a different treatment regiment may be warranted.

In another embodiment, an individual treated to reverse or arrest the progression of a pre-pathological condition can be monitored to assess the reversion rate or percentage of cells arrested at the pre-pathological status point. If the anticipated reversion rate is not seen or cells do not arrest at the desired pre-pathological status point further treatment with the same or a different treatment regiment can be considered.

In a further embodiment, cells of an individual can be analyzed to see if treatment with a differentiating agent has pushed a cell type along a specific tissue lineage and to terminally differentiate with subsequent loss of proliferative or renewal capacity. Such treatment may be used preventively to keep the number of dedifferentiated cells associated with disease at a low level thereby preventing the development of overt disease. Alternatively, such treatment may be used in regenerative medicine to coax or direct pluripotent or multipotent stem cells down a desired tissue or organ specific lineage and thereby accelerate or improve the healing process.

Individuals may also be monitored for the appearance or increase in cell number of another predefined class or classes of cells that are associated with a good prognosis. If a beneficial, predefined class of cells is observed, measures can be taken to further increase their numbers, such as the administration of growth factors. Alternatively, individuals may be monitored for the appearance or increase in cell number of another predefined class or classes of cells associated with a poor prognosis. In such a situation, renewed therapy can be considered including continuing, modifying the present therapy or initiating another type of therapy.

In these embodiments, one or more samples may be taken from the individual, and subjected to a modulator, as described herein. In some embodiments, the sample is divided into subsamples that are each subjected to a different modulator. After treatment with the modulator, single cells in the sample or subsample are analyzed to determine their activation level(s). Any suitable form of analysis that allows a determination of cell activation level(s) may be used. In some embodiments, the analysis includes the determination of the activation level of an intracellular element, e.g., a protein. In some embodiments, the analysis includes the determination of the activation level of an activatable element, e.g., an intracellular activatable element such as a protein, e.g., a phosphoprotein. Determination of the status may be achieved by the use of activation state-specific binding elements, such as antibodies, as described herein. A plurality of activatable elements may be examined. Single cells may be placed into predefined classes, and the status of the individual determined based on the classes into which cells are categorized. In some embodiments, a quantitative analysis of the number of cells in one or more classes is performed to determine the status of the individual.

Certain fluid samples can be analyzed in their native state with or without the addition of a diluent or buffer. Alternatively, fluid samples may be further processed to obtain enriched or purified cell populations prior to analysis. Numerous enrichment and purification methodologies for bodily fluids are known in the art. A common method to separate cells from plasma in whole blood is through centrifugation using heparinized tubes. By incorporating a density gradient, further separation of the lymphocytes from the red blood cells can be achieved. A variety of density gradient media are known in the art including sucrose, dextran, bovine serum albumin (BSA), FICOLL diatrizoate (Pharmacia), FICOLL metrizoate (Nycomed), PERCOLL (Pharmacia), metrizamide, and heavy salts such as cesium chloride. Alternatively, red blood cells can be removed through lysis with an agent such as ammonium chloride prior to centrifugation.

Whole blood can also be applied to filters that are engineered to contain pore sizes that select for the desired cell type or class. For example, rare pathogenic cells can be filtered out of diluted, whole blood following the lysis of red blood cells by using filters with pore sizes between 5 to 10 μm, as disclosed in U.S. patent application Ser. No. 09/790,673. Alternatively, whole blood can be separated into its constituent cells based on size, shape, deformability or surface receptors or surface antigens by the use of a microfluidic device as disclosed in U.S. patent application Ser. No. 10/529,453.

Select cell populations can also be enriched for or isolated from whole blood through positive or negative selection based on the binding of antibodies or other entities that recognize cell surface or cytoplasmic constituents. For example, U.S. Pat. No. 6,190,870 to Schmitz et al. discloses the enrichment of tumor cells from peripheral blood by magnetic sorting of tumor cells that are magnetically labeled with antibodies directed to tissue specific antigens.

Solid tissue samples may require the disruption of the extracellular matrix or tissue stroma and the release of single cells for analysis. Various techniques are known in the art including enzymatic and mechanical degradation employed separately or in combination. An example of enzymatic dissociation using collagenase and protease can be found in Wolters G H J et al. An analysis of the role of collagenase and protease in the enzymatic dissociation of the rat pancrease for islet isolation. Diabetologia 35:735-742, 1992. Examples of mechanical dissociation can be found in Singh, N P. Technical Note: A rapid method for the preparation of single-cell suspensions from solid tissues. Cytometry 31:229-232 (1998). Alternately, single cells may be removed from solid tissue through microdissection including laser capture microdissection as disclosed in Laser Capture Microdissection, Emmert-Buck, M. R. et al. Science, 274(8):998-1001, 1996.

In some embodiments, single cells can be analyzed within a tissue sample, such as a tissue section or slice, without requiring the release of individual cells before determining step is performed.

### Modulators

In some embodiments the sample may be treated with at least one modulator. Such treatment can yield information regarding the state of single cells that is useful in determining the status of the individual. In some embodiments, the sample is divided into subsamples which are each treated with a different modulator. A modulator causes modification of one or more activatable elements of a cell (e.g., activation or deactivation), a change in expression of an element, or the localization of an element, generally as part of a signaling pathway, in at least one type of cell. A modulator may be an activator or an inhibitor—e.g., a modulator may activate one or more activatable elements in one or more cellular signaling pathways, or inhibit one or more activatable elements in one or more cellular pathways. See U.S. Ser. Nos. 10/193,462; 11/655,785; 11/655,789; 10/346,620; 11/655,821; 10/898,734; and 11/338,957.

Cells can be treated with a modulator as a single pulse, or with sequential pulses. With sequential treatment, a modulator can be used at the same concentration and duration of exposure or at different concentrations and exposure. In some embodiments, cells are treated with two modulators. In some embodiments, cells are treated with 3, 4, 5, 6, 7, 8, 9, 10, or more modulators. These modulators can both be activators, inhibitors, or one can be an activator and the other an inhibitor. Treatment can consist of simultaneous or sequential exposure to a combination of modulators. As an illustrative example, a cell can be treated simultaneously with a B cell receptor activator such as F(ab)₂IgM and a phosphatase inhibitor like H₂O₂.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which is added a modulator. In some embodiments, cells are modulated after processing for single cells or purified fractions of single cells. As an illustrative example, whole blood can be collected and processed for an enriched fraction of lymphocytes that is then exposed to a modulator.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, neurotransmitters, adhesion molecules, hormones, small molecules, inorganic compounds, polynucleotides, antibodies, natural compounds, lectins, lactones, chemotherapeutic agents, biological response modifiers, carbohydrate, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

Modulators that are activators include ligands for cell surface receptors such as hormones, growth factors and cytokines. Other extracellular activators include antibodies or molecular binding entities that recognize cell surface markers or receptors including B cell receptor complex, B cell co-receptor complex or surface immunoglobulins. In one embodiment, cell surface markers, receptors or immunoglobulins are crosslinked by the activators. In a further embodiment, the crosslinking activator is a polyclonal IgM antibody, a monoclonal IgM antibody, F(ab)₂ IgM, biotinylated F(ab)₂ IgM, biotinylated polyclonal anti-IgM, or biotinylated monoclonal anti-IgM. In some embodiments, the modulator is a B cell receptor modulator. In some embodiments, the B cell receptor modulator is a B cell receptor activator.

An example of B cell receptor activator is a cross-linker of the B cell receptor complex or the B-cell co-receptor complex. In some embodiments, cross-linker is an antibody or molecular binding entity. In some embodiments, the cross-linker is an antibody. In some embodiments, the antibody is a multivalent antibody. In some embodiments, the antibody is a monovalent, bivalent, or multivalent antibody made more multivalent by attachment to a solid surface or tethered on a nanoparticle surface to increase the local valency of the epitope binding domain.

In some embodiments, the cross-linker is a molecular binding entity. In some embodiments, the molecular binding entity acts upon or binds the B cell receptor complex via carbohydrates or an epitope in the complex. In some embodiments, the molecular is a monovalent, bivalent, or multivalent is made more multivalent by attachment to a solid surface or tethered on a nanoparticle surface to increase the local valency of the epitope binding domain.

In some embodiments, the cross-linking of the B cell receptor complex or the B-cell co-receptor complex comprises binding of an antibody or molecular binding entity to the cell and then causing its crosslinking via interaction of the cell with a solid surface that causes crosslinking of the BCR complex via antibody or molecular binding entity.

In some embodiments, the crosslinker is F(ab)₂ IgM, IgG, IgD, polyclonal BCR antibodies, monoclonal BCR antibodies, or Fc receptor derived binding elements. In some embodiments, the Ig is derived from a species selected from the group consisting of mouse, goat, rabbit, pig, rat, horse, cow, shark, chicken, or llama. In some embodiments, the crosslinker is F(ab)₂ IgM, Polyclonal anti-IgM, Monoclonal anti-IgM, Biotinylated F(ab)₂ IgCM, Biotinylated Polyclonal anti-IgM, or Biotinylated Monoclonal anti-IgM.

Inhibitory modulators include inhibitors of a cellular factor or a plurality of cellular factors that participate in a cell signaling pathway. Inhibitors include a phosphatase inhibitor, such as H₂O₂, siRNA, miRNA, cantharidin, (−)-p-Bromotetramisole, Microcystin LR, Sodium Orthovanadate, Sodium Pervanadate, Vanadyl sulfate, Sodium oxodiperoxo(1,10-phenanthroline)vanadate, bis(maltolato)oxovanadium(IV), Sodium Molybdate, Sodium Permolybdate, Sodium Tartrate, Imidazole, Sodium Fluoride, β-Glycerophosphate, Sodium Pyrophosphate Decahydrate, Calyculin A, Discodermia calyx, bpV(phen), mpV(pic), DMHV, Cypermethrin, Dephostatin, Okadaic Acid, NIPP-1, N-(9,10-Dioxo-9,10-dihydro-phenanthren-2-yl)-2,2-dimethyl-propionamide, α-Bromo-4-hydroxyacetophenone, 4-Hydroxyphenacyl Br, α-Bromo-4-methoxyacetophenone, 4-Methoxyphenacyl Br, α-Bromo-4-(carboxymethoxy)acetophenone, 4-(Carboxymethoxy)phenacyl Br, and bis(4-Trifluoromethylsulfonamidophenyl)-1,4-diisopropylbenzene, phenyarsine oxide, Pyrrolidine Dithiocarbamate, or Aluminium fluoride. In some embodiments, the modulator is the phosphatase inhibitor H₂O₂.

In some embodiments, the methods of the invention provides for the use of more than one modulator. In some embodiments, the methods of the invention utilize a B cell receptor activator and a phosphatase inhibitor. In some embodiments, the methods of the invention utilize F(ab)₂IgM or biotinylated F(ab)₂IgM and H₂O₂.

Other modulators suitable for use in the invention are described in U.S. patent application Ser. Nos. 10/193,462; 10/898,734; 10/346,620; and 11/338,957, all of which are incorporated herein by reference in their entirety.

### Determination of Cell Status

After treatment with one or more modulators, if used, in some embodiments the sample is analyzed to find the activation level of an activatable element in single cells. Any suitable analysis that allows determination of the activation level of an activatable element within single cells, which provides information useful for determining the status of the individual from whom the sample was taken, may be used. Examples include flow cytometry, immunohistochemistry, immunofluorescent histochemistry with or without confocal microscopy, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, Inductively Coupled Plasma Mass Spectrometer (ICP-MS) and label-free cellular assays. Additional information for the further discrimination between single cells can be obtained by many methods known in the art including the determination of the presence of absence of extracellular and/or intracellular markers, the presence of metabolites, gene expression profiles, DNA sequence analysis, and karyotyping.

### Activatable Elements

In some embodiments, the activation level of one or more activatable elements in single cells in the sample determined. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. In some embodiments, at least about 2, 3, 4, 5, 6, 7, 8, 9, 10 or more than 10 intracellular activatable elements are determined.

Activation states of activatable elements may result from chemical additions or modifications of biomolecules and include biochemical processes such as glycosylation, phosphorylation, acetylation, methylation, biotinylation, glutamylation, glycylation, hydroxylation, isomerization, prenylation, myristoylation, lipoylation, phosphopantetheinylation, sulfation, ISGylation, nitrosylation, palmitoylation, SUMOylation, ubiquitination, neddylation, citrullination, amidation, and disulfide bond formation, disulfide bond reduction. Other possible chemical additions or modifications of biomolecules include the formation of protein carbonyls, direct modifications of protein side chains, such as o-tyrosine, chloro-, nitrotyrosine, and dityrosine, and protein adducts derived from reactions with carbohydrate and lipid derivatives. Other modifications may be non-covalent, such as binding of a ligand or binding of an allosteric modulator.

Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors.

In a further embodiment, the protein is selected from the group consisting of Erk, Syk, Zap70, Lyn, Btk, BLNK, Cbl, PLCγ2, Akt, RelA, p38, S6. In another embodiment, the protein is S6.

### Binding Element

In some embodiments of the invention, the activation state of an activatable element is determined by contacting a cell with a binding element that is specific for an activation state of the activatable element. The term “Binding element” includes any molecule, e.g., peptide, nucleic acid, small organic molecule which is capable of detecting an activation state of an activatable element over another activation state of the activatable element.

In some embodiments, the binding element is a peptide, polypeptide, oligopeptide or a protein. The peptide, polypeptide, oligopeptide or protein may be made up of naturally occurring amino acids and peptide bonds, or synthetic peptidomimetic structures. Thus “amino acid”, or “peptide residue”, as used herein include both naturally occurring and synthetic amino acids. For example, homo-phenylalanine, citrulline and noreleucine are considered amino acids for the purposes of the invention. The side chains may be in either the (R) or the (S) configuration. In some embodiments, the amino acids are in the (S) or L-configuration. If non-naturally occurring side chains are used, non-amino acid substituents may be used, for example to prevent or retard in vivo degradation. Proteins including non-naturally occurring amino acids may be synthesized or in some cases, made recombinantly; see van Hest et al., FEBS Lett 428:(1-2) 68-70 May 22, 1998 and Tang et al., Abstr. Pap Am. Chem. S218: U138 Part 2 Aug. 22, 1999, both of which are expressly incorporated by reference herein.

Methods of the present invention may be used to detect any particular activatable element in a sample that is antigenically detectable and antigenically distinguishable from other activatable element which is present in the sample. For example, as demonstrated (see, e.g., the Examples) and described herein, the activation state-specific antibodies of the present invention can be used in the present methods to identify distinct signaling cascades of a subset or subpopulation of complex cell populations; and the ordering of protein activation (e.g., kinase activation) in potential signaling hierarchies. Hence, in some embodiments the expression and phosphorylation of one or more polypeptides are detected and quantified using methods of the present invention. In some embodiments, the expression and phosphorylation of one or more polypeptides that are cellular components of a cellular pathway are detected and quantified using methods of the present invention. As used herein, the term “activation state-specific antibody” or “activation state antibody” or grammatical equivalents thereof, refer to an antibody that specifically binds to a corresponding and specific antigen. Preferably, the corresponding and specific antigen is a specific form of an activatable element. Also preferably, the binding of the activation state-specific antibody is indicative of a specific activation state of a specific activatable element.

In some embodiments, the binding element is an antibody. In some embodiment, the binding element is an activation state-specific antibody.

The term “antibody” includes full length antibodies and antibody fragments, and may refer to a natural antibody from any organism, an engineered antibody, or an antibody generated recombinantly for experimental, therapeutic, or other purposes as further defined below. Examples of antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies. The term “antibody” comprises monoclonal and polyclonal antibodies. Antibodies can be antagonists, agonists, neutralizing, inhibitory, or stimulatory.

The antibodies of the present invention may be nonhuman, chimeric, humanized, or fully human. For a description of the concepts of chimeric and humanized antibodies see Clark et al., 2000 and references cited therein (Clark, 2000, Immunol Today 21:397-402). Chimeric antibodies comprise the variable region of a nonhuman antibody, for example VH and VL domains of mouse or rat origin, operably linked to the constant region of a human antibody (see for example U.S. Pat. No. 4,816,567). In some embodiments, the antibodies of the present invention are humanized. By “humanized” antibody as used herein is meant an antibody comprising a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. Humanization relies principally on the grafting of donor CDRs onto acceptor (human) VL and VH frameworks (Winter U.S. Pat. No. 5,225,539). This strategy is referred to as “CDR grafting”. “Backmutation” of selected acceptor framework residues to the corresponding donor residues is often required to regain affinity that is lost in the initial grafted construct (U.S. Pat. No. 5,530,101; U.S. Pat. No. 5,585,089; U.S. Pat. No. 5,693,761; U.S. Pat. No. 5,693,762; U.S. Pat. No. 6,180,370; U.S. Pat. No. 5,859,205; U.S. Pat. No. 5,821,337; U.S. Pat. No. 6,054,297; U.S. Pat. No. 6,407,213). The humanized antibody optimally also will comprise at least a portion of an immunoglobulin constant region, typically that of a human immunoglobulin, and thus will typically comprise a human Fc region. Methods for humanizing non-human antibodies are well known in the art, and can be essentially performed following the method of Winter and co-workers (Jones et al., 1986, Nature 321:522-525; Riechmann et al., 1988, Nature 332:323-329; Verhoeyen et al., 1988, Science, 239:1534-1536). Additional examples of humanized murine monoclonal antibodies are also known in the art, for example antibodies binding human protein C (O'Connor et al., 1998, Protein Eng 11:321-8), interleukin 2 receptor (Queen et al., 1989, Proc Natl Acad Sci, USA 86:10029-33), and human epidermal growth factor receptor 2 (Carter et al., 1992, Proc Natl. Acad Sci USA 89:4285-9). In an alternate embodiment, the antibodies of the present invention may be fully human, that is the sequences of the antibodies are completely or substantially human. A number of methods are known in the art for generating fully human antibodies, including the use of transgenic mice (Bruggemann et al., 1997, Curr Opin Biotechnol 8:455-458) or human antibody libraries coupled with selection methods (Griffiths et al., 1998, Curr Opin Biotechnol 9:102-108).

Specifically included within the definition of “antibody” are aglycosylated antibodies. By “aglycosylated antibody” as used herein is meant an antibody that lacks carbohydrate attached at position 297 of the Fc region, wherein numbering is according to the EU system as in Kabat. The aglycosylated antibody may be a deglycosylated antibody, which is an antibody for which the Fc carbohydrate has been removed, for example chemically or enzymatically. Alternatively, the aglycosylated antibody may be a nonglycosylated or unglycosylated antibody, that is an antibody that was expressed without Fc carbohydrate, for example by mutation of one or residues that encode the glycosylation pattern or by expression in an organism that does not attach carbohydrates to proteins, for example bacteria.

As pointed out above, activation state specific antibodies can be used to detect kinase activity, however additional means for determining kinase activation are provided by the present invention. For example, substrates that are specifically recognized by protein kinases and phosphorylated thereby are known. Antibodies that specifically bind to such phosphorylated substrates but do not bind to such non-phosphorylated substrates (phospho-substrate antibodies) may be used to determine the presence of activated kinase in a sample.

In a further embodiment, an element activation profile is determined using a multiplicity of activation state antibodies that have been immobilized. Antibodies may be non-diffusibly bound to an insoluble support having isolated sample-receiving areas (e.g. a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes, and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples. In some cases magnetic beads and the like are included.

The particular manner of binding of the composition is not crucial so long as it is compatible with the reagents and overall methods of the invention, maintains the activity of the composition and is nondiffusable. Methods of binding include the use of antibodies (which do not sterically block either the ligand binding site or activation sequence when the protein is bound to the support), direct binding to “sticky” or ionic supports, chemical crosslinking, the synthesis of the antibody on the surface, etc. Following binding of the antibody, excess unbound material is removed by washing. The sample receiving areas may then be blocked through incubation with bovine serum albumin (BSA), casein or other innocuous protein or other moiety.

The antigenicity of an activated isoform of an activatable element is distinguishable from the antigenicity of non-activated isoform of an activatable element or from the antigenicity of an isoform of a different activation state. In some embodiments, an activated isoform of an element possesses an epitope that is absent in a non-activated isoform of an element, or vice versa. In some embodiments, this difference is due to covalent addition of moieties to an element, such as phosphate moieties, or due to a structural change in an element, as through protein cleavage, or due to an otherwise induced conformational change in an element which causes the element to present the same sequence in an antigenically distinguishable way. In some embodiments, such a conformational change causes an activated isoform of an element to present at least one epitope that is not present in a non-activated isoform, or to not present at least one epitope that is presented by a non-activated isoform of the element. In some embodiments, the epitopes for the distinguishing antibodies are centered around the active site of the element, although as is known in the art, conformational changes in one area of an element may cause alterations in different areas of the element as well.

Many antibodies, many of which are commercially available (for example, see Cell Signaling Technology, www.cellsignal.com, the contents which are incorporated herein by reference) have been produced which specifically bind to the phosphorylated isoform of a protein but do not specifically bind to a non-phosphorylated isoform of a protein. Many such antibodies have been produced for the study of signal transducing proteins which are reversibly phosphorylated. Particularly, many such antibodies have been produced which specifically bind to phosphorylated, activated isoforms of protein. Examples of proteins that can be analyzed with the methods described herein include, but are not limited to, kinases, HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, phosphatases, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, lipid signaling, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, cytokines, IL-2, IL-4, IL-8, IL-6, interferon γ, interferon α, cytokine regulators, suppressors of cytokine signaling (SOCs), ubiquitination enzymes, Cbl, SCF ubiquitination ligase complex, APC/C, adhesion molecules, integrins, Immunoglobulin-like adhesion molecules, selectins, cadherins, catenins, focal adhesion kinase, p130CAS, cytoskeletal/contractile proteins, fodrin, actin, paxillin, myosin, myosin binding proteins, tubulin, eg5/KSP, CENPs, heterotrimeric G proteins, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, guanine nucleotide exchange factors, Vav, Tiam, Sos, Dbl, PRK, TSC1,2, GTPase activating proteins, Ras-GAP, Arf-GAPs, Rho-GAPs, caspases, Caspase 2, Caspase 3, Caspase 6, Caspase 7, Caspase 8, Caspase 9, proteins involved in apoptosis, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, cell cycle regulators, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, vesicular transport proteins, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, isomerases, Pin1 prolyl isomerase, topoisomerases, deacetylases, Histone deacetylases, sirtuins, acetylases, histone acetylases, CBP/P300 family, MYST family, ATF2, methylases, DNA methyl transferases, demethylases, Histone H3K4 demethylases, H3K27, JHDM2A, UTX, tumor suppressor genes, VHL, WT-1, p53, Hdm, PTEN, proteases, ubiquitin proteases, urokinase-type plasminogen activator (uPA) and uPA receptor (uPAR) system, cathepsins, metalloproteinases, esterases, hydrolases, separase, ion channels, potassium channels, sodium channels, molecular transporters, multi-drug resistance proteins, P-Gycoprotein, nucleoside transporters, transcription factors/DNA binding proteins, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, regulators of translation, pS6, 4EPB-1, eIF4E-binding protein, regulators of transcription, RNA polymerase, initiation factors, elongation factors. In some embodiments, the protein is S6.

In addition to activatable elements, in some embodiments cells are classified, at least in part, based on cell surface markers. Antibodies to such markers are well-known and commercially available. For hematological pre-pathological and pathological conditions the cell surface markers of interest that may be used in the methods of the invention include CD2, CD3, CD4, CD5, CD7, CD9, CD10, CD11, CD11b, CD13, CD14, CD15, cCD15, CD19, CD20, CD21, CD22, CD23, CD24, CD31, CD33, CD34, CD36, CD37, CD38, CD39, CD40, CD43, CD44, CD45, cCD45, CD48, CD54, CD56, CD61, CD64, CD65, CD70, CD79b, CD81, CD87, CD116, CD117, CD133, CD135, CD235a, Integrinβ7, CXCR5, LAIR-1, CCR6, kappa light chain, lambda light chain, HLA-DR, MPO, LF, and TdT, and combinations thereof.

For pre-pathological and pathological solid cancer conditions, the cell surface markers of interest that may be used in the methods of the invention include, but are not limited to cell adhesion molecule (EpCAM), also known as epithelial-specific antigen (ESA), carcinoembryonic antigen (CEA), fetal oncogene platelet derived growth factor receptor (PDGFR), epidermal growth factor receptors (EGFR), Her2, Her3, Her 4, cKit, fibroblast growth factor receptor (FGFR,), insulin like growth factor 1 receptor (IGF1R,) insulin receptor (IR), vascular endothelial growth factor receptor 1, (VEGFR1), VEGFR2, VEGFR3, TIERs, Ephs, Integrin family, and cadherins.

In some embodiments, an epitope-recognizing fragment of an activation state antibody rather than the whole antibody is used. In some embodiments, the epitope-recognizing fragment is immobilized. In some embodiments, the antibody light chain that recognizes an epitope is used. A recombinant nucleic acid encoding a light chain gene product that recognizes an epitope may be used to produce such an antibody fragment by recombinant means well known in the art.

Non-activation state antibodies may also be used in the present invention. In some embodiments, non-activation state antibodies bind to epitopes in both activated and non-activated forms of an element. Such antibodies may be used to determine the amount of non-activated plus activated element in a sample. In some embodiments, non-activation state antibodies bind to epitopes present in non-activated forms of an element but absent in activated forms of an element. Such antibodies may be used to determine the amount of non-activated element in a sample. Both types of non-activation state antibodies may be used to determine if a change in the amount of activation state element, for example from samples before and after treatment with a candidate bioactive agent as described herein, coincide with changes in the amount of non-activation state element. For example, such antibodies can be used to determine whether an increase in activated element is due to activation of non-activation state element, or due to increased expression of the element, or both.

In some embodiments, antibodies are immobilized using beads analogous to those known and used for standardization in flow cytometry. Attachment of a multiplicity of activation state specific antibodies to beads may be done by methods known in the art and/or described herein. Such conjugated beads may be contacted with sample, preferably cell extract, under conditions that allow for a multiplicity of activated elements, if present, to bind to the multiplicity of immobilized antibodies. A second multiplicity of antibodies comprising non-activation state antibodies which are uniquely labeled may be added to the immobilized activation state specific antibody-activated element complex and the beads may be sorted by FACS on the basis of the presence of each label, wherein the presence of label indicates binding of corresponding second antibody and the presence of corresponding activated element.

In alternative embodiments of the instant invention, aromatic amino acids of protein binding elements may be replaced with D- or L-naphylalanine, D- or L-phenylglycine, D- or L-2-thieneylalanine, D- or L-1-, 2-, 3- or 4-pyreneylalanine, D- or L-3-thieneylalanine, D- or L-(2-pyridinyl)-alanine, D- or L-(3-pyridinyl)-alanine, D- or L-(2-pyrazinyl)-alanine, D- or L-(4-isopropyl)-phenylglycine, D-(trifluoromethyl)-phenylglycine, D-(trifluoromethyl)-phenylalanine, D-p-fluorophenylalanine, D- or L-p-biphenylphenylalanine, D- or L-p-methoxybiphenylphenylalanine, D- or L-2-indole(alkyl)alanines, and D- or L-alkylalanines where alkyl may be substituted or unsubstituted methyl, ethyl, propyl, hexyl, butyl, pentyl, isopropyl, iso-butyl, sec-isotyl, iso-pentyl, and non-acidic amino acids of C1-C20.

Acidic amino acids can be substituted with non-carboxylate amino acids while maintaining a negative charge, and derivatives or analogs thereof, such as the non-limiting examples of (phosphono)alanine, glycine, leucine, isoleucine, threonine, or serine; or sulfated (e.g., —SO3H) threonine, serine, or tyrosine.

Other substitutions may include nonnatural hydroxylated amino acids may made by combining “alkyl” with any natural amino acid. The term “alkyl” as used herein refers to a branched or unbranched saturated hydrocarbon group of 1 to 24 carbon atoms, such as methyl, ethyl, n-propyl, isoptopyl, n-butyl, isobutyl, t-butyl, octyl, decyl, tetradecyl, hexadecyl, eicosyl, tetracisyl and the like. Alkyl includes heteroalkyl, with atoms of nitrogen, oxygen and sulfur. In some embodiments, alkyl groups herein contain 1 to 12 carbon atoms. Basic amino acids may be substituted with alkyl groups at any position of the naturally occurring amino acids lysine, arginine, ornithine, citrulline, or (guanidino)-acetic acid, or other (guanidino)alkyl-acetic acids, where “alkyl” is define as above. Nitrile derivatives (e.g., containing the CN-moiety in place of COOH) may also be substituted for asparagine or glutamine, and methionine sulfoxide may be substituted for methionine. Methods of preparation of such peptide derivatives are well known to one skilled in the art.

In addition, any amide linkage in any of the polypeptides may be replaced by a ketomethylene moiety. Such derivatives are expected to have the property of increased stability to degradation by enzymes, and therefore possess advantages for the formulation of compounds which may have increased in vivo half lives, as administered by oral, intravenous, intramuscular, intraperitoneal, topical, rectal, intraocular, or other routes.

Additional amino acid modifications of amino acids of variant polypeptides of to the present invention may include the following: Cysteinyl residues may be reacted with alpha-haloacetates (and corresponding amines), such as 2-chloroacetic acid or chloroacetamide, to give carboxymethyl or carboxyamidomethyl derivatives. Cysteinyl residues may also be derivatized by reaction with compounds such as bromotrifluoroacetone, alpha-bromo-beta-(5-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole.

Histidyl residues may be derivatized by reaction with compounds such as diethylprocarbonate e.g., at pH 5.5-7.0 because this agent is relatively specific for the histidyl side chain, and para-bromophenacyl bromide may also be used; e.g., where the reaction is preferably performed in 0.1M sodium cacodylate at pH 6.0.

Lysinyl and amino terminal residues may be reacted with compounds such as succinic or other carboxylic acid anhydrides. Derivatization with these agents is expected to have the effect of reversing the charge of the lysinyl residues.

Other suitable reagents for derivatizing alpha-amino-containing residues include compounds such as imidoesters, e.g., as methyl picolinimidate; pyridoxal phosphate; pyridoxal; chloroborohydride; trinitrobenzenesulfonic acid; O-methylisourea; 2,4 pentanedione; and transaminase-catalyzed reaction with glyoxylate. Arginyl residues may be modified by reaction with one or several conventional reagents, among them phenylglyoxal, 2,3-butanedione, 1,2-cyclohexanedione, and ninhydrin according to known method steps. Derivatization of arginine residues requires that the reaction be performed in alkaline conditions because of the high pKa of the guanidine functional group. Furthermore, these reagents may react with the groups of lysine as well as the arginine epsilon-amino group. The specific modification of tyrosyl residues per se is well known, such as for introducing spectral labels into tyrosyl residues by reaction with aromatic diazonium compounds or tetranitromethane.

N-acetylimidizol and tetranitromethane may be used to form O-acetyl tyrosyl species and 3-nitro derivatives, respectively. Carboxyl side groups (aspartyl or glutamyl) may be selectively modified by reaction with carbodiimides (R′—N—C—N—R′) such as 1-cyclohexyl-3-(2-morpholiny-1-(4-ethyl) carbodiimide or 1-ethyl-3-(4-azonia-4,4-dimethylpentyl) carbodiimide. Furthermore aspartyl and glutamyl residues may be converted to asparaginyl and glutaminyl residues by reaction with ammonium ions.

Glutaminyl and asparaginyl residues may be frequently deamidated to the corresponding glutamyl and aspartyl residues. Alternatively, these residues may be deamidated under mildly acidic conditions. Either form of these residues falls within the scope of the present invention.

In some embodiments, the activation state-specific binding element is a peptide comprising a recognition structure that binds to a target structure on an activatable protein. A variety of recognition structures are well known in the art and can be made using methods known in the art, including by phage display libraries (see e.g., Gururaja et al. Chem. Biol. (2000) 7:515-27; Houimel et al., Eur. J. Immunol. (2001) 31:3535-45; Cochran et al. J. Am. Chem. Soc. (2001) 123:625-32; Houimel et al. Int. J. Cancer (2001) 92:748-55, each incorporated herein by reference). Further, fluorophores can be attached to such antibodies for use in the methods of the present invention.

A variety of recognitions structures are known in the art (e.g., Cochran et al., J. Am. Chem. Soc. (2001) 123:625-32; Boer et al., Blood (2002) 100:467-73, each expressly incorporated herein by reference)) and can be produced using methods known in the art (see e.g., Boer et al., Blood (2002) 100:467-73; Gualillo et al., Mol. Cell Endocrinol. (2002) 190:83-9, each expressly incorporated herein by reference)), including for example combinatorial chemistry methods for producing recognition structures such as polymers with affinity for a target structure on an activatable protein (see e.g., Barn et al., J. Comb. Chem. (2001) 3:534-41; Ju et al., Biotechnol. (1999) 64:232-9, each expressly incorporated herein by reference). In another embodiment, the activation state-specific antibody is a protein that only binds to an isoform of a specific activatable protein that is phosphorylated and does not bind to the isoform of this activatable protein when it is not phosphorylated or nonphosphorylated. In another embodiment the activation state-specific antibody is a protein that only binds to an isoform of an activatable protein that is intracellular and not extracellular, or vice versa. In a some embodiment, the recognition structure is an anti-laminin single-chain antibody fragment (scFv) (see e.g., Sanz et al., Gene Therapy (2002) 9:1049-53; Tse et al., J. Mol. Biol. (2002) 317:85-94, each expressly incorporated herein by reference).

In some embodiments the binding element is a nucleic acid. The term “nucleic acid” include nucleic acid analogs, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphosphoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4:395 (1994); Jeffs et al., J. Biomolecular NMR 34:17 (1994); Tetrahedron Lett. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of additional moieties such as labels, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occurring nucleic acids and analogs may be made. In some embodiments, peptide nucleic acids (PNA) which includes peptide nucleic acid analogs are used. These backbones are substantially non-ionic under neutral conditions, in contrast to the highly charged phosphodiester backbone of naturally occurring nucleic acids.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

In some embodiments, the binding element is a synthetic compound. Any numbers of techniques are available for the random and directed synthesis of a wide variety of organic compounds and biomolecules, including expression of randomized oligonucleotides. See for example WO 94/24314, hereby expressly incorporated by reference, which discusses methods for generating new compounds, including random chemistry methods as well as enzymatic methods.

Alternatively, some embodiments utilize natural compounds, as binding elements, in the form of bacterial, fungal, plant and animal extracts that are available or readily produced.

Additionally, natural or synthetically produced compounds are readily modified through conventional chemical, physical and biochemical means. Known pharmacological agents may be subjected to directed or random chemical modifications, including enzymatic modifications, to produce binding elements that may be used in the instant invention.

In some embodiment the binding element is a small organic compound. Binding elements can be synthesized from a series of substrates that can be chemically modified. “Chemically modified” herein includes traditional chemical reactions as well as enzymatic reactions. These substrates generally include, but are not limited to, alkyl groups (including alkanes, alkenes, alkynes and heteroalkyl), aryl groups (including arenes and heteroaryl), alcohols, ethers, amines, aldehydes, ketones, acids, esters, amides, cyclic compounds, heterocyclic compounds (including purines, pyrimidines, benzodiazepins, beta-lactams, tetracylines, cephalosporins, and carbohydrates), steroids (including estrogens, androgens, cortisone, ecodysone, etc.), alkaloids (including ergots, vinca, curare, pyrollizdine, and mitomycines), organometallic compounds, hetero-atom bearing compounds, amino acids, and nucleosides. Chemical (including enzymatic) reactions may be done on the moieties to form new substrates or binding elements that can then be used in the present invention.

In some embodiments the binding element is a carbohydrate. As used herein the term carbohydrate is meant to include any compound with the general formula (CH₂O)ₙ. Examples of carbohydrates are di-, tri- and oligosaccharides, as well polysaccharides such as glycogen, cellulose, and starches.

In some embodiments the binding element is a lipid. As used herein the term lipid herein is meant to include any water insoluble organic molecule that is soluble in nonpolar organic solvents. Examples of lipids are steroids, such as cholesterol, and phospholipids such as sphingomeylin.

Examples of activatable elements, activation states and methods of determining the activation state of activatable elements are described in US publication number 20060073474 entitled “Methods and compositions for detecting the activation state of multiple proteins in single cells” and US publication number 20050112700 entitled “Methods and compositions for risk stratification” the content of which are incorporate here by reference.

These and other elements are known to those of skill in the art. See U.S. patent application Ser. Nos. 10/193,462; 10/898,734; 10/346,620; and 11/338,957, all of which are incorporated herein by reference in their entirety.

### Labels

The methods and compositions of the instant invention provide binding elements comprising a label or tag. By label is meant a molecule that can be directly (i.e., a primary label) or indirectly (i.e., a secondary label) detected; for example a label can be visualized and/or measured or otherwise identified so that its presence or absence can be known. A compound can be directly or indirectly conjugated to a label which provides a detectable signal, e.g. radioisotopes, fluorescers, enzymes, antibodies, particles such as magnetic particles, chemiluminescers, or specific binding molecules, etc. Specific binding molecules include pairs, such as biotin and streptavidin, digoxin and antidigoxin etc. Examples of labels include, but are not limited to, optical fluorescent and chromogenic dyes including labels, label enzymes and radioisotopes.

In some embodiments, one or more binding elements are uniquely label. Using the example of two activation state specific antibodies, by “uniquely labeled” is meant that a first activation state antibody recognizing a first activated element comprises a first label, and second activation state antibody recognizing a second activated element comprises a second label, wherein the first and second labels are detectable and distinguishable, making the first antibody and the second antibody uniquely labeled.

In general, labels fall into four classes: a) isotopic labels, which may be radioactive or heavy isotopes; b) magnetic, electrical, thermal labels; c) colored, optical labels including luminescent, phosphorous and fluorescent dyes or moieties; and d) binding partners. Labels can also include enzymes (horseradish peroxidase, etc.) and magnetic particles. In some embodiments, the detection label is a primary label. A primary label is one that can be directly detected, such as a fluorophore.

Labels include optical labels such as fluorescent dyes or moieties. Fluorophores can be either “small molecule” fluors, or proteinaceous fluors (e.g. green fluorescent proteins and all variants thereof).

Suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, Texas Red, IAEDANS, EDANS, BODIPY FL, LC Red 640, Cy 5, Cy 5.5, LC Red 705 and Oregon green. Suitable optical dyes are described in the 1996 Molecular Probes Handbook by Richard P. Haugland, hereby expressly incorporated by reference. Suitable fluorescent labels also include, but are not limited to, green fluorescent protein (GFP; Chalfie, et al., Science 263(5148):802-805 (Feb. 11, 1994); and EGFP; Clontech—Genbank Accession Number U55762), blue fluorescent protein (BFP; 1. Quantum Biotechnologies, Inc. 1801 de Maisonneuve Blvd. West, 8th Floor, Montreal (Quebec) Canada H3H1J9; 2. Stauber, R. H. Biotechniques 24(3):462-471 (1998); 3. Heim, R. and Tsien, R. Y. Curr. Biol. 6:178-182 (1996)), enhanced yellow fluorescent protein (EYFP; 1. Clontech Laboratories, Inc., 1020 East Meadow Circle, Palo Alto, Calif. 94303), luciferase (Ichiki, et al., J. Immunol. 150(12):5408-5417 (1993)), .beta.-galactosidase (Nolan, et al., Proc Natl Acad Sci USA 85(8):2603-2607 (April 1988)) and Renilla WO 92/15673; WO 95/07463; WO 98/14605; WO 98/26277; WO 99/49019; U.S. Pat. No. 5,292,658; U.S. Pat. No. 5,418,155; U.S. Pat. No. 5,683,888; U.S. Pat. No. 5,741,668; U.S. Pat. No. 5,777,079; U.S. Pat. No. 5,804,387; U.S. Pat. No. 5,874,304; U.S. Pat. No. 5,876,995; and U.S. Pat. No. 5,925,558). All of the above-cited references are expressly incorporated herein by reference.

In some embodiments, labels for use in the present invention include: Alexa-Fluor dyes (Alexa Fluor 350, Alexa Fluor 430, Alexa Fluor 488, Alexa Fluor 546, Alexa Fluor 568, Alexa Fluor 594, Alexa Fluor 633, Alexa Fluor 660, Alexa Fluor 680), Cascade Blue, Cascade Yellow and R-phycoerythrin (PE) (Molecular Probes) (Eugene, Oreg.), FITC, Rhodamine, and Texas Red (Pierce, Rockford, Ill.), Cy5, Cy5.5, Cy7 (Amersham Life Science, Pittsburgh, Pa.). Tandem conjugate protocols for Cy5PE, Cy5.5PE, Cy7PE, Cy5.5APC, Cy7APC can be found at http://www.drmr.com/index.html. Antibodies and labels are commercially available at Becton Dickinson, http://www.bdbiosciences.com/features/products/display_product.php?keyID=94. Quantitation of fluorescent probe conjugation may be assessed to determine degree of labeling and protocols including dye spectral properties are also well known in the art.

In some embodiments, the fluorescent label is a GFP and, more preferably, a Renilla, Ptilosarcus, or Aequorea species of GFP.

In some embodiments, a secondary detectable label is used. A secondary label is one that is indirectly detected; for example, a secondary label can bind or react with a primary label for detection, can act on an additional product to generate a primary label (e.g. enzymes), etc. Secondary labels include, but are not limited to, one of a binding partner pair; chemically modifiable moieties; nuclease inhibitors, enzymes such as horseradish peroxidase, alkaline phosphatases, luciferases, etc.

In some embodiments, the secondary label is a binding partner pair. For example, the label may be a hapten or antigen, which will bind its binding partner. For example, suitable binding partner pairs include, but are not limited to: antigens (such as proteins (including peptides) and small molecules) and antibodies (including fragments thereof (FAbs, etc.)); proteins and small molecules, including biotin/streptavidin; enzymes and substrates or inhibitors; other protein-protein interacting pairs; receptor-ligands; and carbohydrates and their binding partners. Nucleic acid—nucleic acid binding proteins pairs are also useful. Binding partner pairs include, but are not limited to, biotin (or imino-biotin) and streptavidin, digeoxinin and Abs, and Prolinx™ reagents.

In some embodiments, the binding partner pair comprises an antigen and an antibody that will specifically bind to the antigen. By “specifically bind” herein is meant that the partners bind with specificity sufficient to differentiate between the pair and other components or contaminants of the system. The binding should be sufficient to remain bound under the conditions of the assay, including wash steps to remove non-specific binding. In some embodiments, the dissociation constants of the pair will be less than about 10⁻⁴ to 10⁻⁹ M⁻¹, with less than about 10⁻⁵ to 10⁻⁹ M⁻¹ being preferred and less than about 10⁻⁷ to 10⁻⁹ M⁻¹ being particularly preferred.

In some embodiment, the secondary label is a chemically modifiable moiety. In this embodiment, labels comprising reactive functional groups are incorporated into the molecule to be labeled. The functional group can then be subsequently labeled (e.g. either before or after the assay) with a primary label. Suitable functional groups include, but are not limited to, amino groups, carboxy groups, maleimide groups, oxo groups and thiol groups, with amino groups and thiol groups being particularly preferred. For example, primary labels containing amino groups can be attached to secondary labels comprising amino groups, for example using linkers as are known in the art; for example, homo- or hetero-bifunctional linkers as are well known (see 1994 Pierce Chemical Company catalog, technical section on cross-linkers, pages 155-200, incorporated herein by reference).

In some embodiments, multiple fluorescent labels are employed in the methods and compositions of the present invention. In some embodiments, each label is distinct and distinguishable from other labels.

As will be appreciated in the art antibody-label conjugation may be performed using standard procedures or by using protein-protein/protein-dye crosslinking kits from Molecular Probes (Eugene, Oreg.).

In some embodiments, labeled antibodies are used for functional analysis of activatable proteins in cells. In performing such analysis several areas of the experiment are considered: (1) identification of the proper combination of antibody cocktails for the stains (2), identification of the sequential procedure for the staining using the antigens (i.e., the activatable protein) and antibody clones of interest, and (3) thorough evaluation of cell culture conditions' effect on cell stimulation. Antigen clone selection is of particular importance for surface antigens of human cells, as different antibody clones yield different result and do not stain similarly in different protocols. Selection of cell types and optimization of culture conditions is also a critical component in detecting differences. For example, some cell lines have the ability to adapt to culture conditions and can yield heterogeneous responses.

Alternatively, detection systems based on FRET, discussed in detail below, may be used. FRET finds use in the instant invention, for example, in detecting activation states that involve clustering or multimerization wherein the proximity of two FRET labels is altered due to activation. In some embodiments, at least two fluorescent labels are used which are members of a fluorescence resonance energy transfer (FRET) pair.

FRET is phenomenon known in the art wherein excitation of one fluorescent dye is transferred to another without emission of a photon. A FRET pair consists of a donor fluorophore and an acceptor fluorophore. The fluorescence emission spectrum of the donor and the fluorescence absorption spectrum of the acceptor must overlap, and the two molecules must be in close proximity. The distance between donor and acceptor at which 50% of donors are deactivated (transfer energy to the acceptor) is defined by the Forster radius (Ro), which is typically 10-100 Å. Changes in the fluorescence emission spectrum comprising FRET pairs can be detected, indicating changes in the number of that are in close proximity (i.e., within 100 521 of each other). This will typically result from the binding or dissociation of two molecules, one of which is labeled with a FRET donor and the other of which is labeled with a FRET acceptor, wherein such binding brings the FRET pair in close proximity. Binding of such molecules will result in an increased fluorescence emission of the acceptor and/or quenching of the fluorescence emission of the donor.

FRET pairs (donor/acceptor) useful in the invention include, but are not limited to, EDANS/fluorescein, IAEDANS/fluorescein, fluorescein/tetramethylrhodamine, fluorescein/LC Red 640, fluorescein/Cy 5, fluorescein/Cy 5.5 and fluorescein/LC Red 705.

In some embodiments when FRET is used, a fluorescent donor molecule and a nonfluorescent acceptor molecule (“quencher”) may be employed. In this application, fluorescent emission of the donor will increase when quencher is displaced from close proximity to the donor and fluorescent emission will decrease when the quencher is brought into close proximity to the donor. Useful quenchers include, but are not limited to, TAMRA, DABCYL, QSY 7 and QSY 33. Useful fluorescent donor/quencher pairs include, but are not limited to EDANS/DABCYL, Texas Red/DABCYL, BODIPY/DABCYL, Lucifer yellow/DABCYL, coumarin/DABCYL and fluorescein/QSY 7 dye.

The skilled artisan will appreciate that FRET and fluorescence quenching allow for monitoring of binding of labeled molecules over time, providing continuous information regarding the time course of binding reactions.

Preferably, changes in the degree of FRET are determined as a function of the change in the ratio of the amount of fluorescence from the donor and acceptor moieties, a process referred to as “ratioing.” Changes in the absolute amount of substrate, excitation intensity, and turbidity or other background absorbances in the sample at the excitation wavelength affect the intensities of fluorescence from both the donor and acceptor approximately in parallel. Therefore the ratio of the two emission intensities is a more robust and preferred measure of cleavage than either intensity alone.

The ratio-metric fluorescent reporter system described herein has significant advantages over existing reporters for protein integration analysis, as it allows sensitive detection and isolation of both expressing and non-expressing single living cells. In some embodiments, the assay system uses a non-toxic, non-polar fluorescent substrate that is easily loaded and then trapped intracellularly. Modification of the fluorescent substrate by a cognate protein yields a fluorescent emission shift as substrate is converted to product. Because the reporter readout is ratiometric it is unique among reporter protein assays in that it controls for variables such as the amount of substrate loaded into individual cells. The stable, easily detected, intracellular readout eliminates the need for establishing clonal cell lines prior to expression analysis. This system and other analogous flow sorting systems can be used to isolate cells having a particular receptor element clustering and/or activation profile from pools of millions of viable cells.

The methods and composition of the present invention may also make use of label enzymes. By label enzyme is meant an enzyme that may be reacted in the presence of a label enzyme substrate that produces a detectable product. Suitable label enzymes for use in the present invention include but are not limited to, horseradish peroxidase, alkaline phosphatase and glucose oxidase. Methods for the use of such substrates are well known in the art. The presence of the label enzyme is generally revealed through the enzyme's catalysis of a reaction with a label enzyme substrate, producing an identifiable product. Such products may be opaque, such as the reaction of horseradish peroxidase with tetramethyl benzedine, and may have a variety of colors. Other label enzyme substrates, such as Luminol (available from Pierce Chemical Co.), have been developed that produce fluorescent reaction products. Methods for identifying label enzymes with label enzyme substrates are well known in the art and many commercial kits are available. Examples and methods for the use of various label enzymes are described in Savage et al., Previews 247:6-9 (1998), Young, J. Virol. Methods 24:227-236 (1989), which are each hereby incorporated by reference in their entirety.

By radioisotope is meant any radioactive molecule. Suitable radioisotopes for use in the invention include, but are not limited to ¹⁴C, ³H, ³²P, ³³p, ³⁵S, ¹²⁵I, and ¹³¹I. The use of radioisotopes as labels is well known in the art.

As mentioned, labels may be indirectly detected, that is, the tag is a partner of a binding pair. By “partner of a binding pair” is meant one of a first and a second moiety, wherein the first and the second moiety have a specific binding affinity for each other. Suitable binding pairs for use in the invention include, but are not limited to, antigens/antibodies (for example, digoxigenin/anti-digoxigenin, dinitrophenyl (DNP)/anti-DNP, dansyl-X-anti-dansyl, Fluorescein/anti-fluorescein, lucifer yellow/anti-lucifer yellow, and rhodamine anti-rhodamine), biotin/avidin (or biotin/streptavidin) and calmodulin binding protein (CBP)/calmodulin. Other suitable binding pairs include polypeptides such as the FLAG-peptide [Hopp et al., BioTechnology, 6:1204-1210 (1988)]; the KT3 epitope peptide [Martin et al., Science, 255: 192-194 (1992)]; tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)] and the antibodies each thereto. As will be appreciated by those in the art, binding pair partners may be used in applications other than for labeling, as is described herein.

As will be appreciated by those in the art, a partner of one binding pair may also be a partner of another binding pair. For example, an antigen (first moiety) may bind to a first antibody (second moiety) that may, in turn, be an antigen for a second antibody (third moiety). It will be further appreciated that such a circumstance allows indirect binding of a first moiety and a third moiety via an intermediary second moiety that is a binding pair partner to each.

As will be appreciated by those in the art, a partner of a binding pair may comprise a label, as described above. It will further be appreciated that this allows for a tag to be indirectly labeled upon the binding of a binding partner comprising a label. Attaching a label to a tag that is a partner of a binding pair, as just described, is referred to herein as “indirect labeling”.

By “surface substrate binding molecule” or “attachment tag” and grammatical equivalents thereof is meant a molecule have binding affinity for a specific surface substrate, which substrate is generally a member of a binding pair applied, incorporated or otherwise attached to a surface. Suitable surface substrate binding molecules and their surface substrates include, but are not limited to poly-histidine (poly-his) or poly-histidine-glycine (poly-his-gly) tags and Nickel substrate; the Glutathione-S Transferase tag and its antibody substrate (available from Pierce Chemical); the flu HA tag polypeptide and its antibody 12CA5 substrate [Field et al., Mol. Cell. Biol., 8:2159-2165 (1988)]; the c-myc tag and the 8F9, 3C7, 6E10, G4, B7 and 9E10 antibody substrates thereto [Evan et al., Molecular and Cellular Biology, 5:3610-3616 (1985)]; and the Herpes Simplex virus glycoprotein D (gD) tag and its antibody substrate [Paborsky et al., Protein Engineering, 3(6):547-553 (1990)]. In general, surface binding substrate molecules useful in the present invention include, but are not limited to, polyhistidine structures (His-tags) that bind nickel substrates, antigens that bind to surface substrates comprising antibody, haptens that bind to avidin substrate (e.g., biotin) and CBP that binds to surface substrate comprising calmodulin.

Production of antibody-embedded substrates is well known; see Slinkin et al., Bioconj. Chem., 2:342-348 (1991); Torchilin et al., supra; Trubetskoy et al., Bioconj. Chem. 3:323-327 (1992); King et al., Cancer Res. 54:6176-6185 (1994); and Wilbur et al., Bioconjugate Chem. 5:220-235 (1994) (all of which are hereby expressly incorporated by reference), and attachment of or production of proteins with antigens is described above. Calmodulin-embedded substrates are commercially available, and production of proteins with CBP is described in Simcox et al., Strategies 8:40-43 (1995), which is hereby incorporated by reference in its entirety.

As will be appreciated by those in the art, tag-components of the invention can be made in various ways, depending largely upon the form of the tag. Components of the invention and tags are preferably attached by a covalent bond.

The production of tag-polypeptides by recombinant means when the tag is also a polypeptide is described below. Production of tag-labeled proteins is well known in the art and kits for such production are commercially available (for example, from Kodak and Sigma). Examples of tag labeled proteins include, but are not limited to, a Flag-polypeptide and His-polypeptide. Methods for the production and use of tag-labeled proteins are found, for example, in Winston et al., Genes and Devel. 13:270-283 (1999), incorporated herein in its entirety, as well as product handbooks provided with the above-mentioned kits.

Biotinylation of target molecules and substrates is well known, for example, a large number of biotinylation agents are known, including amine-reactive and thiol-reactive agents, for the biotinylation of proteins, nucleic acids, carbohydrates, carboxylic acids; see chapter 4, Molecular Probes Catalog, Haugland, 6th Ed. 1996, hereby incorporated by reference. A biotinylated substrate can be attached to a biotinylated component via avidin or streptavidin. Similarly, a large number of haptenylation reagents are also known (Id.).

Methods for labeling of proteins with radioisotopes are known in the art. For example, such methods are found in Ohta et al., Molec. Cell 3:535-541 (1999), which is hereby incorporated by reference in its entirety.

Production of proteins having tags by recombinant means is well known, and kits for producing such proteins are commercially available. For example, such a kit and its use are described in the QIAexpress Handbook from Qiagen by Joanne Crowe et al., hereby expressly incorporated by reference.

The functionalization of labels with chemically reactive groups such as thiols, amines, carboxyls, etc. is generally known in the art. In some embodiments, the tag is functionalized to facilitate covalent attachment. The covalent attachment of the tag may be either direct or via a linker. In one embodiment, the linker is a relatively short coupling moiety, which is used to attach the molecules. A coupling moiety may be synthesized directly onto a component of the invention and contains at least one functional group to facilitate attachment of the tag. Alternatively, the coupling moiety may have at least two functional groups, which are used to attach a functionalized component to a functionalized tag, for example. In an additional embodiment, the linker is a polymer. In this embodiment, covalent attachment is accomplished either directly, or through the use of coupling moieties from the component or tag to the polymer. In some embodiments, the covalent attachment is direct, that is, no linker is used. In this embodiment, the component preferably contains a functional group such as a carboxylic acid that is used for direct attachment to the functionalized tag. It should be understood that the component and tag may be attached in a variety of ways, including those listed above. In some embodiments, the tag is attached to the amino or carboxl terminus of the polypeptide. As will be appreciated by those in the art, the above description of the covalent attachment of a label applies to the attachment of virtually any two molecules of the present disclosure.

In some embodiments, the tag is functionalized to facilitate covalent attachment, as is generally outlined above. Thus, a wide variety of tags are commercially available which contain functional groups, including, but not limited to, isothiocyanate groups, amino groups, haloacetyl groups, maleimides, succinimidyl esters, and sulfonyl halides, all of which may be used to covalently attach the tag to a second molecule, as is described herein. The choice of the functional group of the tag will depend on the site of attachment to either a linker, as outlined above or a component of the invention. Thus, for example, for direct linkage to a carboxylic acid group of a protein, amino modified or hydrazine modified tags will be used for coupling via carbodiimide chemistry, for example using 1-ethyl-3-(3-dimethylaminopropyl)-carbodiimi-de (EDAC) as is known in the art (see Set 9 and Set 11 of the Molecular Probes Catalog, supra; see also the Pierce 1994 Catalog and Handbook, pages T-155 to T-200, both of which are hereby incorporated by reference). In one embodiment, the carbodiimide is first attached to the tag, such as is commercially available for many of the tags described herein.

### Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention can be performed according to standard techniques and protocols well-established in the art.

One or more activatable elements can be detected and/or quantified by any method that detect and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters.

In some embodiments, the present invention provides methods for determining an activatable element's activation profile for a single cell. The methods may comprise analyzing cells by flow cytometry on the basis of the activation state of at least two activatable elements. Binding elements (e.g. activation state-specific antibodies) are used to analyze cells on the basis of activatable element activation state, and can be detected as described below. Alternatively, non-binding elements systems as described above can be used in any system described herein.

When using fluorescent labeled components in the methods and compositions of the present invention, it will recognized that different types of fluorescent monitoring systems, e.g., FACS systems, can be used to practice the invention. In some embodiments, FACS systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Activation state-specific antibodies can also be labeled with quantum dots as disclosed by Chattopadhyay, P. K. et al. Quantum dot semiconductor nanocrystals for immunophenotyping by polychromatic flow cytometry. Nat. Med. 12, 972-977 (2006). Quantum dot labels are commercially available through Invitrogen, http://probes.invitrogen.com/products/qdot/.

Quantum dot labeled antibodies can be used alone or they can be employed in conjunction with organic fluorochrome conjugated antibodies to increase the total number of labels available. As the number of labeled antibodies increase so does the ability for subtyping known cell populations. Additionally, activation state-specific antibodies can be labeled using chelated or caged lanthanides as disclosed by Erkki, J. et al. Lanthanide chelates as new fluorochrome labels for cytochemistry. J. Histochemistry Cytochemistry, 36:1449-1451, 1988, and U.S. Pat. No. 7,018,850, entitled Salicylamide-Lanthanide Complexes for Use as Luminescent Markers. Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy.

In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., WO99/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation state in an activatable element in response to a modulator.

In some embodiments, the cells are first contacted with fluorescent-labeled activation state-specific binding elements (e.g. antibodies) directed against specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic filed while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyze the population of cells by flow cytometry. Preferably, cells are analyzed on the basis of the activation state of at least two activatable elements. In some embodiments, a multiplicity of activatable element activation-state antibodies is used to simultaneously determine the activation state of a multiplicity of elements.

In some embodiment, cell analysis by flow cytometry on the basis of the activation state of at least two elements is combined with a determination of other flow cytometry readable outputs, such as the presence of surface markers, granularity and cell size to provide a correlation between the activation state of a multiplicity of elements and other cell qualities measurable by flow cytometry for single cells.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation state of a cell or cell population with a control at a single time point, or by comparing cells at multiple time points to observe subpopulations arising out of the others.

The present invention provides a valuable method of determining the presence of cellular subsets within cellular populations. Ideally, signal transduction pathways are evaluated in homogeneous cell populations to ensure that variances in signaling between cells do not qualitatively nor quantitatively mask signal transduction events and alterations therein. As the ultimate homogeneous system is the single cell, the present invention allows the individual evaluation of cells to allow true differences to be identified in a significant way.

Thus, the invention provides methods of distinguishing cellular subsets within a larger cellular population. As outlined herein, these cellular subsets often exhibit altered biological characteristics (e.g. activation states, altered response to modulators) as compared to other subsets within the population. For example, as outlined herein, the methods of the invention allow the identification of subsets of cells from a population such as primary cell populations, e.g. peripheral blood mononuclear cells that exhibit altered responses (e.g. response associated with presence of a condition) as compared to other subsets. In addition, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such a peripheral blood mononuclear cells, or naive and memory lymphocytes.

When necessary, cells are dispersed into a single cell suspension, e.g. by enzymatic digestion with a suitable protease, e.g. collagenase, dispase, etc; and the like. An appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 min in acetone at −200° C.; and the like as known in the art and according to the methods described herein.

In some embodiments, one or more cells are contained in a well of a 96 well plate or other commercially available multiwell plate. In an alternate embodiment, the reaction mixture or cells are in a FACS machine. Other multiwell plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation state or activity of an activatable element, or modulation of such activation state or activity, may be sequential or in a predetermined order or grouping under conditions appropriate for the activity that is assayed for. Such conditions are described here and known in the art. Moreover, further guidance is provided below (see, e.g., in the Examples).

As will be appreciated by one of skill in the art, the instant methods and compositions find use in a variety of other assay formats in addition to flow cytometry analysis. For example, a chip analogous to a DNA chip can be used in the methods of the present invention. Arrayers and methods for spotting nucleic acid to a chip in a prefigured array are known. In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, a chip comprises a multiplicity of the “second set binding elements,” in this case generally unlabeled. Such a chip is contacted with sample, preferably cell extract, and a second multiplicity of binding elements comprising element activation state specific binding elements is used in the sandwich assay to simultaneously determine the presence of a multiplicity of activated elements in sample. Preferably, each of the multiplicity of activation state-specific binding elements is uniquely labeled to facilitate detection.

In some embodiments confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

In some embodiments, the activation level of an activatable element is measured using Inductively Coupled Plasma Mass Spectrometer (ICP-MS). A binding element that has been labeled with a specific element binds to the activatable element. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, (2007), 62(3):188-195.).

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiment, the methods of the invention include the use of liquid handling components. The liquid handling systems can include robotic systems comprising any number of components. In addition, any or all of the steps outlined herein may be automated; thus, for example, the systems may be completely or partially automated.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G. nucleotide resins or coatings, and other affinity matrix are useful in this invention.

In some embodiments, platforms for multi-well plates, multi-tubes, holders, cartridges, minitubes, deep-well plates, microfuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

### Gating

In another embodiment, a user may analyze the signaling in subpopulations based on surface markers. For example, the user could look at: “stem cell populations” by CD34+ CD38− or CD34+ CD33− expressing cells; drug transporter positive cells; e.g. P—P-glycoprotein positive cells; or multiple leukemic subclones based on CD33, CD45, HLA-DR, CD11b and analyzing signaling in each subpopulation. In another alternative embodiment, a user may analyze the data based on intracellular markers, such as transcription factors or other intracellular proteins; based on a functional assay (e.g., dye efflux assay to determine drug transporter+cells or fluorescent glucose uptake) or based on other fluorescent markers. In some embodiments, gates are used to identify the presence of specific subpopulations in existing independent data. The existing independent data can be data stored in a computer from a previous patient, or data from independent studies using different patients.

In some embodiments where flow cytometry is used, prior to analyzing of data the populations of interest and the method for characterizing these populations are determined. For instance, there are at least two general ways of identifying populations for data analysis: (i) “Outside-in” comparison of Parameter sets for individual samples or subset (e.g., patients in a trial). In this more common case, cell populations are homogenous or lineage gated in such a way as to create distinct sets considered to be homogenous for targets of interest. An example of sample-level comparison would be the identification of signaling profiles in tumor cells of a patient and correlation of these profiles with non-random distribution of clinical responses. This is considered an outside-in approach because the population of interest is pre-defined prior to the mapping and comparison of its profile to other populations. (ii) “Inside-out” comparison of Parameters at the level of individual cells in a heterogeneous population. An example of this would be the signal transduction state mapping of mixed hematopoietic cells under certain conditions and subsequent comparison of computationally identified cell clusters with lineage specific markers. This could be considered an inside-out approach to single cell studies as it does not presume the existence of specific populations prior to classification. A major drawback of this approach is that it creates populations which, at least initially, require multiple transient markers to enumerate and may never be accessible with a single cell surface epitope. As a result, the biological significance of such populations can be difficult to determine. The main advantage of this unconventional approach is the unbiased tracking of cell populations without drawing potentially arbitrary distinctions between lineages or cell types.

Each of these techniques capitalizes on the ability of flow cytometry to deliver large amounts of multiparameter data at the single cell level. For cells associated with a condition (e.g. neoplastic or hematopoetic condition), a third “meta-level” of data exists because cells associated with a condition (e.g. cancer cells) are generally treated as a single entity and classified according to historical techniques. These techniques have included organ or tissue of origin, degree of differentiation, proliferation index, metastatic spread, and genetic or metabolic data regarding the patient.

In some embodiments, the present invention uses variance mapping techniques for mapping condition signaling space. These methods represent a significant advance in the study of condition biology because it enables comparison of conditions independent of a putative normal control. Traditional differential state analysis methods (e.g., DNA microarrays, subtractive Northern blotting) generally rely on the comparison of cells associated with a condition from each patient sample with a normal control, generally adjacent and theoretically untransformed tissue. Alternatively, they rely on multiple clusterings and reclusterings to group and then further stratify patient samples according to phenotype. In contrast, variance mapping of condition states compares condition samples first with themselves and then against the parent condition population. As a result, activation states with the most diversity among conditions provide the core parameters in the differential state analysis. Given a pool of diverse conditions, this technique allows a researcher to identify the molecular events that underlie differential condition pathology (e.g., cancer responses to chemotherapy), as opposed to differences between conditions and a proposed normal control.

In some embodiments, when variance mapping is used to profile the signaling space of patient samples, conditions whose signaling response to modulators is similar are grouped together, regardless of tissue or cell type of origin. Similarly, two conditions (e.g. two tumors) that are thought to be relatively alike based on lineage markers or tissue of origin could have vastly different abilities to interpret environmental stimuli and would be profiled in two different groups.

When groups of signaling profiles have been identified it is frequently useful to determine whether other factors, such as clinical responses, presence of gene mutations, and protein expression levels, are non-randomly distributed within the groups. If experiments or literature suggest such a hypothesis in an arrayed flow cytometry experiment, it can be judged with simple statistical tests, such as the Student's t-test and the X² test. Similarly, if two variable factors within the experiment are thought to be related, the r² correlation coefficient from a linear regression is used to represent the degree of this relationship.

### Classes of Cells

The activation state of an individual activatable element is either in the on or off state. As an illustrative example, an individual phosphorylatable site on a protein will either be phosphorylated and then be in the “on” state or it will not be phosphorylated and hence, it will be in the “off” state. The terms “on” and “off,” when applied to an activatable element that is a part of a cellular constituent, are used here to describe the state of the activatable element (e.g., phosphorylated is “on” and non-phosphorylated is “off”), and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis for classifying cells is that the distribution of activation levels for one or more specific activatable elements will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to classify cells in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may not contain activatable elements, of cell or a population of cells may be used to classify a cell or a population of cells into a class.

Once the activation level of intracellular activatable elements of individual single cells is known they can be placed into one or more classes. In some embodiments, cells are placed in predefined classes. A predefined class encompasses a class of cells wherein every cell has the same or substantially the same known activation level, or range of activation levels, of one or more intracellular activatable elements. For example, if the activation levels of five intracellular activatable elements are analyzed, predefined classes that encompass one or more of the intracellular activatable elements can be constructed based on the activation level, or ranges of the activation levels, of each of these five elements. It is understood that activation levels can exist as a distribution and that an activation level of a particular element used to classify a cell may be a particular point on the distribution but more typically may be a portion of the distribution.

In addition to activation levels of intracellular activatable elements, expression levels of intracellular or extracellular biomolecules, e.g., proteins, may be used alone or in combination with activation states of activatable elements to classify cells. Further, additional cellular elements, e.g., biomolecules or molecular complexes such as RNA, DNA, carbohydrates, metabolites, and the like, may be used in conjunction with activatable states or expression levels in the classification of cells encompassed here.

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

A predefined class of cells, additionally, may be further divided into subsets that are themselves predefined classes based on other factors, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. For example, if B cells represent a predefined class, they can be further subdivided based on the expression of cell surface markers such as CD19, CD20, or CD22.

Alternatively, predefined classes of cells can be aggregated based upon shared characteristics that may include inclusion in one or more additional predefined class or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus or other distinguishing characteristics.

The absence of a class is itself a predefined class; e.g., cells in a sample may be classified as those belonging to a class and those not belonging to that class, where the latter is itself considered a class. This is useful when it is desired to determine what the percentage of the total number of cells belong to one particular class.

The predefined classes may be determined empirically based on data from individuals that indicates status, e.g., health status. E.g., blood samples from the clinic and/or from clinical trials may be analyzed retrospectively to determine classes of cells; certain classes or quantitative features of the classes may be associated with certain known outcomes for the patients. For example, blood samples may be obtained from cancer patients over the course of treatment. Various outcomes, from complete remission for a number of years, to death from cancer or cancer recurrence after treatment, may be recorded. Profiles of the states of activatable elements in single cells, with or without modulator, may be obtained from retrospective samples to determine classes of cells present in the samples, numbers of cells in each class, relative numbers of class vs. class, and the like. These classes are “predefined” classes as that term is used herein, and the classes, together with their predictive value for various health statuses, may be placed in a database that is then used for analysis of further samples. As more samples are obtained and correlated health status determined, the database may be modified.

Thus, in some embodiments, the invention encompasses a database of classes of cells, where the cells are classified at least in part according to the activation level of one or more activatable elements, and clinical outcomes for patients from whom the cells are derived. Such a database may be on a computer-readable medium.

a. Rare Cells

In some embodiments, the cells are classified into a class that is considered a class of rare cells. In some embodiments, the presence of rare cell populations is used to make a diagnosis, prognosis or to predict response to a treatment. The term “rare” as used herein is used to denote a low numbers of abundance, uncommon, or scarce cells. It is contemplated that the detection of rare cell populations can be used to predict changes in health status.

In some embodiments, the cells are classified as rare cells at least in part according to the activation level of one or more activatable elements. The term “rare” as used herein designates cells of interest that are to be detected. This term is not intended to limit the relative abundances of the designated cell types, although it is preferable for the rare cells to have a relative abundance of less the 25%, 10%, 5%, 1%, 0.5%, and less.

Whether a particular cell is a rare cell can be viewed different ways. In a first manner of characterizing a cell as rare, the rare cell can be said to be any cell that does not naturally occur as a significant fraction of a given sample. For example, for human or mammalian blood, a rare cell may be any cell other than a subject's blood cell (such as a normal red blood cell and a normal white blood cell). In this view, cancer or other cells present in the blood would be considered rare cells. In addition, infiltrating cancer cells in a tissue should be considered rare cells. A second manner of characterizing a cell as rare might take into account the frequency with which that cell appears in a sample or the frequency with respect to other cells. A cell can be considered rare when the frequency of the cell is compared to more than one class of cells. When the rare cells are associated with a pathological state such as cancer, the frequency of the rare cell population can be compared to normal cells or to other cells associated with the pathological state. For example, a rare cell may be a cell that appears at a frequency of approximately 1 to 50 cells per ml of blood. A rare cell may be present in a sample, blood or tissue in a concentration of less than 1 in 10,000 cells, 1 in 100,000 cells, 1 in 1,000,000 cells, 1 in 10,000,000 cells, 1 in 100,000,000 cells, or 1 in 1,000,000,000 cells. Alternatively, rare cell frequency within a given population containing non-rare cells or other rare cells can include, but is not limited to, frequencies of less than about 1 cell in 100 cells; 1 cell in 1,000 cells; 1 cell in 10,000 cells; 1 cell in 100,000 cells; 1 cell in 1,000,000 cells; 1 cell in 10,000,000 cells; 1 cell in 100,000,000 cells; or 1 cell in 1,000,000,000 cells.

In a third manner of characterizing a cell as rare, the rare cell can be said to be a cell located at a different position when compared to normal cells in a contour or density plot. The contour or density plot represents the number of cells that share a characteristic such as the activation level of activatable proteins in response to a modulator. For example, when referring to activation levels of activatable elements in response to one or more modulator, normal individuals and patients with a pathological state might show populations with increased activation levels in response to the one or more modulators. However, the number of cells that have a specific activation level (e.g. specific amount of an activatable element) might be different between normal individuals and patients with a pathological state. Thus, a rare cell is a cell that is within a given region in the contour or density plot that is different from the regions of normal cells. Rare cell frequency when compared to different regions containing non-rare cells or other rare cells can include, but is not limited to, a frequency of less than about 1 cell in 10 cells, 1 cell in 20 cells, 1 cell in 50 cells, 1 cell in 100 cells, 1 cell in 1,000 cells, 1 cell in 100,000 cells; or 1 cell in 1,000,000 cells. The frequency of rare cells within a region can be determined by using mathematical estimates of the centers of the contour or density plot, densities within the blobs in a plots, or the relative position of each blob in the plot to each other in N-space define the placements. For example, the frequency of the rare cell population within a region can be determined by using an eigenvector approach. Another way to calculate the frequency of the rare cell population within a region is to describe the surface of the density and calculate the differences in the volumes (e.g. how much does one shape protrude from the other). In some embodiments, the individual status of an individual (e.g. clinical outcome) is determined when the number of rare cells within a region is higher that a threshold number. In some instances, the threshold number is 0 and the finding of 1 rare cell within a region would indicate of a status of the individual (e.g. a cancer cell is present and treatment must begin). In other instances, the threshold number is 1. In still other instances, the threshold number is 2, 3, 4, 5, 6, 7, 8, 9, 10, 15, 20, 30, 40, 50, 60, 70, 80, 90, 100, 150, 200, 300, 400, 500, 600, 700, 800, 900, or 1000 cells.

The methods of the present invention allows for the determination of the status of an individual (e.g. a clinical outcome) by detecting rare cells at lower relative abundances. For example, a diagnosis can be made in a patient by detecting a rare population of cells associated with a pathological state such as cancer. In some embodiments, the status of an individual (e.g. a clinical outcome) can be determined when the number of rare cells is fewer than 10⁻² to 10⁻⁴ cells (one rare cell in 100 to 10,000 total cells). For example, the presence of 1×10⁻², 1×10⁻³, 2×10⁻³, 3×10⁻³, 4×10⁻³, 5×10⁻³, 6×10⁻³, 7×10⁻³, 8×10⁻³, 9×10⁻³, 10×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ rare cells is used to determine the status of an individual (e.g. a clinical outcome such as probability of relapse). In some embodiments, the number of rare cells used to determine the status of an individual is fewer than 1×10⁻². In some embodiments, the number of rare cells used to determine the status of an individual is fewer than 5×10⁻⁴. In some embodiments, the number of rare cells used to determine the status of an individual is fewer than 4.5×10⁻⁴. In some embodiments, the number of rare cells used to determine the status of an individual is fewer than 4×10⁻⁴. In some embodiments, the number of rare cells used to determine the status of an individual is fewer than 3.5×10⁻⁴. In some embodiments, the number of rare cells used to determine the status of an individual is fewer than 3.5×10⁻⁴. In some embodiments, the number of rare cells used to determine the status of an individual is fewer than 2×10⁻⁴.

In some embodiments, the methods describe herein provide for tracking the emergence and/or disappearance of rare cell populations. In some embodiments, the methods described herein provides for the determination of the presence or absence of pre-existing populations of rare cells as is the case when a patient is originally diagnosed with a condition such as cancer. These pre-existing cells can be from a single clone of cells or multiple clones. In some embodiments, the methods described herein provides for the determination for the presence or absence of rare cells population that develops over time such as a rare cell population that develops over the course of a treatment. These later developed cells can be from a single clone of cells or multiple clones. Thus, in some embodiments, the methods described herein provide for the determination of one or more rare cell population at diagnosis, during treatment and after treatment. The methods described herein provide for the monitoring of a patient at several stages, thus, allowing for example the identification of rare cells populations that have responded to treatment, rare cell population that did not respond and/or rare cells populations that emerge during the course of treatment or during remission stages. The determination of rare cells populations allows for very sensitive detection of changes in the health status of an individual, which allows for early diagnosis and/or treatment.

The methods of the present invention allows for the determination of the status of an individual (e.g. a clinical outcome) by detecting rare cells that are strongly associated with said status. In some embodiments, the p value in the analysis is below 0.05, 04, 0.03, 0.02, 0.01, 0.009, 0.005, or 0.001. In some embodiments, the p value is below 0.001. Thus in some embodiments, the status of an individual can be determined by detecting rare cells wherein the p value is below 0.05, 04, 0.03, 0.02, 0.01, 0.009, 0.005, or 0.001. In some embodiments, the p value is below 0.001. In some embodiments, the status of an individual can be determined by detecting rare cells wherein the AUC value is higher than 0.5, 0.6, 07, 0.8 or 0.9. In some embodiments, the status of an individual can be determined by detecting rare cells wherein the AUC value is higher than 0.7. In some embodiments, the status of an individual can be determined by detecting rare cells wherein the AUC value is higher than 0.8. In some embodiments, the status of an individual can be determined by detecting rare cells wherein the AUC value is higher than 0.9.

### Quantitative Analysis of Predefined Classes

Once a sufficient number of single cells have been placed into classes of cells (e.g. predefined classes of cells), the status of an individual (e.g. health status) can be determined by performing a quantitative analysis on one or more the predefined classes of cells. In some embodiments, the minimum number of single cells in a plurality of cells that is examined in order to determine an individual's health status is about 10, 100, 1,000, 2,500, 5,000, 10,000, 50,000, 100,000, 500,000, 1,000,000, 2,500,000, 5,000,000, or 10,000,000 cells. In some examples, the method of the present invention can be used to detect less than 200 cells in a sample for determining a health status of an individual.

In some embodiments, the maximum number of single cells in a plurality of cells that is examined in order to determine an individual's health status is about 10, 100, 1,000, 2,500, 5,000, 10,000, 50,000, 100,000, 500,000, 1,000,000, 2,500,000, 5,000,000, or 10,000,000 cells.

Any suitable method of quantitative analysis can be used including, but not limited to quantifying the number of cells in a particular class, determining if the number of cells in a particular predefined class is greater than, equal to, or less than a threshold number, determining the ratio of number of cells in one or more predefined classes to number of cells in one or more other predefined classes, determining the if the ratio of one or more predefined classes of compared to one or more other predefined classes of cells is greater than, equal to or less than a threshold number. If sequential samples are obtained, then determinations of the rate of change in the number of cells in predefined classes or ratios of numbers of cells can be calculated.

In the simplest quantitative analysis, the number of cells in one or more classes is compared to a threshold number, where if the number of cells in the predefined class is greater than, equal to, or less than the threshold number, the status of the individual may be determined.

In certain instances, the finding of 0 cells in a predefined class may be determinative as to an individual's status. In this case, the threshold number is 1, and finding fewer than one cell is indicative of the status of the individual. For example, if a predefined class of cells is associated with the presence or recurrence of a disease, for example, cancer, then the finding of 0 cells in the predefined class of cells provides evidence that the individual does not have the disease or has not experienced a recurrence.

In some embodiments, the presence of 1 cell in a predefined class may be determinative of an individual's status. In this case, the threshold number is 0, and finding even a single cell (more than zero) is indicative of the status of the individual. In an individual with high risk of developing a disease, where pre-pathologic and/or pathologic cells belong to a signature predefined class of cells, the finding of 1 cell in this predefined class indicates that the in the case of a pre-pathological condition, the disease process has begun, or, in the case of a pathological condition, the individual is already afflicted, but may be yet to manifest disease symptoms. Even in otherwise healthy individuals, the appearance of a single cell of a particular state indicates that pathology or disease is present. For example, the appearance in a blood sample of a single cell in a predefined class known to be that of a certain category of cancer indicates the presence of such a cancer, whether or not other findings indicate any disease presence. Such a finding would allow early treatment, that may be less toxic and/or be associated with a greater degree of disease control or cure. In some embodiments, the appearance of one cell in two or more different predefined classes indicates a particular disease status. In some embodiments, the minimal status of a pathological state is determined by a finding of even a single cell.

In some cases, the number of cells in a predefined class may be determinative of an individual's status only if the number exceeds or is less than a certain threshold number of cells. For example, a threshold number may represent a clinically observed dividing line, associated with patient outcome. Individuals above the threshold may have a worse prognosis than those below the threshold number and may require more immediate and/or more aggressive treatment than individuals below the threshold number. The threshold number can be theoretically, or, more typically, empirically derived, e.g., from retrospective analysis of clinical samples as described herein. In some instances, the threshold number is 0 and the finding of cells in the predefined class would indicate that the status of the individual has changed and treatment must begin. In other instances, the threshold number is 1. In still other instances, the threshold number is 2, 3, 4, 5, 6, 7, 8, 9, 10, 15, 20, 30, 40, 50, 60, 70, 80, 90, 100, 150, 200, 300, 400, 500, 600, 700, 800, 900, 1000, 5,000, 10,000, 100,000, or 1,000,000 cells.

In some embodiments, the number of cells that will be determinative of the individual status will depend on the phenotype of the cells in the predefined class. For example, in determining the probability of relapse in cancer patients, patients that have cells associated with a malignant phenotype would have relapses if they have number of cells in the predefined class higher than for example 10⁻⁵, whereas patients with cells associated with a less malignant phenotype would have relapses if they have number of cells in a predefined class higher than for example 10⁻².

In other embodiments, rather than a threshold number, the finding of a certain number of cells in a particular class in a sample from an individual may be correlated with a certain probability of a particular status for the individual. For example the presence of about 2, 3, 4, 5, 6, 7, 8, 9, 10, 15, 20, 30, 40, 50, 60, 70, 80, 90, 100, 150, 200, 300, 400, 500, 600, 700, 800, 900, 1000, 5,000, 10,000, 100,000, or 1,000,000 cells in a predefined class may be indicative of an individual's status. Ranges of cell numbers for a given condition of sampling (e.g., number of cells per 5 or 10 ml blood draw) are useful. Ranges may be any useful range that has been correlated to a particular outcome or status, and may be a minimum of about 1, 2, 3, 4, 5, 6, 7, 8, 9, 10, 15, 20, 30, 40, 50, 60, 70, 80, 90, 100, 150, 200, 300, 400, 500, 600, 700, 800, 900, 1000, 5,000, 10,000, 100,000, or 1,000,000 cells and a maximum of about 1, 2, 3, 4, 5, 6, 7, 8, 9, 10, 15, 20, 30, 40, 50, 60, 70, 80, 90, 100, 150, 200, 300, 400, 500, 600, 700, 800, 900, 1000, 5,000, 10,000, 100,000, 1,000,000 or 10,000,000 cells. For example, for a blood draw under certain defined conditions (e.g., a blood draw of a particular volume, or normalized to a particular volume) which contains a certain number of cells in a predefined class, may indicate that an individual is at a certain percentage of risk for developing a certain condition within a given time. As an example only, the presence of 10-100 cells of a certain predefined class in a blood draw of 10 ml may be associated with a 50% probability of pathology occurring within 5 years. It will be appreciated that ranges and probabilities may be adjusted as databases become more extensive.

In some embodiments, the number of cells in a predefined class may be determinative when the number of cells is fewer than 10⁻³ to 10⁻⁴ cells (one cell in the predefined class in 1,000 to 10,000 total cells). For example, the presence of 1×10⁻³, 2×10⁻³, 3×10⁻³, 4×10⁻³, 5×10⁻³, 6×10⁻³, 7×10⁻³, 8×10⁻³, 9×10⁻³, 1×10⁻⁴, 2×10⁻⁴, 3×10⁻⁴, 4×10⁻⁴, 5×10⁻⁴, 6×10⁻⁴, 7×10⁴, 8×10⁻⁴, or 9×10⁻⁴ cells in a predefined class may be indicative of an individual's status. In some embodiments, the number of cells in a predefined class maybe determinative when the number of cells is fewer than 5×10⁻⁴ In some embodiments, the number of cells in a predefined class maybe determinative when the number of cells is fewer than 4.5×10⁻⁴. In some embodiments, the number of cells in a predefined class maybe determinative when the number of cells is fewer than 4×10⁻⁴. In some embodiments, the number of cells in a predefined class maybe determinative when the number of cells is fewer than 3.5×10⁻⁴. In some embodiments, the number of cells in a predefined class maybe determinative when the number of cells is fewer than 3.5×10⁻⁴. In some embodiments, the number of cells in a predefined class maybe determinative when the number of cells is fewer than 2×10⁻⁴.

In some embodiments, the number of cells in a predefined class may be determinative when the number of cells is higher than 10⁻² to 10⁻⁴ cells (one cell in the predefined class in 100 to 10,000 total cells). For example, the presence of 1×10⁻², 2×10⁻², 3×10⁻², 4×10⁻², 5×10⁻², 6×10⁻², 7×10⁻², 8×10⁻², 9×10⁻², 1×10⁻³, 2×10⁻³, 3×10⁻³, 4×10⁻³, 5×10⁻³, 6×10⁻³, 7×10⁻³, 8×10⁻³, 9×10⁻³ or 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ cells in a predefined class may be indicative of an individual's status. In some embodiments, the number of cells in a predefined class maybe determinative when the number of cells is higher than 5×10⁻⁴. In some embodiments, the number of cells in a redefined class maybe determinative when the number of cells is higher than 4.5×10⁻⁴. In some embodiments, the number of cells in a predefined class maybe determinative when the number of cells is higher than 4×10⁻⁴. In some embodiments, the number of cells in a predefined class maybe determinative when the number of cells is higher than 3.5×10⁻⁴. In some embodiments, the number of cells in a predefined class maybe determinative when the number of cells is higher than 3.5×10⁻⁴. In some embodiments, the number of cells in a predefined class maybe determinative when the number of cells is higher than 2×10⁻⁴.

In some embodiments, the number of cells in a predefined class may be determinative when it is correlated with a predetermined clinical parameter. For example in determining the probability of relapse in AML patients, patients that have a favorable cytogenetic subtype would have relapses if they have number of cells in a predefined class higher than for example 10⁻², whereas patients with adverse cytogenetic subtypes would have relapses if they have number of cells in a predefined class higher than for example 10⁻⁴. In other diseases, the presence of even one cell in a predefined class may indicate a relapse.

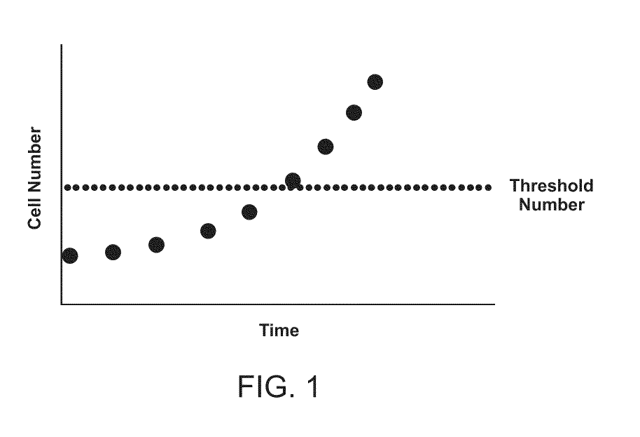

When a series of samples is taken over time, a predefined class of cells can be analyzed to see if it is increasing or decreasing in number at a rate that will cause the predefined class of cells to cross a threshold number in the future. FIG. 1 illustrates this situation; in this case, a series of samples is analyzed and at a certain point the number of cells in a particular class crosses a threshold indicating a change in status. By predicting when an individual may cross a threshold number, earlier action may be taken to either prevent the crossing of the threshold number in cases where crossing is associated with a detrimental outcome, or accelerate the crossing of the threshold number where crossing is associated with a better outcome or prognosis. For example, if the trend shows that a particular predefined class of cells in patient associated with the relapse of disease will cross the threshold number in a month, prophylactic treatment can be initiated to prevent the occurrence.

In some cases, the rate of change of the number of cells in a predefined class may be an indicator of present or future health status. This may be combined with absolute numbers, or used as a further indicator with an absolute number. This is similar to the situation with PSA, where a low absolute number is generally taken as a sign of healthy prostate, but if an increase is seen over a series of samples further testing is indicated, even if each individual number is in itself not indicative of pathology. As with threshold values or ranges, certain rates of change, or ranges of rates of change, may be associated with certain probabilities of outcome; such probabilities may be modified based on the absolute number of cells in the predefined class; e.g., a low rate of change couple with high absolute numbers may indicate the same probability of a given outcome or health status as a high rate of change coupled with low absolute numbers.

In some embodiments of the invention, a series of samples is taken from an individual undergoing treatment for a condition, e.g., treatment for a cancer. The samples may be evaluated for the number of cells that correlate with the cancer, and the rate of change in numbers of these cells during treatment may be correlated with a particular outcome; e.g., a rapid decrease in cancer cell number may indicate a more positive prognosis than a less rapid decrease; in addition, changes in the rate of change (e.g., rapid decrease followed by little or no decrease) also may have prognostic value. Such evaluations of the rate of change during treatment may be combined with numbers of cells in one or more predefined classes at the conclusion of treatment, and/or after treatment, to further refine the prognostic and diagnostic accuracy.

The threshold number for a particular predefined class may differ based on sample location. For example, cells isolated from peripheral blood and those from bone marrow or lymph nodes may have their own clinically significant threshold numbers for specific predefined classes of cells. Ratios and other mathematical methods of comparison may be developed to allow the comparison of cells isolated from different bodily locations, thereby providing greater flexibility to the clinician in procuring a sample of a plurality of cells.

When more than one predefined class of cells are present, comparative quantitative analyses can be performed to determine an individual's status (e.g., health status). Numerous comparative and statistical techniques are known in the arts for the analysis of different groups. Examples of such statistical methods include but are not limited to X²-test, Student T test, Mann-Whitney U test, log-rank, Breslow test, Kaplan, Meier, Spearman's rank correlation, logistic regression model, Cox models, or AUC plots. In some embodiments, the p value in the analysis is below 0.05, 04, 0.03, 0.02, 0.01, 0.009, 0.005, or 0.001. In some embodiments, the p value is below 0.001. Thus in some embodiments, the status of an individual can be determined by performing a quantitative analysis on one or more predefined classes of cells wherein the p value is below 0.05, 04, 0.03, 0.02, 0.01, 0.009, 0.005, or 0.001. In some embodiments, the p value is below 0.001. In some embodiments, the status of an individual can be determined by performing a quantitative analysis on one or more predefined classes of cells wherein the AUC value is higher than 0.5, 0.6, 07, 0.8 or 0.9. In some embodiments, the status of an individual can be determined by performing a quantitative analysis on one or more predefined classes of cells wherein the AUC value is higher than 0.7. In some embodiments, the status of an individual can be determined by performing a quantitative analysis on one or more predefined classes of cells wherein the AUC value is higher than 0.8. In some embodiments, the status of an individual can be determined by performing a quantitative analysis on one or more predefined classes of cells wherein the AUC value is higher than 0.9.

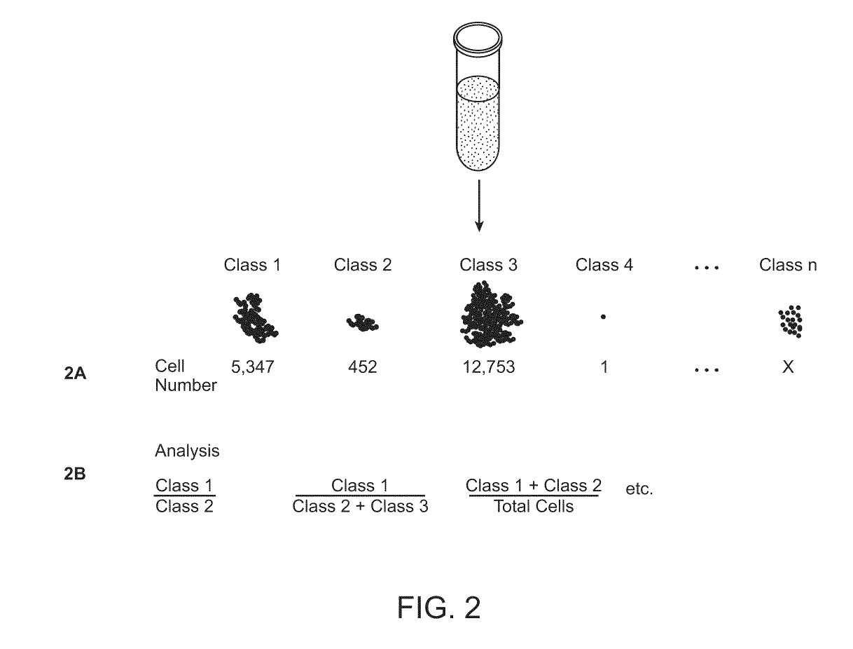

In some embodiments, the number of cells in one predefined class can be compared to the number of cells in another predefined class by taking the ratio of the two. FIG. 2 illustrates a situation in which cells are quantitated in a number of different predefined classes; FIG. 2B shows various exemplary ratios that could be obtained. Alternately, the number of cells in one predefined class can be compared by taking the ratio of this class and the cell number from a combination of predefined classes. As a simple example, if predefined class 1 has 200 cells and predefined class 2 has 1000 cells, the ratio of cells in A to cells in B would be 0.2, or 20%.

The simplest ratio is the ratio of one predefined class of cells to total cells. In this case, the term “total cells” includes all cells in the sample, total cells collected for analysis or total live cells analyzed, whether or not their status, e.g., the activation level of their intracellular activatable elements, has been determined. Thus, “total cells” includes a predefined class that encompasses the total of any cell in the sample. In some embodiments, the ratio is that of one predefined class to total cells of a certain type, e.g., total leukocytes, or total cells with a particular set of cell surface markers.

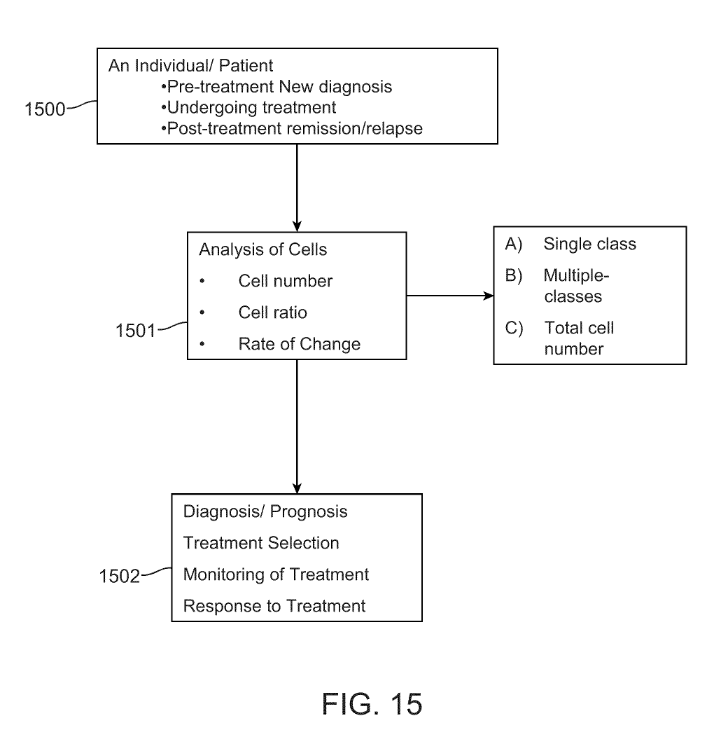

FIG. 15 is a diagram showing the method of determining a status of an individual (e.g. health status) at a certain stage, In some embodiments, the method of the present invention can be applied to an individual before a diagnosis, an individual undergoing a treatment, or an individual in remission or having a relapse as depicted in step 1500 of FIG. 15. In step 1501, cells from the individual are analyzed according to the method described herein. In some embodiments, one or more samples are taken from the individual, and subjected to a modulator, as described herein. In some embodiments, the sample is divided into subsamples that are each subjected to a different modulator. After treatment with the modulator, single cells in the sample or subsample are analyzed to determine the activation level of one or more activatable elements. Any suitable form of analysis that allows a determination of cell activation level(s) may be used. In some embodiments, the analysis includes the determination of the activation level of an intracellular element, e.g., a protein. In some embodiments, the analysis includes the determination of the activation level of an activatable element, e.g., an intracellular activatable element such as a protein, e.g., a phosphoprotein. The analysis of activation level of an intracellular element, e.g., a protein, may be achieved by the use of activation state-specific binding elements, such as antibodies, as described herein. A plurality of activatable elements may be examined.

In step 1501 of FIG. 15, cells are analyzed by determining the number of cells, cell ratio or rate of change. The analysis can be performed by any method described herein such as the method described in FIGS. 1 to 4. In some embodiments, the p value is below 0.05, 04, 0.03, 0.02, 0.01, 0.009, 0.005, or 0.001. In some embodiments, the p value is below 0.001. In some embodiments, the AUC value is higher than 0.5, 0.6, 07, 0.8 or 0.9. In some embodiments, the AUC value is higher than 0.8.

In step 1502 of FIG. 15, a diagnosis, prognosis, method of treatment or response to treatment is determined after the analysis in step 1501. Thus the analysis of step 1501 allows for the diagnosis, prognosis, choice or modification of treatment, and/or monitoring of a disease, disorder, or condition. In some embodiments, the determination of the status of an individual can be determining whether the individual is in the normal range for a particular condition or whether the individual has a pre-pathological or pathological condition warranting monitoring and/or treatment. In some embodiments, the determination of the status of an individual can be determining the minimal status of a pathological state. The determination of the status may also indicate response of an individual to treatment for a condition. In some embodiments, the determination of the status of an individual may be used to ascertain whether a previous condition or treatment has induced a new pre-pathological or pathological condition that requires monitoring and/or treatment. In another embodiment, the status of an individual can indicate an individual's predicted or actual response to treatment for a pre-pathological or pathological condition. In some embodiments, the analysis obtained in step 1501 may be used to determine the best therapy for an individual, which may include the determination that the best therapy for a patient is supportive care. In a further embodiment, the status of an individual may indicate an individual's immunologic status and may reflect a general immunologic status, an organ or tissue specific status, or a disease related status.

It will be appreciated that further ratios are possible. The combinations are limited only by the number of classes present in the sample. It will also be appreciated that databases may be constructed for all such ratios, and that any such ratio that has a correlation with the status of an individual may be used in the methods of the invention.

Ratios may be used alone, or in combination with numbers of cells in single classes, to provide an indication of the status of the individual. Thus, all analyses described herein for threshold analysis, rate of change analysis, absolute number analysis, or combinations thereof, also apply to ratios of cells.

In some embodiments, a ratio of about 0, 0.0000001%, 0.000001%, 0.00001%, 0.0001%, 001%, 0.005%, 0.01%, 0.05%, 0.1%, 0.5%, 1.0%, 5.0%, 10%, 20%, 40%, 60%, 80%, 90%, 95%, or 100% can be determinative of an individual's status. In other embodiments, whether the calculated ratio lies above or below a threshold ratio is also determinative. The threshold ratio may be about 0, 0.0000001%, 0.000001%, 0.00001%, 0.0001%, 001%, 0.005%, 0.01%, 0.05%, 0.1%, 0.5%, 1.0%, 5.0%, 10%, 20%, 40%, 60%, 80%, 90%, 95%, or 100%. For example, in some embodiments, the existence of minimal residual disease after treatment may be when the ratio of the number of cells exhibiting a cancerous state to total cells in a sample, e.g., a blood sample, exceeds a certain percentage, such as 0.0001%, 0.001%, 0.01%, or 0.1%.

As with absolute numbers, it will often be useful to correlate a ratio of predefined classes with a probability of an outcome; in some embodiments, a range of ratios may be correlated with a probability. Such a range may be from a minimum of 0, 0.0000001%, 0.000001%, 0.00001%, 0.0001%, 001%, 0.005%, 0.01%, 0.05%, 0.1%, 0.5%, 1.0%, 5.0%, 10%, 20%, 40%, 60%, 80%, 90%, 95% to a maximum of 0.0000001%, 0.000001%, 0.00001%, 0.0001%, 001%, 0.005%, 0.01%, 0.05%, 0.1%, 0.5%, 1.0%, 5.0%, 10%, 20%, 40%, 60%, 80%, 90%, 95%, or 100%.

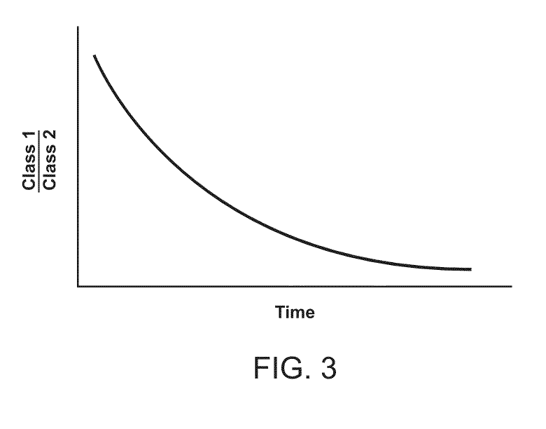

In some embodiments, where multiple samples are available sequentially over time from the same location, the ratio between particular predefined classes of cells can be analyzed to see if the ratio is trending in a particular direction, just as for absolute numbers. FIG. 3 illustrates such an analysis. If the ratio appears that it may cross a threshold ratio in the future, prophylactic treatment or other desirable course of action can be taken to prevent or accelerate the ratio from crossing the threshold ratio.

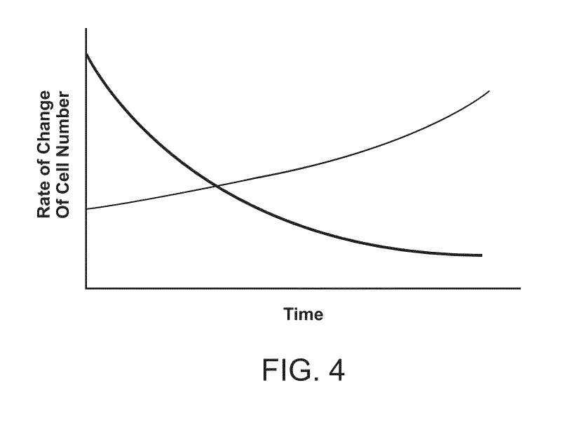

When sequential samples are available, a predefined class of cells can also be analyzed by measuring the rate of change in the cell number within the class (see FIG. 4). One common measurement of the rate of change is the doubling time of the number of cells in a predefined class. When data from multiple predefined classes is available over time, the rate of change in the ratio between the classes can also be measured.

In some embodiments, the rate of activation or deactivation of a particular intracellular activatable element with a specific modulator or class of modulators may define a predefined class. The activation rate/deactivation rate can be determined through sequential measurements on cells obtained at different time points from the same source or location. Alternatively, the activation/deactivation rate can be determined from a plurality of cells that are obtained at the same time, but are assayed over time.

While some embodiments are associated with placing single cells in predefined classes, in other embodiments, the appearance of one or more cells outside the predefined classes may be indicative of significant changes in the status of an individual. Of particular interest in determining the status of an individual is the detection and analysis of classes of cells that have one or more different activation levels compared to normal control values, or to previous determinations made from a sample from the individual. The different activation levels can be the result of the disappearance of one or more previous identified activation levels from one or more predefined classes of cells. Alternatively, the different activation levels may be the result of the appearance of a new, activation level. The analysis of cells with one or more different activation levels is the same as for other classes of cells, but cells that have deletions of or additions to previously identified activation levels are often of greater clinical significance. For example, a hallmark of cancer is genomic instability. The appearance of a class of cells with one or more different activation levels during the course of cancer treatment may signify that a mutation has occurred and a new clonal population has arisen. Mutations in such instances are frequently associated with increased resistance to the employed treatment agents and such cells often comprise a major portion of the cancerous cells when a patient experiences a recurrence.

In the determination of the status of an individual along a health continuum, other factors can be considered. Any factor that gives additional predictive or diagnostic power to the single cell analyses described herein may be used. Such factors are well-known in the art. These include an individual's gender; race; current age; age at the time of disease presentation; age at the time of treatment; clinical stage of disease; genetic results, number of previous therapies; type of previous therapies; response to previous therapy or therapies; time from last treatment; blood cell count; bone marrow reserves; and performance status, patient's past medical history, family history of any medical problems, patient's social history, as well as any current medical history termed “review of systems”, and physical exam findings. Other factors are more specific to the specific condition being evaluated, e.g., percentage of blasts in bone marrow as an indicator of certain leukemias. Such factors are well-known in the art for particular diseases and conditions. Examples of tests that can be performed together with the methods described herein include, but are not limited to, immunophenotyping, morphology, conventional cytogenetics, molecular cytogenetics, molecular genetics and HLA typing.

### Status of the Individual

The techniques and methods of this invention allow for the determination of the status of an individual for any condition for which a correlation between the condition, its prognosis, course of treatment, or other relevant characteristic, and the state of single cells, e.g., activation level of one or more activatable elements, in samples from individuals may be ascertained. In some embodiments, samples are blood samples and conditions that may be examined using the techniques of the invention are those that cause alterations in single cells found in blood samples. However, the invention is not limited to the use of blood samples, and any condition which leads to a change in single cells in an area of the individual amenable to sampling may be examined by the techniques of the invention.

In some embodiments, the invention provides a method of predicting a change in a health status in an individual from a first state to a second state comprising: determining the presence of a first and second class of cells in a sample from the individual, the presence being determined by a method comprising determining an activation level of an intracellular activatable element in single cells from said sample, classifying single cells into the first and second class, wherein at least one class is classified based on the activation level; calculating a ratio of the first and second class of cells and using the ratio to predict said change in health status; and predicting a change in a health status in the individual from a first state to a second state when said ratio exceeds a threshold number. In some embodiments, the threshold number expressed as a percentage is 30%. In some embodiments, the threshold number expressed as a percentage is 5%. In some embodiments threshold number expressed as a percentage is 1%. In some embodiments, the threshold number expressed as cell frequency is 10⁻². In some embodiments, the threshold number expressed as cell frequency is 10⁻³. In some embodiments, the threshold number expressed as cell frequency is 10⁻⁴.

In some embodiments, the health status or the predicted health status of an individual places the individual along a health continuum that typically runs from a normal or healthy state to one or more pre-pathologic states, and finally to a pathologic state. In some instances, the health continuum may run from a normal state to a pathological state without an intervening pre-pathologic state. The health continuum may also comprise a partial continuum of the aforementioned states or a portion of one state. The health continuum may be related to the general health status of an individual, an organ or organ system or the individual component tissues of an organ. Additionally, the health continuum may be specific for a family of related diseases or disorders, a particular disease or disorder or subtypes of a disease or disorder.

In some embodiments, an individual to be evaluated has not been diagnosed with a pre-pathologic or pathologic condition but is undergoing a screening. In some embodiments, the minimal status of a pathological state is determined. In certain instances, the finding of 0 cells associated with a pathological state may be determinative as to minimal status of a pathological state. For example, the finding of 0 cells associated with a pathological state provides evidence that the individual does not have the pathological state or has not experienced a recurrence. In some embodiments, the presence of 1 cell associated with a pathological state may be determinative of an individual's status. In this case, the threshold number is 0, and finding even a single cell (more than zero) is indicative of the minimal status of the pathological state. For example, the finding of 1 cell that is associated with a highly malignant cancer phenotype indicates that the in the case of cancer, the disease process has begun, but may be yet to manifest disease symptoms. In an individual who has been treated for the pathological condition, the detection of cells associated with the pathological state indicates that treatment is incomplete. In other instances, a finding of a number higher than a threshold of cells associated with a pathological state may be determinative of an individual's status. For example, a finding of equal or higher that 10⁻⁴ cells associated with a cancer phenotype may indicate that the individual is at risk of having a relapse, whereas a finding of less than 10⁻⁴ cells may indicate that the individual is at very low risk of relapse. The minimal status of a pathological state can thus distinguish who needs intensive and potentially more toxic therapy from those who do not. In some cases the minimal status may also inform on the timing of a clinical intervention.

In these embodiments, one or more samples may be taken from the individual, and subjected to a modulator, as described herein. In some embodiments, the sample is divided into subsamples that are each subjected to a different modulator. After treatment with the modulator, single cells in the sample or subsample are analyzed to determine their activation level(s). Any suitable form of analysis that allows a determination of cell activation level(s) may be used. In some embodiments, the analysis includes the determination of the activation level of an intracellular element, e.g., a protein. In some embodiments, the analysis includes the determination of the activation level of an activatable element, e.g., an intracellular activatable element such as a protein, e.g., a phosphoprotein. Determination of the status may be achieved by the use of activation state-specific binding elements, such as antibodies, as described herein. A plurality of activatable elements may be examined. Single cells may be placed into predefined classes, and the status of the individual determined based on the classes into which cells are categorized. In some embodiments, a quantitative analysis of the number of cells in one or more classes is performed to determine the status of the individual. In some embodiments, the status to be determined includes the emergence of a new pre-pathologic or pathologic condition, including a malignancy. Diagnosis, prognosis, and/or a course of treatment may also be determined based on the analysis of the classes of cells. In some embodiments, the p value in the analysis is below 0.05, 04, 0.03, 0.02, 0.01, 0.009, 0.005, or 0.001. In some embodiments, the p value is below 0.001. In some embodiments, the AUC value is higher than 0.5, 0.6, 07, 0.8 or 0.9. In some embodiments, the AUC value is higher than 0.8.

In some embodiments, an individual to be evaluated has already been subjected to at least one form of treatment for a condition, e.g., a malignancy. In some embodiments, the invention provides methods of the determination of the minimal residual status of disease in an individual who has received treatment. In these embodiments, one or more samples may be taken from the individual, and subjected to one or more modulators, as described herein. In some embodiments, the sample is divided into subsamples that are each subjected to one or more different modulators. After treatment with one or more modulators, single cells in the sample or subsample are analyzed to determine their activation level(s). Any suitable form of analysis that allows a determination of cell activation level(s) may be used. In some embodiments, the analysis includes the determination of the activation level of an intracellular element, e.g., a protein. In some embodiments, the analysis includes the determination of the activation level of an activatable element, e.g., an intracellular activatable element such as a protein, e.g., a phosphoprotein. Determination of the status may be achieved by the use of activation state-specific binding elements, such as antibodies, as described herein. A plurality of activatable elements may be examined. Single cells may be placed into predefined classes, and the status of the individual determined based on the classes into which cells are categorized. In some embodiments, a quantitative analysis of the number of cells in one or more classes is performed to determine the status of the individual. In some embodiments, the status to be determined includes no return of malignancy, return of malignancy, appearance of a new pathology, e.g., malignancy, which may be a result of treatment, or a combination (e.g., return of malignancy and appearance of a new pathology). Diagnosis, prognosis, and/or a course of treatment may also be determined based on the analysis of the classes of cells. See Haskell et al, Cancer Treatment, 5^{th} Ed., W.B. Saunders and Co., 2001.

In some embodiments, the invention provides a method of detecting the minimal residual status of disease in an individual who has received treatment comprising subjecting a plurality of cells in a sample from an individual to a modulator; determining the activation levels of a plurality of intracellular activatable elements in single cells in response to the modulator by a process comprising the binding of a plurality of binding elements which are specific to a particular activation state of a particular activatable element, wherein the single cells are placed into one or more classes based on said response to said modulator or modulators; determining the presence or absence of said disease-associated cells based on the response, wherein determining the presence or absence of the disease-associated cells comprises quantitative analysis of the one or more classes; and determining the minimal residual status of a disease, wherein the minimal residual status is based on the presence or absence of a small number of the disease-associated cells.

In some embodiments, diagnosis, prognosis and/or selection of treatment course of a disease comprises tracking the emergence and disappearance of rare cell populations.

In some embodiments, the determination of status is the presence of residual malignant cells, even when there are so few cancer cells present (e.g., even one cancer cell) that they cannot be found by routine diagnostic modalities. The detection of residual malignant cells indicates that treatment is incomplete. The methods of the invention can thus distinguish between individuals who need additional intensive and potentially more toxic therapy from those individuals who do not.

In some embodiments, the determination of status comprises the presence and characteristics of cancer stem cells, which are a very low minority of the whole tumor cells. Cancer stem cells frequently respond differently to therapeutic agents than do other tumor cells. Understanding these differences may be important in increasing the cure rates for cancer. Cancer stem cell characteristics that may be determined include chemotherapy or biological therapy target expression and response to therapy.

In some embodiments, the determination of status comprises the detection and functional characterization of immune cells specifically related to the pathogenesis of autoimmune diseases. Specific immune cells can be monitored over time while they are under therapeutic pressure either in vitro or in vivo to provide information to guide patient management.

Numerous immunologic, proliferative and malignant diseases and disorders are especially amenable to the methods described herein. Immunologic diseases and disorders include allergic diseases and disorders, disorders of immune function, and autoimmune diseases and conditions. Allergic diseases and disorders include but are not limited to allergic rhinitis, allergic conjunctivitis, allergic asthma, atopic eczema, atopic dermatitis, and food allergy. Immunodeficiencies include but are not limited to severe combined immunodeficiency (SCID), hypereosinophilic syndrome, chronic granulomatous disease, leukocyte adhesion deficiency I and II, hyper IgE syndrome, Chediak Higashi, neutrophilias, neutropenias, aplasias, Agammaglobulinemia, hyper-IgM syndromes, DiGeorge/Velocardial-facial syndromes and Interferon gamma-TH1 pathway defects. Autoimmune and immune dysregulation disorders include but are not limited to rheumatoid arthritis, diabetes, systemic lupus erythematosus, Graves' disease, Graves opthalmopathy, Crohn's disease, multiple sclerosis, psoriasis, systemic sclerosis, goiter and struma lymphomatosa (Hashimoto's thyroiditis, lymphadenoid goiter), alopecia aerata, autoimmune myocarditis, lichen sclerosis, autoimmune uveitis, Addison's disease, atrophic gastritis, myasthenia gravis, idiopathic thrombocytopenic purpura, hemolytic anemia, primary biliary cirrhosis, Wegener's granulomatosis, polyarteritis nodosa, and inflammatory bowel disease, allograft rejection and tissue destructive from allergic reactions to infectious microorganisms or to environmental antigens.

Proliferative diseases and disorders that may be evaluated by the methods of the invention include, but are not limited to, hemangiomatosis in newborns; secondary progressive multiple sclerosis; chronic progressive myelodegenerative disease; neurofibromatosis; ganglioneuromatosis; keloid formation; Paget's Disease of the bone; fibrocystic disease (e.g., of the breast or uterus); sarcoidosis; Peronies and Duputren's fibrosis, cirrhosis, atherosclerosis and vascular restenosis.

Malignant diseases and disorders that may be evaluated by the methods of the invention include both hematologic malignancies and solid tumors.

Hematologic malignancies are especially amenable to the methods of the invention when the sample is a blood sample, because such malignancies involve changes in blood-borne cells. Such malignancies include non-Hodgkin's lymphoma, Hodgkin's lymphoma, non-B cell lymphomas, and other lymphomas, acute or chronic leukemias, polycythemias, thrombocythemias, multiple myeloma, myelodysplastic disorders, myeloproliferative disorders, myelofibroses, atypical immune lymphoproliferations and plasma cell disorders.

Plasma cell disorders that may be evaluated by the methods of the invention include multiple myeloma, amyloidosis and Waldenstrom's macroglobulinemia.

Leukemias that may be evaluated by the invention include both myeloid and lymphoid leukemias. Myeloid leukemias include AML, CML, and juvenile myelomonocytic leukemia (JMML). Lymphoid leukemias include non-B cell acute lymphocytic leukemia (T-ALL), and B cell acute lymphoblastic leukemia (including pre-B cell) and chronic lymphocytic leukemia (CLL). Other hematologic diseases and disorders that may be evaluated by the methods of this invention include myeloid disorders such as myelodysplastic disorders, myeloproliferative disorders, myelofibroses, polycythemias, and thrombocythemias and others such as B cell immunoproliferations (post transplant lymphoproliferation disorder (PTLD) and non-B atypical immune lymphoproliferations. See Haskell et al, Cancer Treatment, 5^{th} Ed., W.B. Saunders and Co., 2001.

In some embodiments of the invention, the hematologic disease that is evaluated by the methods of the invention is CLL. Thus, in some embodiments the invention provides tracking of the disease course including the emergence and disappearance of rare cell populations, allowing for methods for diagnosing CLL, determining a method of treatment for CLL, determining a prognosis for CLL, or determining response to treatment for CLL in an individual, using the methods described herein. In some embodiments, the individual has been previously diagnosed with CLL and is undergoing or has undergone treatment for CLL. One or more blood samples are taken from the individual; in some embodiments a series of blood samples are taken from the individual over time. The samples may be taken before, during, or after treatment, or some combination thereof. In some embodiments, the samples are taken before, during, and after treatment. Additional samples or other diagnostic markers, as are known in the art, may also be used in addition to the blood samples to determine the status of the individual; e.g., bone marrow samples may be taken, and/or blood cells may examined for well-established markers of CLL, such as surface antigen markers, e.g., coexpression of CD5 with CD19 and CD23 or CD5 with CD20 and CD23 and dim surface immunoglobulin expression. In some embodiments, the samples or portions of the samples are treated with a modulator, and the state of single cells is determined, from which a determination is made as to the status of the CLL in the individual. In some embodiments, the state of single cells is the activation level of one or more activatable elements, e.g., proteins such as phosphoproteins, in the cells. Quantitative analysis, as described herein, is performed, in order to determine the status of the CLL in the individual. In some embodiments, a treatment decision is made based at least in part on the determination of the status of CLL using the methods described herein; such treatment decision may include no treatment, treatment with a previously-used treatment, modification of treatment, or use of a new treatment.

In some embodiments, the number of cells associated with CLL may be determinative when the number of cells is fewer than 10⁻³ to 10⁻⁴ cells. For example, the presence of 1×10⁻³, 2×10⁻³, 3×10⁻³, 4×10⁻³, 5×10⁻³, 6×10⁻³, 7×10⁻³, 8×10⁻³, 9×10⁻³, 1×10⁻⁴, 2×10⁻⁴, 3×10⁻⁴, 4×10⁻⁴, 5×10⁴, 6×10⁻⁴, 7×10⁴, 8×10⁻⁴, or 9×10⁻⁴ cells associated with CLL may be indicative of an individual's status. In some embodiments, the number of cells associated with CLL may be determinative when the number of cells is higher than 10⁻² to 10⁻⁴ cells. For example, the presence of 1×10⁻², 2×10⁻², 3×10⁻², 4×10⁻², 5×10⁻², 6×10⁻², 7×10⁻², 8×10⁻², 9×10⁻², 1×10⁻³, 2×10⁻³, 3×10⁻³, 4×10⁻³, 5×10⁻³, 6×10⁻³, 7×10⁻³, 8×10⁻³, 9×10⁻³ or 1×10⁻⁴, 2×10⁻⁴, 3×10⁻⁴, 4×10⁻⁴, 5×10⁴, 6×10⁻⁴, 7×10⁴, 8×10⁻⁴, or 9×10⁻⁴ cells associated with CLL may be indicative of an individual's status. In some embodiments, the number of cells associated with CLL may be determinative when it is correlated with a predetermined clinical parameter. For example in determining the probability of relapse in CLL patients, patients with specific cell surface proteins or older than certain age would have relapses if they have number of cells associated with CLL higher than for example 10⁻², whereas patients with different cell surface proteins or younger than certain age would have relapses if they have number of cells associated with CLL higher than for example 10⁻⁴.

In some embodiments of the invention, the hematologic disease that is evaluated by the methods of the invention is AML. Thus, in some embodiments, the invention provides methods for diagnosing AML, determining a method of treatment for AML, determining a prognosis for AML, or determining response to treatment for AML in an individual, using the methods described herein. In some embodiments, the individual has been previously diagnosed with AML and is undergoing or has undergone treatment for AML. One or more blood samples are taken from the individual; in some embodiments a series of blood samples are taken from the individual over time. The samples may be taken before, during, or after treatment, or some combination thereof. In some embodiments, the samples are taken before, during, and after treatment. Additional samples or other diagnostic markers, as are known in the art, may also be used in addition to the blood samples to determine the status of the individual; e.g., bone marrow samples may be taken, and/or blood cells may examined for well-established markers of AML including, but are not limited to, fetal liver tyrosine kinase/internal tandem duplication (FLT3/ITD), NPM1, ERG, KIT, thymidine-kinase expression levels, β2-microglobulin expression, the presence of MDR1 phenotype, or cytogenetic analysis to examine for the presence of abnormal karyotypes. In some embodiments diagnosis, prognosis, or method of treatment further relies on medical history and physical examination including, but not limited to past bone marrow or peripheral blood stem cell transplantation; total body irradiation with concurrent bone marrow or stem cell transplantation or any combination thereof. In some embodiments, the samples or portions of the samples are treated with a modulator, and the activation level of single cells is determined, from which a determination is made as to the status of the AML in the individual. In some embodiments, the activation level of single cells is the activation level of one or more activatable elements, e.g., proteins such as phosphoproteins, in the cells. Quantitative analysis, as described herein, is performed, in order to determine the status of the AML in the individual. In some embodiments, a treatment decision is made based at least in part on the determination of the status of AML using the methods described herein; such treatment decision may include no treatment, treatment with a previously-used treatment, modification of treatment, or use of a new treatment.

In some embodiments, the number of cells associated with AML may be determinative when the number of cells is fewer than 10⁻³ to 10⁻⁴ cells. For example, the presence of 1×10⁻³, 2×10⁻³, 3×10⁻³, 4×10⁻³, 5×10⁻³, 6×10⁻³, 7×10⁻³, 8×10⁻³, 9×10⁻³, 1×10⁻⁴, 2×10⁻⁴, 3×10⁻⁴, 4×10⁻⁴, 5×10⁴, 6×10⁻⁴, 7×10⁴, 8×10⁻⁴, or 9×10⁻⁴ cells associated with AML may be indicative of an individual's status. In some embodiments, the number of cells associated with AML may be determinative when the number of cells is higher than 10⁻² to 10⁻⁴ cells. For example, the presence of 1×10⁻², 2×10⁻², 3×10⁻², 4×10⁻², 5×10⁻², 6×10⁻², 7×10⁻², 8×10⁻², 9×10⁻², 1×10⁻³, 2×10⁻³, 3×10⁻³, 4×10⁻³, 5×10⁻³, 6×10⁻³, 7×10⁻³, 8×10⁻³, 9×10⁻³ or 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ cells associated with AML may be indicative of an individual's status. In some embodiments, the number of cells associated with AML may be determinative when it is correlated with a predetermined clinical parameter. For example in determining the probability of relapse in AML patients, patients that have a favorable cytogenetic subtype would have relapses if they have number of cells associated with AML higher than for example 10⁻², whereas patients with adverse cytogenetic subtypes (e.g. (15;17) PML-RARA, t(8;21) AML1-RUNX1T1 (AML-ETO), inv(16)) would have relapses if they have number of cells associated with AML higher than for example 10⁻⁴.

In some embodiments of the invention, the hematologic disease that is evaluated by the methods of the invention is ALL. Thus, in some embodiments the invention provides methods for diagnosing ALL, determining a method of treatment for ALL, determining a prognosis for ALL, or determining response to treatment for ALL in an individual, using the methods described herein. In some embodiments, the individual has been previously diagnosed with ALL and is undergoing or has undergone treatment for ALL. One or more blood samples are taken from the individual; in some embodiments a series of blood samples are taken from the individual over time. The samples may be taken before, during, or after treatment, or some combination thereof. In some embodiments, the samples are taken before, during, and after treatment. Additional samples or other diagnostic markers, as are known in the art, may also be used in addition to the blood samples to determine the status of the individual; e.g., bone marrow samples may be taken, and/or blood cells may examined for well-established markers of ALL. In some embodiments, the samples or portions of the samples are treated with a modulator, and the activation level of single cells is determined, from which a determination is made as to the status of the ALL in the individual. In some embodiments, the activation level of single cells is the activation level of one or more activatable elements, e.g., proteins such as phosphoproteins, in the cells. Quantitative analysis, as described herein, is performed, in order to determine the status of the ALL in the individual. In some embodiments, a treatment decision is made based at least in part on the determination of the status of ALL using the methods described herein; such treatment decision may include no treatment, treatment with a previously-used treatment, modification of treatment, or use of a new treatment.

In some embodiments, the number of cells associated with ALL may be determinative when the number of cells is fewer than 10⁻³ to 10⁻⁴ cells. For example, the presence of 1×10⁻³, 2×10⁻³, 3×10⁻³, 4×10⁻³, 5×10⁻³, 6×10⁻³, 7×10⁻³, 8×10⁻³, 9×10⁻³, 1×10⁻⁴, 2×10⁻⁴, 3×10⁻⁴, 4×10⁻⁴, 5×10⁴, 6×10⁻⁴, 7×10⁻⁴, 8×10⁻⁴, or 9×10⁻⁴ cells associated with ALL may be indicative of an individual's status. In some embodiments, the number of cells associated with ALL may be determinative when the number of cells is higher than 10⁻² to 10⁻⁴ cells. For example, the presence of 1×10⁻², 2×10⁻², 3×10⁻², 4×10⁻², 5×10⁻², 6×10⁻², 7×10⁻², 8×10⁻², 9×10⁻², 1×10⁻³, 2×10⁻³, 3×10⁻³, 4×10⁻³, 5×10⁻³, 6×10⁻³, 7×10⁻³, 8×10⁻³, 9×10⁻³ or 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ cells associated with ALL may be indicative of an individual's status. In some embodiments, the number of cells associated with ALL may be determinative when it is correlated with a predetermined clinical parameter. For example in determining the probability of relapse in ALL patients, patients that have a favorable cytogenetic subtype would have relapses if they have number of cells associated with ALL higher than for example 10⁻², whereas patients with adverse cytogenetic subtype (e.g., t(9;22) BCR-ABL, t(12;21) ETV6-RUNX1 (TEL-AML1)) would have relapses if they have number of cells associated with ALL higher than for example 10⁻⁴.

In some embodiments of the invention, the hematologic disease that is evaluated by the methods of the invention is CML. Thus, in some embodiments the invention provides methods for diagnosing CML, determining a method of treatment for CML, determining a prognosis for CML, or determining response to treatment for CML in an individual, using the methods described herein. In some embodiments, the individual has been previously diagnosed with CML and is undergoing or has undergone treatment for CML. One or more blood samples are taken from the individual; in some embodiments a series of blood samples are taken from the individual over time. The samples may be taken before, during, or after treatment, or some combination thereof. In some embodiments, the samples are taken before, during, and after treatment. Additional samples or other diagnostic markers, as are known in the art, may also be used in addition to the blood samples to determine the status of the individual; e.g., bone marrow samples may be taken, and/or blood cells may examined for well-established markers of CML. In some embodiments, the samples or portions of the samples are treated with a modulator, and the state of single cells is determined, from which a determination is made as to the status of the CML in the individual. In some embodiments, the state of single cells is the activation level of one or more activatable elements, e.g., proteins such as phosphoproteins, in the cells. Quantitative analysis, as described herein, is performed, in order to determine the status of the CML in the individual. In some embodiments, a treatment decision is made based at least in part on the determination of the status of CML using the methods described herein; such treatment decision may include no treatment, treatment with a previously-used treatment, modification of treatment, or use of a new treatment.

In some embodiments, the number of cells associated with CML may be determinative when the number of cells is fewer than 10⁻³ to 10⁻⁴ cells. For example, the presence of 1×10⁻³, 2×10⁻³, 3×10⁻³, 4×10⁻³, 5×10⁻³, 6×10⁻³, 7×10⁻³, 8×10⁻³, 9×10⁻³, 1×10⁻⁴, 2×10⁻⁴, 3×10⁻⁴, 4×10⁻⁴, 5×10⁻⁴, 6×10⁻⁴, 7×10⁴, 8×10⁻⁴, or 9×10⁻⁴ cells associated with CML may be indicative of an individual's status. In some embodiments, the number of cells associated with CML may be determinative when the number of cells is higher than 10⁻² to 10⁻⁴ cells. For example, the presence of 1×10⁻², 2×10⁻², 3×10⁻², 4×10⁻², 5×10⁻², 6×10⁻², 7×10⁻², 8×10⁻², 9×10⁻², 1×10⁻³, 2×10⁻³, 3×10⁻³, 4×10⁻³, 5×10⁻³, 6×10⁻³, 7×10⁻³, 8×10⁻³, 9×10⁻³ or 1×10⁻⁴, 2×10⁻⁴, 3×10⁻⁴, 4×10⁻⁴, 5×10⁴, 6×10⁻⁴, 7×10⁻⁴, 8×10⁻⁴, or 9×10⁻⁴ cells associated with CML may be indicative of an individual's status. In some embodiments, the number of cells associated with CML may be determinative when it is correlated with a predetermined clinical parameter. For example in determining the probability of relapse in CML patients, patients that have a favorable cytogenetic subtype would have relapses if they have number of cells associated with CML higher than for example 10⁻², whereas patients with adverse cytogenetic subtype (e.g., t(9;22) BCR-ABL) would have relapses if they have number of cells associated with CML higher than for example 10⁻⁴.

In some embodiments of the invention, the hematologic disease that is evaluated by the methods of the invention is follicular lymphoma. Thus, in some embodiments the invention provides methods for diagnosing follicular lymphoma, determining a method of treatment for follicular lymphoma, determining a prognosis for follicular lymphoma, or determining response to treatment for follicular lymphoma in an individual, using the methods described herein. In some embodiments, the individual has been previously diagnosed with follicular lymphoma and is undergoing or has undergone treatment for follicular lymphoma. One or more blood samples are taken from the individual; in some embodiments a series of blood samples are taken from the individual over time. The samples may be taken before, during, or after treatment, or some combination thereof. In some embodiments, the samples are taken before, during, and after treatment. Additional samples or other diagnostic markers, as are known in the art, may also be used in addition to the blood samples to determine the status of the individual; e.g., bone marrow samples may be taken, and/or blood cells may examined for well-established markers of follicular lymphoma. In some embodiments, the samples or portions of the samples are treated with a modulator, and the state of single cells is determined, from which a determination is made as to the status of the follicular lymphoma in the individual. In some embodiments, the activation level of single cells is the activation level of one or more activatable elements, e.g., proteins such as phosphoproteins, in the cells. Quantitative analysis, as described herein, is performed, in order to determine the status of the follicular lymphoma in the individual. In some embodiments, a treatment decision is made based at least in part on the determination of the status of follicular lymphoma using the methods described herein; such treatment decision may include no treatment, treatment with a previously-used treatment, modification of treatment, or use of a new treatment.

In some embodiments, the number of cells associated with follicular lymphoma may be determinative when the number of cells is fewer than 10⁻³ to 10⁻⁴ cells. For example, the presence of 1×10⁻³, 2×10⁻³, 3×10⁻³, 4×10⁻³, 5×10⁻³, 6×10⁻³, 7×10⁻³, 8×10⁻³, 9×10⁻³, 1×10⁻⁴, 2×10⁻⁴, 3×10⁻⁴, 4×10⁻⁴, 5×10⁴, 6×10⁻⁴, 7×10⁻⁴, 8×10⁻⁴, or 9×10⁻⁴ cells associated with follicular lymphoma may be indicative of an individual's status. In some embodiments, the number of cells associated with follicular lymphoma may be determinative when the number of cells is higher than 10⁻² to 10⁻⁴ cells. For example, the presence of 1×10⁻², 2×10⁻², 3×10⁻², 4×10⁻², 5×10⁻², 6×10⁻², 7×10⁻², 8×10⁻², 9×10⁻², 1×10⁻³, 2×10⁻³, 3×10⁻³, 4×10⁻³, 5×10⁻³, 6×10⁻³, 7×10⁻³, 8×10⁻³, 9×10⁻³ or 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ cells associated with follicular lymphoma may be indicative of an individual's status. In some embodiments, the number of cells associated with follicular lymphoma may be determinative when it is correlated with a predetermined clinical parameter. For example in determining the probability of relapse in follicular lymphoma patients, patients that have a favorable cytogenetic subtype would have relapses if they have number of cells associated with follicular lymphoma higher than for example 10⁻², whereas patients with adverse cytogenetic subtype (e.g., t(14;18) IgH/BCL2) would have relapses if they have number of cells associated with follicular lymphoma higher than for example 10⁻⁴.

In some embodiments of the invention, the hematologic disease that is evaluated by the methods of the invention is mantle cell lymphoma. Thus, in some embodiments the invention provides methods for diagnosing mantle cell lymphoma, determining a method of treatment for mantle cell lymphoma, determining a prognosis for mantle cell lymphoma, or determining response to treatment for mantle cell lymphoma in an individual, using the methods described herein. In some embodiments, the individual has been previously diagnosed with mantle cell lymphoma and is undergoing or has undergone treatment for mantle cell lymphoma. One or more blood samples are taken from the individual; in some embodiments a series of blood samples are taken from the individual over time. The samples may be taken before, during, or after treatment, or some combination thereof. In some embodiments, the samples are taken before, during, and after treatment. Additional samples or other diagnostic markers, as are known in the art, may also be used in addition to the blood samples to determine the status of the individual; e.g., bone marrow samples may be taken, and/or blood cells may examined for well-established markers of mantle cell lymphoma. In some embodiments, the samples or portions of the samples are treated with a modulator, and the state of single cells is determined, from which a determination is made as to the status of the mantle cell lymphoma in the individual. In some embodiments, the state of single cells is the activation level of one or more activatable elements, e.g., proteins such as phosphoproteins, in the cells. Quantitative analysis, as described herein, is performed, in order to determine the status of the mantle cell lymphoma in the individual. In some embodiments, a treatment decision is made based at least in part on the determination of the status of mantle cell lymphoma using the methods described herein; such treatment decision may include no treatment, treatment with a previously-used treatment, modification of treatment, or use of a new treatment.

In some embodiments, the number of cells associated with mantle cell lymphoma may be determinative when the number of cells is fewer than 10⁻³ to 10⁻⁴ cells. For example, the presence of 1×10⁻³, 2×10⁻³, 3×10⁻³, 4×10⁻³, 5×10⁻³, 6×10⁻³, 7×10⁻³, 8×10⁻³, 9×10⁻³, 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ cells associated with mantle cell lymphoma may be indicative of an individual's status. In some embodiments, the number of cells associated with mantle cell lymphoma may be determinative when the number of cells is higher than 10⁻² to 10⁻⁴ cells. For example, the presence of 1×10⁻², 2×10⁻², 3×10⁻², 4×10⁻², 5×10⁻², 6×10⁻², 7×10⁻², 8×10⁻², 9×10⁻², 1×10⁻³, 2×10⁻³, 3×10⁻³, 4×10⁻³, 5×10⁻³, 6×10⁻³, 7×10⁻³, 8×10⁻³, 9×10⁻³ or 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ cells associated with mantle cell lymphoma may be indicative of an individual's status. In some embodiments, the number of cells associated with mantle cell lymphoma may be determinative when it is correlated with a predetermined clinical parameter. For example in determining the probability of relapse in mantle cell lymphoma patients, patients that have a favorable cytogenetic subtype would have relapses if they have number of cells associated with mantle cell lymphoma higher than for example 10⁻², whereas patients with adverse cytogenetic subtype (e.g., t(11;14) IgH/CCND1 (IgH/BCL1)) would have relapses if they have number of cells associated with mantle cell lymphoma higher than for example 10⁻⁴.

In some embodiments of the invention, the hematologic disease that is evaluated by the methods of the invention is multiple myeloma. Thus, in some embodiments the invention provides methods for diagnosing multiple myeloma, determining a method of treatment for multiple myeloma, determining a prognosis for multiple myeloma, or determining response to treatment for multiple myeloma in an individual, using the methods described herein. In some embodiments, the individual has been previously diagnosed with multiple myeloma and is undergoing or has undergone treatment for multiple myeloma. One or more blood samples are taken from the individual; in some embodiments a series of blood samples are taken from the individual over time. The samples may be taken before, during, or after treatment, or some combination thereof. In some embodiments, the samples are taken before, during, and after treatment. Additional samples or other diagnostic markers, as are known in the art, may also be used in addition to the blood samples to determine the status of the individual; e.g., bone marrow samples may be taken, and/or blood cells may examined for well-established markers of multiple myeloma. In some embodiments, the samples or portions of the samples are treated with a modulator, and the state of single cells is determined, from which a determination is made as to the status of the multiple myeloma in the individual. In some embodiments, the activation level of single cells is the activation level of one or more activatable elements, e.g., proteins such as phosphoproteins, in the cells. Quantitative analysis, as described herein, is performed, in order to determine the status of the multiple myeloma in the individual. In some embodiments, a treatment decision is made based at least in part on the determination of the status of multiple myeloma using the methods described herein; such treatment decision may include no treatment, treatment with a previously-used treatment, modification of treatment, or use of a new treatment.

In some embodiments, the number of cells associated with multiple myeloma may be determinative when the number of cells is fewer than 10⁻³ to 10⁻⁴ cells. For example, the presence of 1×10⁻³, 2×10⁻³, 3×10⁻³, 4×10⁻³, 5×10⁻³, 6×10⁻³, 7×10⁻³, 8×10⁻³, 9×10⁻³, 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ cells associated with multiple myeloma may be indicative of an individual's status. In some embodiments, the number of cells associated with multiple myeloma may be determinative when the number of cells is higher than 10⁻² to 10⁻⁴ cells. For example, the presence of 1×10⁻², 2×10⁻², 3×10⁻², 4×10⁻², 5×10⁻², 6×10⁻², 7×10⁻², 8×10⁻², 9×10⁻², 1×10⁻³, 2×10⁻³, 3×10⁻³, 4×10⁻³, 5×10⁻³, 6×10⁻³, 7×10⁻³, 8×10⁻³, 9×10⁻³ or 1×10⁻⁴, 2×10⁻⁴, 3×10⁻⁴, 4×10⁻⁴, 5×10⁻⁴, 6×10⁻⁴, 7×10⁻⁴, 8×10⁻⁴, or 9×10⁻⁴ cells associated with multiple myeloma may be indicative of an individual's status. In some embodiments, the number of cells associated with multiple myeloma may be determinative when it is correlated with a predetermined clinical parameter. For example in determining the probability of relapse in multiple myeloma patients, patients with specific cell surface proteins or having high levels of somatic hypermutations would have relapses if they have number of cells associated with multiple myeloma higher than for example 10⁻², whereas patients with different cell surface proteins or low levels of somatic hypermutations would have relapses if they have number of cells associated with multiple myeloma higher than for example 10⁻⁴.

In some embodiments of the invention, disease that is evaluated by the methods of the invention is a solid tumor. Thus, in some embodiments the invention provides methods for diagnosing solid tumors, determining a method of treatment for solid tumors, determining the prognosis of a patient with solid tumors, or determining response to treatment of solid tumors in an individual, using the methods described herein. In some embodiments, the individual has been previously diagnosed with a solid tumor and has undergone treatment for a solid tumor. One or more samples are taken from the individual; in some embodiments a series of samples are taken from the individual over time. The samples may be taken before, during, or after treatment, or some combination thereof. In some embodiments, the samples are taken before, during, and after treatment. Samples may be blood samples, lymph node samples, other appropriate samples (dependent on the solid tumor type), or a combination of sample types. Additional samples or other diagnostic markers, as are known in the art, may also be used in addition to the samples analyzed for the state of individual cells. In some embodiments, the samples or portions of the samples are treated with a modulator, and the state of single cells is determined, from which a determination is made as to the status of the solid tumor in the individual. In some embodiments, the state of single cells is the activation level of one or more activatable elements, e.g., proteins such as phosphoproteins, in the cells. Quantitative analysis, as described herein, is performed, in order to determine the status of the solid tumor in the individual. In some embodiments, a treatment decision is made based at least in part on the determination of the status of the solid tumor using the methods described herein; such treatment decision may include no treatment, treatment with a previously-used treatment, modification of treatment, or use of a new treatment. The solid tumor may be any solid tumor amenable to sampling for direct or indirect analysis; solid tumors include but are not limited to head and neck cancer including brain, thyroid cancer, breast cancer, lung cancer, mesothelioma, germ cell tumors, ovarian cancer, liver cancer, gastric carcinoma, colon cancer, prostate cancer, pancreatic cancer, melanoma, bladder cancer, renal cancer, prostate cancer, testicular cancer, cervical cancer, endometrial cancer, myosarcoma, leiomyosarcoma and other soft tissue sarcomas, osteosarcoma, Ewing's sarcoma, retinoblastoma, rhabdomyosarcoma, Wilm's tumor, and neuroblastoma.

Once the status of an individual (e.g., health status) is determined, an appropriate therapeutic action can be taken. The appropriate therapeutic action can take many forms: in the case of cancer, surgery, transplantation, or the administration of a physical, chemical, or biological agent, or combinations thereof. For some individuals, the appropriate action is to initiate a new therapy either in addition to the current therapy or in place of it. For others, a new therapy is not indicated, but instead, the existing therapy should be continued, perhaps in a modified form such as escalating the dosage of a medication. In still other individuals, the existing course of therapy should be shortened, while in others it should be lengthened. In some individuals, the appropriate action is to stop the existing therapy without initiating another form of therapy. In some individuals, the appropriate action is to start supportive care.

In some instances, the appropriate therapy is surgery, of which, numerous forms are known including excisional surgery, cryosurgery, or laser surgery. Surgery can be performed for preventative, curative, or palliative goals. If a predefined class is associated with an elevated risk of developing an organ or tissue specific disease such as breast, colon, or ovarian cancer, prophylactic surgery can be performed to remove the organ or tissue.

In other instances, the appropriate therapy is transplantation. Transplantation includes the transplantation of whole or partial organs, tissues or stem cells from allogenic, autologous, syngenic or xenogenic origin. Stem cells can be derived from peripheral blood, umbilical cord, embryos, bone marrow or other organs and tissue.

In some instances, the appropriate therapy is radiation also known as radiotherapy. Radiation is either electromagnetic or particulate and can be administered by external beam, brachytherapy, or by the administration of radioactive substances including elements, nucleotides, drugs, radiolabeled peptides or radiolabeled antibodies.

In still other instances, the appropriate therapy is the administration of a chemical agent or drug. Such agents comprise a diverse group and can be categorized in numerous ways including by function, chemical structure, or cellular or molecular target.

In one embodiment, the appropriate therapy is the administration of a chemical agent that is a chemotherapy agent used to treat malignancies. Chemotherapeutic agents include, but are not limited to, alkylating agents such as thiotepa and cyclophosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylolomelamine; acetogenins (especially bullatacin and bullatacinone); a camptothecin (including synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, foremustine, lomustine, nimustine, ranimustine; antibiotics such as the enediyne antibiotics (e.g. calicheamicin); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubincin (Adramycin™) (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as demopterin, methotrexate, pteropterin, trimetrexate; purine analogs such as fludarabine, 6-mercaptopurine, thiamiprine, thioguanine; pyrimidine analogues such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replinisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; PSK™; razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethane; vindesine; dacarbazine; mannomustine; mitobronitol; mitolactol; pipobroman; gacytosine; arabinoside (“Ara-C”); cyclophosphamide; thiopeta; taxoids, e.g. paclitaxel (TAXOL™) and docetaxel (TAXOTERE™); chlorambucil; gemcitabine (Gemzar™); 6-thioguanine; mercaptopurine; methotrexate; platinum analogs such as cisplatin and carboplatin; vinblastine; platinum; etoposide (VP-16); ifosfamide; mitroxantrone; vancristine; vinorelbine (Navelbine™); novantrone; teniposide; edatrexate; daunomycin; aminopterin; xeoloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. See Haskell et al, Cancer Treatment, 5^{th} Ed., W.B. Saunders and Co., 2001.

Also included in the definition of “chemotherapeutic agent” are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston™); inhibitors of the enzyme aromatase, which regulates estrogen production in the adrenal glands, such as, for example, 4(5)-imidazoles, aminoglutethimide, megestrol acetate (Megace™), exemestane, formestane, fadrozole, vorozole (Rivisor™), letrozole (Femara™), and anastrozole (Arimidex™); and anti-androgens such as flutamide, nilutamide, bicalutamide, leuprolide, and goserelin; and pharmaceutically acceptable salts, acids or derivatives of any of the above.

In another embodiment, the appropriate therapy is the administration of a chemical agent that is a targeted therapy drug. For the treatment of malignancies, targeted therapeutics include, but are not limited to imatinib mesylate (Gleevec™, also known as STI-571; gefitinib (Iressa™, also known as ZD1839), erlotinib; bortezomib (Velcade™); and oblimersen (Genasense™).

In a further embodiment, the appropriate therapy is the administration of a biological agent comprising native and engineered antibodies including antibodies conjugated to drugs and toxins, antisense oligonucleotides, RNA interference oligonucleotides, peptides, hormones, cytokines, biological response modifiers, vaccines, growth factors, natural products, and ex-vivo expanded tumor-infiltrating lymphocytes.

Biological agents comprise native or engineered antibodies, including antibodies conjugated to drugs and toxins, antisense oligonucleotides, RNA interference oligonucleotides, peptides, hormones, cytokines, biological response modifiers, vaccines, growth factors, natural products, and ex-vivo expanded tumor-infiltrating lymphocytes.

An example of an antibody useful for treating breast cancer is trastuzumab. This antibody recognizes a member of the human epidermal growth factor receptor (HER) family of transmembrane tyrosine kinases HER2/neu (ErbB2).

The determination of the appropriate therapy for an individual may also require assessing one or more other individual characteristics including physical characteristics, clinical status, previous treatment characteristics, and biochemical/molecular markers. Individual characteristics may further comprise patient's past medical history, family medical history, patient's social history, as well as any current medical history termed “review of systems.”

Physical characteristics include an individual's gender; current age; age at the time of disease presentation; age at the time of treatment. Clinical status includes clinical stage of disease, performance status, blood cell count; bone marrow reserves. Factors from previous treatments that can be considered include type of previous therapies, number of previous therapies, response to previous therapy or therapies and time from last treatment. Biochemical and molecular markers include those that serve to define known patient response or outcome to a given therapy. Also included are markers of drug metabolism phenotypes such as cytochrome p450 isoforms.

Determination of response to treatment may comprise the assessment of other factors such as whether there was complete or partial resolution of symptoms, normalization of clinical parameters such as cell counts, or blood chemistry, a reduction in pain or other subjective measurements, or a reduction in pain medication, transfusions, oxygen or other supportive requirements.

### EXAMPLES

### Example 1

### Identification of Subpopulations of Bone Marrow Cells from Normal Individuals and MDS Patients

### Objectives and Study Design:

The objectives of the study were to determine whether cyropreserved samples can be used to characterize MDS and to determine whether a distinct subpopulation of nucleated red blood cells (nRBCs) can be identified in MDS patients. This study was also to design a modulator and staining panel for characterizing responses of MDS patient cell populations including myeloblasts, monocytes, lymphocytes and nRBCs at different developmental stages in response to different stimuli including EPO, IFNγ, FLT3, SCF, and PMA. The modulator and staining panel is shown in Table 1 below.

TABLE 1

| Priority | Modulator | Stain |
|---|---|---|
| 1 | Surface | Erythroid Precursor: CD71, |
| | Phenotype | CD235ab |
| 2 | Surface | Stem Cell: CD117, CD38 |
| | Phenotype | |
| 3 | Surface | CD45 Isoforms: CD45RA, |
| | Phenotype | CD45RO, CD45RB |
| 4 | Surface | Autoimmune: CD3, CD4, CD8 |
| | Phenotype | |
| 5 | Unstim | STAT1/3/5 |
| 6 | EPO | STAT1/3/5 |
| 7 | EPO + G-CSF | STAT1/3/5 |
| 8 | G-CSF | STAT1/3/5 |
| 9 | IL-3 | STAT1/3/5 |
| 10 | IFN-g | STAT1/3/5 |
| 11 | Unstim | Erk, S6, Akt |
| 12 | SCF | Erk, S6, Akt |
| 13 | FLT3L | Erk, S6, Akt |
| 14 | PMA | Erk, S6, Akt |
| 15 | SDF-1a | Erk, S6, Akt |
| 16 | Unstim | Chk2, cleaved PARP |
| 17 | Etoposide | Chk2, cleaved PARP |
| 18 | Unstim | Caspase 8, cleaved PARP |
| 19 | Etoposide | Caspase 8, cleaved PARP |
| 20 | Unstim | NFkB, p38, Erk |
| 21 | LPS | NFkB, p38, Erk |
| 22 | TNF-a | NFkB, p38, Erk |
| 23 | EPO | STAT1/3/5 |
| 24 | EPO + G-CSF | STAT1/3/5 |
| 25 | IL-3 | STAT1/3/5 |
| 26 | IFN-g | STAT1/3/5 |
| 27 | SDF-1a | Erk, S6, Akt |

In this study, there were five MDS patient samples (01-05) and five normal samples (06-10). The clinical information on these 10 samples is summarized in Table 2.

TABLE 2

| | Classi- | | | | | |
|---|---|---|---|---|---|---|
| Sample | fication | Age | Gender | Ethnicity | WBC | BM Blast |
| Sample 01 | RA | 56 | M | White | 3 | 1% |
| Sample 02 | RAEB | 74 | F | Af. American | 8 | 10% |
| Sample 03 | RAEB | 54 | M | White | 4.7 | 14% |
| Sample 04 | RA | 57 | M | White | 3.5 | 2% |
| Sample 05 | RARS | 74 | M | White | 3.1 | 0% |
| Sample 06 | — | 41 | F | — | — | — |
| Sample 07 | — | 23 | M | — | — | — |
| Sample 08 | — | 24 | M | — | — | — |
| Sample 09 | — | 45 | F | — | — | — |
| Sample 10 | — | 31 | M | — | — | — |

### Materials and Methods

The present illustrative example represents how to analyze cells in one embodiment of the present invention. There are several steps in the process, such as the step where a modulator is added, the staining step and the flow cytometry step. The stimulation step of the phospho-flow procedure can start with vials of frozen cells and end with cells fixed and permeabilized in methanol. Then the cells can be incubated with an antibody directed to a particular protein of interest and then analyzed using a flow cytometer. A protocol similar to the following is used to analyze AML cells from patient samples.

The materials used in this invention include thawing medium which comprises PBS-CMF+10% FBS+2 mM EDTA; 70 um Cell Strainer (BD); anti-CD45 antibody conjugated to Alexa 700 (Invitrogen) used at 1 ul per sample; propidium iodide (PI) solution (Sigma 10 ml, 1 mg/ml) used at 1 ug/ml; RPMI+1% FBS medium; media A comprising RPMI+1% FBS+1× Penn/Strep; Live/Dead Reagent, Amine Aqua (Invitrogen); 2 ml, 96-Deep Well, U-bottom polypropylene plates (Nunc); 300 ul 96-Channel Extended-Length D.A.R.T. tips for Hydra (Matrix); Phosphate Buffered Saline (PBS) (MediaTech); 16% Paraformaldehyde (Electron Microscopy Sciences); 100% Methanol (EMD) stored at −20° C.; Transtar 96 dispensing apparatus (Costar); Transtar 96 Disposable Cartridges (Costar, Polystyrene, Sterile); Transtar reservoir (Costar); and foil plate sealers.

a. Thawing Cell and Live/Dead Staining:

Frozen cells are thawed in a 37° C. water bath and gently resuspended in the vial and transferred to the 15 mL conical tube. The 15 mL tube is centrifuged at 930 RPM (200×g) for 8 minutes at room temperature. The supernatant is aspirated and the pellet is gently resuspended in 1 mL media A. The cell suspension is filtered through a 70 um cell strainer into a new 15 mL tube. The cell strainer is rinsed with 1 mL media A and another 12 ml of media A into the 15 mL tube. The cells are mixed into an even suspension. A 20 μL aliquot is immediately removed into a 96-well plate containing 180 μL PBS+4% FBS+CD45 Alexa 700+PI to determine cell count and viability post spin. After the determination, the 15 mL tubes are centrifuged at 930 RPM (200×g) for 8 minutes at room temperature. The supernatant is aspirated and the cell pellet is gently resuspended in 4 mL PBS+4 μL Amine Aqua and incubated for 15 min in a 37° C. incubator. 10 mL RPMI+1% FBS is added to the cell suspension and the tube is inverted to mix the cells. The 15 mL tubes are centrifuged at 930 RPM (200×g) for 8 minutes at room temperature. The cells are resuspended in Media A at the desired cell concentration (1.25×10⁶/mL). For samples with low numbers of cells (<18.5×10⁶), the cells are resuspended in up to 15 mL media. For samples with high numbers of cells (>18.5×10⁶), the volume is raised to 10 mL with media A and the desired volume is transferred to a new 15 mL tube, and the cell concentration is adjusted to 1.25×10⁶ cells/ml. 1.6 mL of the above cell suspension (concentration at 1.25×10⁶ cells/ml) is transferred into wells of a multi-well plate. From this plate, 80 ul is dispensed into each well of a subsequent plate. The plates are covered with a lid (Nunc) and placed in a 37° C. incubator for 2 hours to rest.

b. Addition to a Modulator to the Cells

A concentration for each modulator that is five folds more (5×) than the final concentration is prepared using Media A as diluent. 5× stimuli are arrayed into wells of a standard 96 well v-bottom plate that correspond to the wells on the plate with cells to be stimulated.

Preparation of fixative: Stock vial contains 16% paraformaldehyde which is diluted with PBS to a concentration that is 1.5×. The stock vial is placed in a 37° C. water bath.

Adding the modulator: The cell plate(s) are taken out of the incubator and placed in a 37° C. water bath next to the pipette apparatus. The cell plate is taken from the water bath and gently swirled to resuspend any settled cells. With pipettor, the stimulant is dispensed into the cell plate and vortexed at “7” for 5 seconds. The deep well plate is put back into the water bath.

Adding Fixative: 200 μl of the fixative solution (final concentration at 1.6%) is dispensed into wells and then mixed on the titer plate shaker on high for 5 seconds. The plate is covered with foil sealer and incubated in a 37° C. water bath for 10 minutes. The plate is spun for 6 minutes at 2000 rpm at room temperature. The cells are aspirated using a 96 well plate aspirator (VP Scientific). The plate is vortexed to resuspend cell pellets in the residual volume. The pellet is ensured to be dispersed before the Methanol step (see cell permeabilization) or clumping will occur.

Cell Permeabilization: Permeability agent, for example methanol, is added slowly and while the plate is vortexing. To do this, the cell plate is placed on titer plate shaker and made sure it is secure. The plate is set to shake using the highest setting. A pipetter is used to add 0.6 mls of 100% methanol to the plate wells. The plate(s) are put on ice until this step has been completed for all plates. Plates are covered with a foil seal using the plate roller to achieve a tight fit. At this stage the plates may be stored at −80° C.

c. Staining Protocol

Reagents for staining include FACS/Stain Buffer-PBS+0.1% Bovine serum albumen (BSA)+0.05% Sodium Azide; Diluted Bead Mix-1 mL FACS buffer+1 drop anti-mouse Ig Beads+1 drop negative control beads. The general protocol for staining cells is as follows, although numerous variations on the protocol may be used for staining cells:

Cells are thawed if frozen. Cells are pelleted at 2000 rpm 5 minutes. Supernatant is aspirated with vacuum aspirator. Plate is vortexed on a “plate vortex” for 5-10 seconds. Cells are washed with 1 mL FACS buffer. Repeat the spin, aspirate and vortex steps as above. 50 mL of FACS/stain buffer with the desired, previously optimized, antibody cocktail is added to two rows of cells at a time and agitate the plate. The plate is covered and incubated in a shaker for 30 minutes at room temperature (RT). During this incubation, the compensation plate is prepared. For the compensation plate, in a standard 96 well V-bottom plate, 20 mL of “diluted bead mix” is added per well. Each well gets 5 μL of 1 fluorophor conjugated control IgG (examples: Alexa488, PE, Pac Blue, Aqua, Alexa647, Alexa700). For the Aqua well, add 200 μL of Aqua−/+ cells. Incubate the plate for 10 minutes at RT. Wash by adding 200 μL FACS/stain buffer, centrifuge at 2000 rpm for 5 minutes, and remove supernatant. Repeat the washing step and resuspend the cells/beads in 200 μL FACS/stain buffer and transfer to a U-bottom 96 well plate. After 30 min, 1 mL FACS/stain buffer is added and the plate is incubated on a plate shaker for 5 minutes at room temperature. Centrifuge, aspirate and vortex cells as described above. 1 mL FACS/stain buffer is added to the plate and the plate is covered and incubated on a plate shaker for 5 minutes at room temperature. Repeat the above two steps and resuspend the cells in 75 μl FACS/stain buffer. The cells are analyzed using a flow cytometer, such as a LSRII (Becton Disckinson). All wells are selected and Loader Settings are described below: Flow Rate: 2 uL/sec; Sample Volume: 40 uL; Mix volume: 40 uL; Mixing Speed: 250 uL/sec; # Mixes: 5; Wash Volume: 800 uL; STANDARD MODE. When a plate has completed, a Batch analysis is performed to ensure no clogging.

d. Gating Protocol

Data acquired from the flow cytometer are analyzed with Flowjo software (Treestar, Inc). The Flow cytometry data is first gated on single cells (to exclude doublets) using Forward Scatter Characteristics Area and Height (FSC-A, FSC-H). Single cells are gated on live cells by excluding dead cells that stain positive with an amine reactive viability dye (Aqua-Invitrogen). Live, single cells are then gated for subpopulations using antibodies that recognize surface markers as follows: CD45++, CD33− for lymphocytes, CD45++, CD33++ for monocytes+granulocytes and CD45+, CD33+ for leukemic blasts. Signaling, determined by the antibodies that interact with intracellular signaling molecules, in these subpopulation gates that select for “lymphs”, “monos+grans, and “blasts” is analyzed.

The data can then be analyzed using various metrics, such as basal level of a protein or the basal level of phosphorylation in the absence of a stimulant, total phosphorylated protein, or fold change (by comparing the change in phosphorylation in the absence of a stimulant to the level of phosphorylation seen after treatment with a stimulant), on each of the cell populations that are defined by the gates in one or more dimensions. These metrics are then organized in a database tagged by: the Donor ID, plate identification (ID), well ID, gated population, stain, and modulator. These metrics tabulated from the database are then combined with the clinical data to identify nodes that are correlated with a pre-specified clinical variable (for example; response or non response to therapy) of interest.

### Results:

Staining of CD45 on myeloblasts, mature monocytes and lymphocytes from normal and MDS bone marrow in the presence of PMA shows low variance in CD45 levels among these cell populations, indicating robustness and reproducibility of the CD45 staining (data not shown). For myeblast stimulated with PMA the range for MDS patients was −0.21, −0.31 and the range for normal patients was −0.022, 0.44 and the p value, p-value (Wilcox) and AUC were 0.1584, 0.09524 and 1, respectively. For mature monocytes stimulated with PMA the range for MDS patients was −0.14, −0.085 and the range for normal patients was −0.26, 0.057 and the p value, p-value (wilcox) and AUC were 0.2449, 0.845 and 0.61, respectively. For lymphocytes stimulated with PMA the range for MDS patients was −0.14, −0.072 and the range for normal patients was −0.059, 0.014 and the p value, p-value (wilcox) and AUC were 0.2742, 0.07864 and 1, respectively.

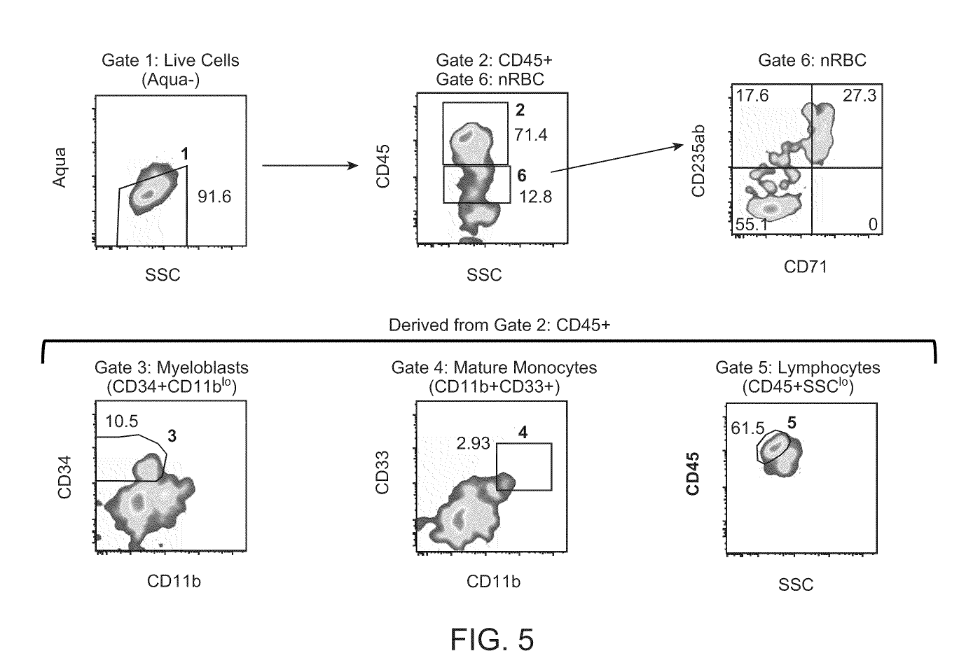

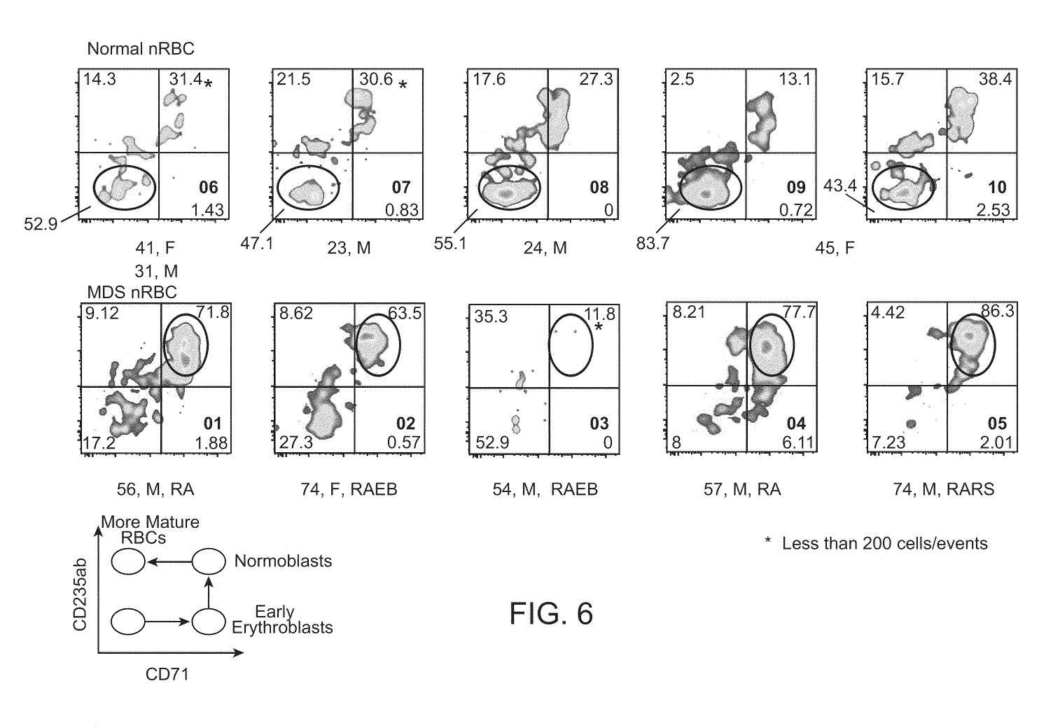

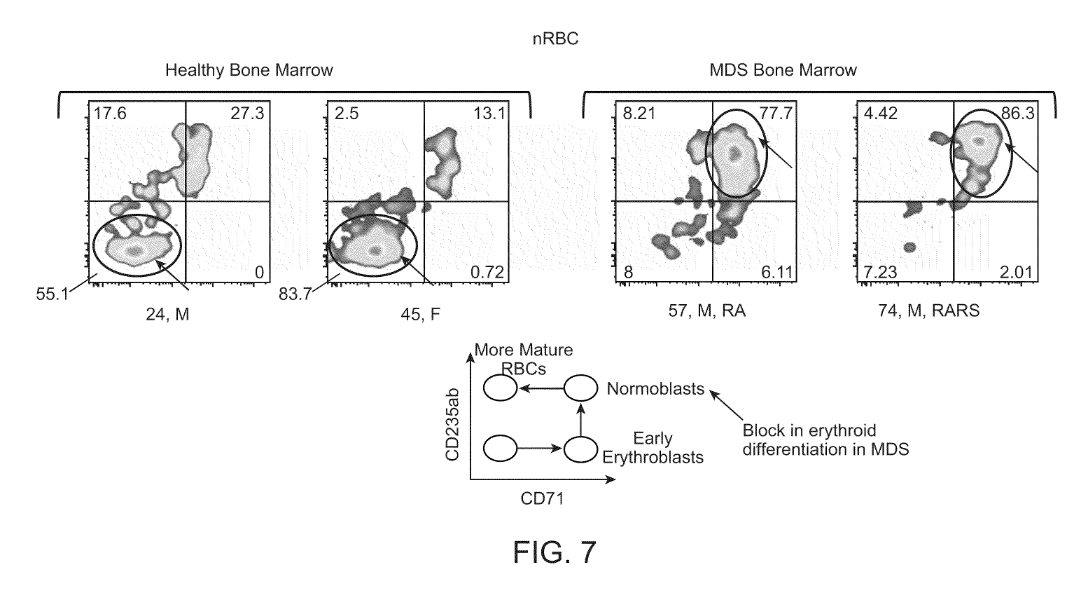

Subpopulations of bone marrow mononuclear cells (BMMCs) from normal and MDS patients were gated and identified by flow cytometry. The bone marrow cells were first gated based on their FSC and SSC profiles, and live cells were identified as Aqua negative in an Aqua vs. SSC plot. Live cells expressing high levels of CD45 were further plotted and gated based on their CD34, CD11b and CD33 expression into CD34+CD11b^{lo} myeloblasts, CD11b+CD33+ mature monocytes, and CD45+SSC^{lo} lymphocytes (FIG. 5). Cells expressing intermediate levels of CD45 were gated as nRBC. nRBCs were further characterized into different developmental stages based on their CD235ab and CD71 expression profiles (FIG. 5). Subpopulations of lymphocytes, for example, CD3+ T cells were identified in normal and MDS bone marrow as CD45+CD3+ (data not shown). Subpopulations of CD3+ T cells, namely, CD4+ and CD8+ T cells in normal and MDS bone marrow were identified based on their surface CD4 and CD8 expression (data not shown). FIG. 6 illustrates identification of nRBCs at different developmental stages, i.e. early erythroblasts, normoblasts, and more mature RBCs based on their CD235ab versus CD71 expression (see, Hoefsloot L H, Lowenberg B et al. Blood, 1997 Mar. 1; 89(5): 1690-700). A comparison of CD235ab versus CD71 expression profiles of nRBCs from normal and MDS bone marrow reveals a higher percentage of CD235+CD71+ normoblasts and a less percentage of CD235ab-CD71− cells in the MDS bone marrow as compared to the normal bone marrow, suggesting a block of erythroid differentiation in MDS. These results suggest that a rare population of CD235+CD71+ may be involved in the pathogenesis of MDS (FIGS. 6 and 7) and can be used for the diagnosis of MDS.

The results show robustness and reproducibility of staining for rare population of cells. Small numbers of subpopulations of bone marrow cells including subsets of T cells and nRBCs from normal individuals and MDS patients can be identified and used to provide clinical information that can be used, for example, in the diagnosis, prognosis, determining progression, predicting response to treatment or choosing a treatment.

### Example 2

### Cellular Responses of Subpopulations of Bone Marrow Cells from Normal Individuals and MDS Patients

nRBCs (identified in Example 1) from normal individuals and MDS patients, were stimulated with various stimuli including EPO, IFNγ, FLT3, SCF, PMA, G-CSF and the combinations thereof. The cell stimulation and staining were carried out according to the detailed protocols described in Example 1.

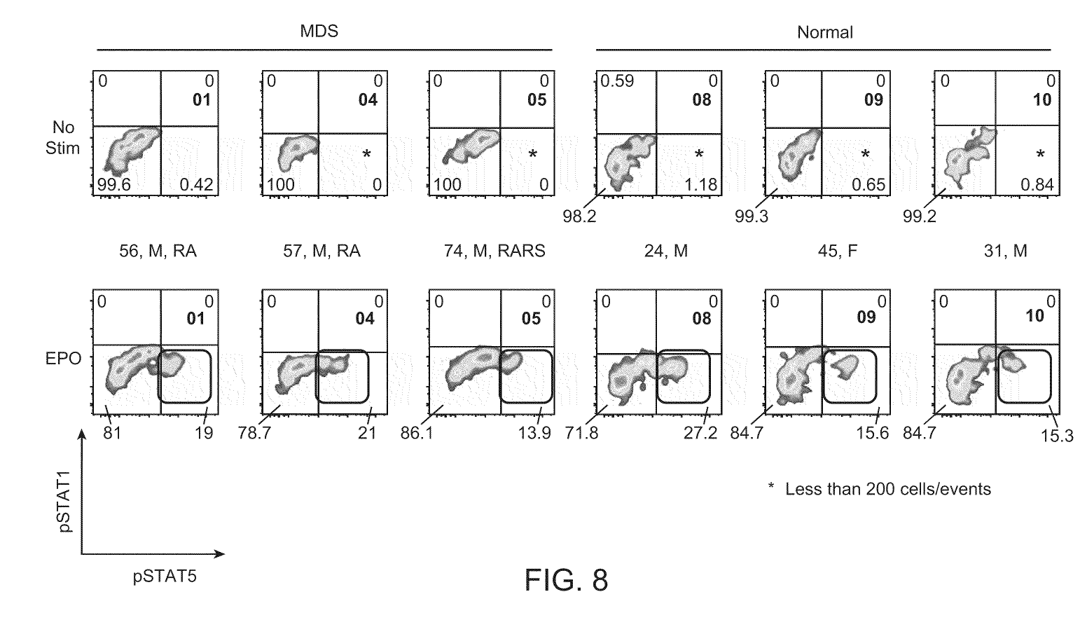

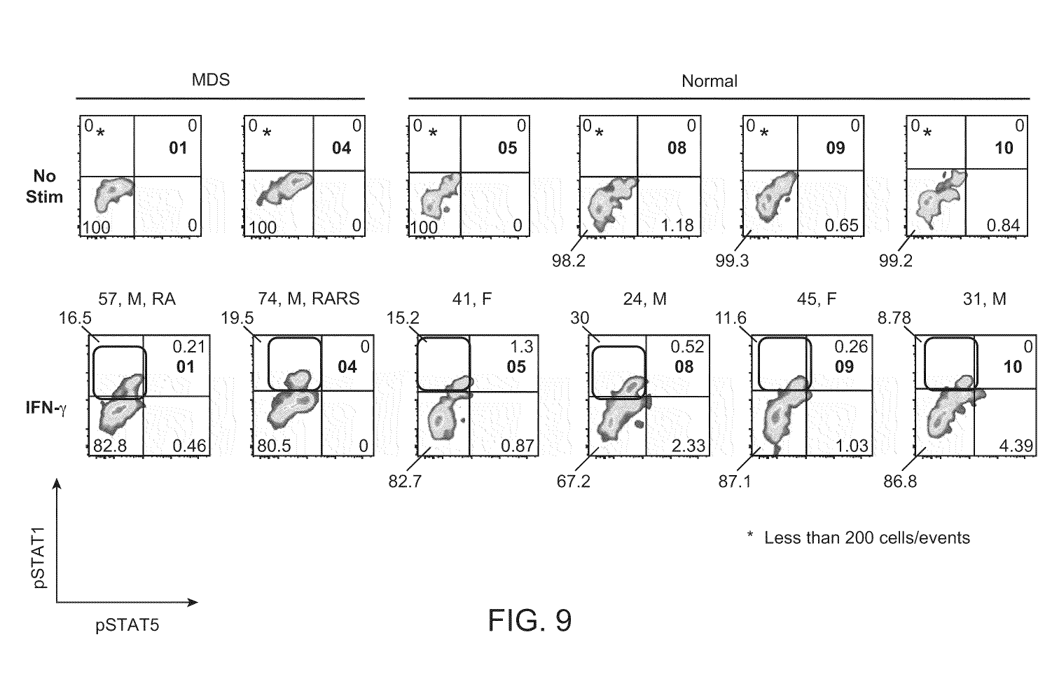

A variety of fluorochrome-conjugated antibodies that recognize cell surface and intracellular markers including CD11b, CD33, CD34, CD45, C-casp8, C-PARP, pAkt, pChk2, perk, pNFkb, p-p38, p-S6, pSTAT1, pSTAT3, and pSTAT5 were incubated with the cells. nRBCs from normal individuals and MDS patients were treated with erythropoietin (EPO) and the EPO-mediated Stat5 and Stat1 phosphorylation was assessed by flow cytometry. As shown in FIG. 8, nRBC subpopulation from MDS patients exhibits Stat5 phosphorylation in response to EPO stimulation. This response in a small population to EPO stimulation identifies a rare cell population. Interestingly, the shapes of the contour plots, for both unstimulated and stimulated samples, are different between MDS and Normal patients. FIG. 9 shows Stat5 and Stat1 phosphorylation in rRBCs from normal and MDS patients in response to interferon gamma (IFNγ) stimulation. A small nRBC subpopulation from MDS patients exhibits Stat1 phosphorylation in response to IFNγ stimulation. These results demonstrate the ability to measure the cellular responses of small numbers of cells present in MDS patients. Thus, the methods described herein can be used to detect a small number of cells, which may be related to a disease such as cancer and can be used for it diagnosis.

### Example 3

### Effects of Therapeutics on Healthy Bone Marrow Cells

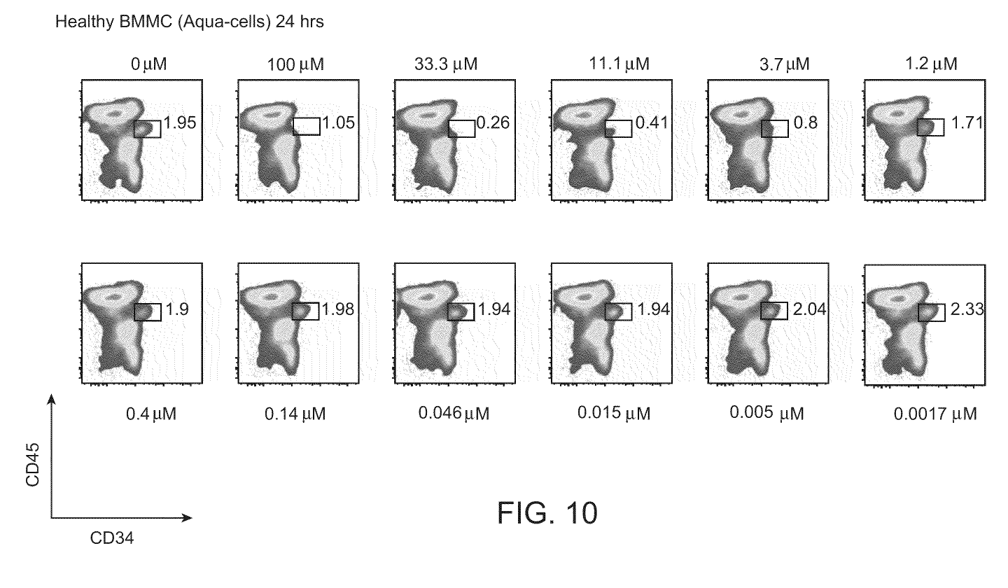

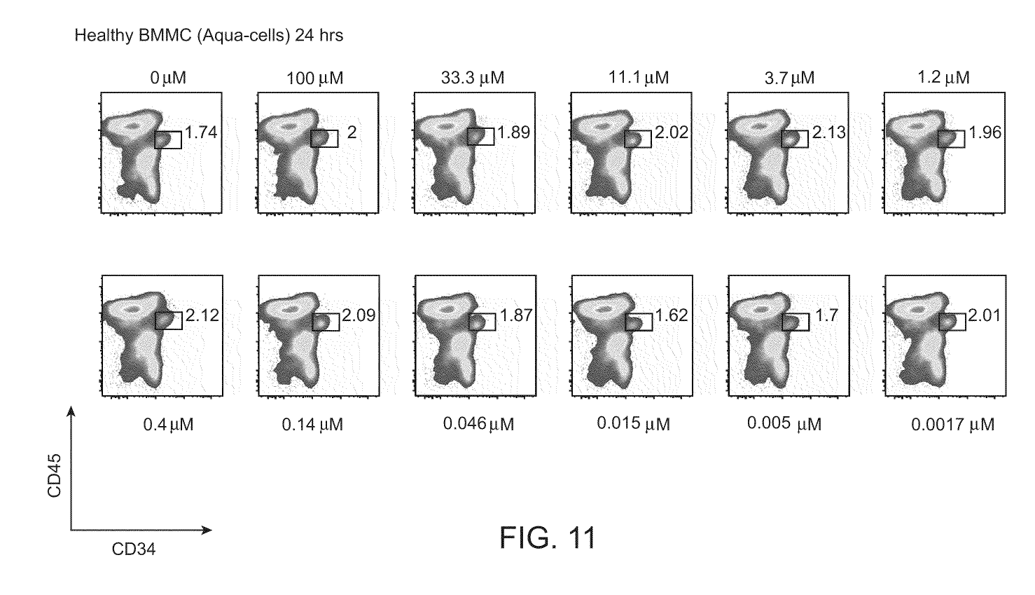

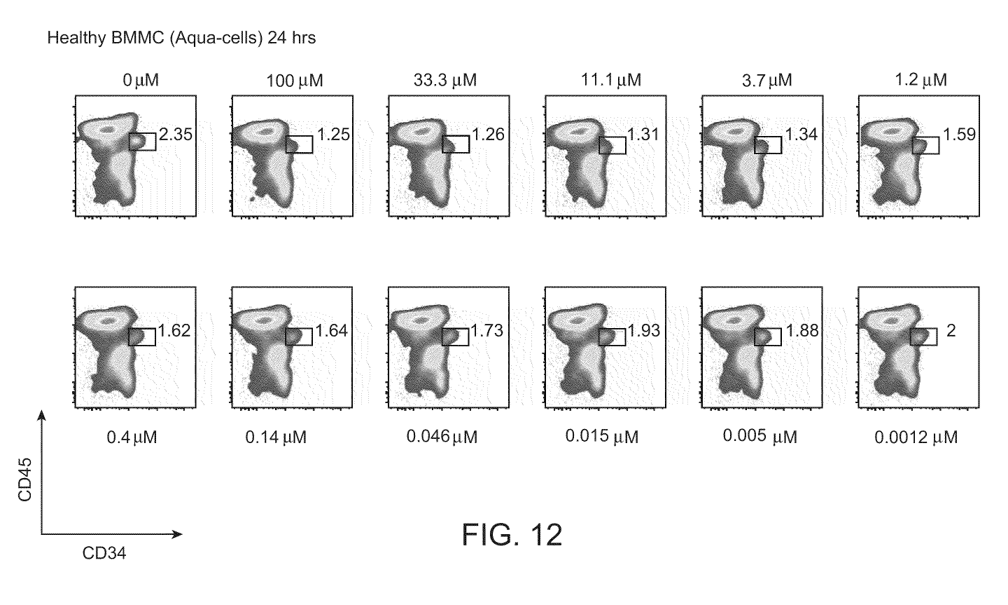

Live healthy bone marrow mononuclear cells (BMMCs) were contacted with several drugs at different concentrations by a 1:3 dilution in the medium, for example, 100 μM, 33.3 μM, 11.1 μM, 3.7 μM, 1.2 μM, 0.4 μM, 0.14 μM, 0.046 μM, 0.015 μM, 0.005 M, or 0.0017 μM of 5-Azacytidine (Vidaza), Decitabine (Dacogen), Vorinostat (Zolina) and DMSO. CD45 and CD34 expression was assessed by flow cytometry after 24 hours of stimulation with each drug. The cell stimulation and staining were carried out according to the detailed protocols described in Example 1. The CD45 versus CD34 expression profiles of healthy BMMCs exposed to 5-Azacytidine (Vidaza), Decitabine (Dacogen), Vorinostat (Zolinza), or DMSO are shown in FIGS. 10-12, respectively. 5-Azacytidine (Vidaza) and Decitabine (Dacogen) are hypomethylating agents. The results shown that 5-Azacytidine (Vidaza) results in a dose-dependent loss of a rare population of CD34+ myeloblast cells (FIG. 10). In contrast, Decitabine (Dacogen), a drug in the same molecular class as Vidaza, does not affect the viability of the rare populationCD34+ myeloblast cells (FIG. 11). Vorinostat (Zolinza), a histone deacetylase (HDAC) inhibitor, shows selective loss of rare population of CD34+ myeloblast cells in a dose-dependent fashion (FIG. 12).

The results show that the methods described herein enable the measurement of drug responses in small populations of cells.

### Example 4

### CD45RA/RO/RB Expression Profiles of Subpopulations of Bone Marrow Cells from Normal Individuals and MDS Patients

Cells from normal and MDS bone marrows were gated based on their CD45 and SSC expression profile as described above. CD45RA, CD45RO and CD45RB expression on nRBCs was assessed by flow cytometry. CD45RO, CD45RA, and CD45RB are isoforms of CD45. Each CD45 isoforms is distinguished from one another isoform depending on the type of exon the CD45 has or the exons the CD45 does not have. The CD45RA isoform contains the A exon only and the CD45RB has the B exon only whereas the CD45RO has none of the exons: A, B, or C. Altered expression of CD45 isoforms on hematopoietic cells, particularly lymphocytes, has been associated with various diseases.

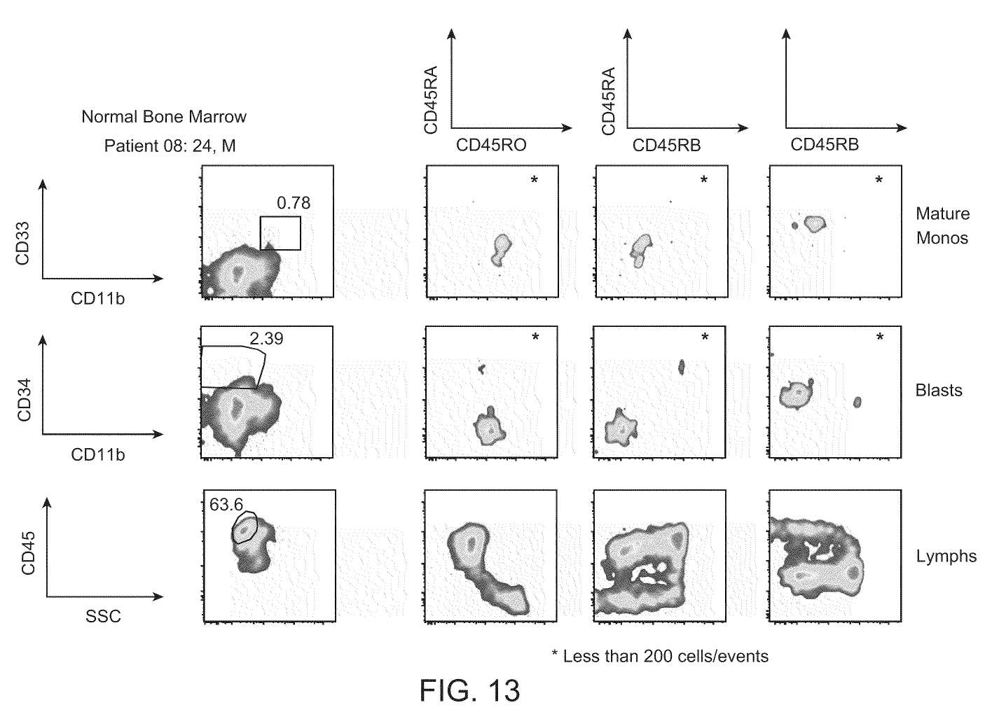

FIGS. 12 and 13 shows CD45RA/RO/RB expression profiles of mature monocytes, myeloblasts and lymphocytes from normal and MDS bone marrows. Mature monocytes in the bone marrow were gated as CD33^{hi}CD11b^{hi}. Myeloblasts were gated as CD34⁺CD11b^{lo}, and lymphocytes were gated based on their CD45 and SSC expression profiles. CD45RA, CD45RO and CD45RB expressions on monocytes, myeloblasts and lymphocytes were assessed by flow cytometry. The results show differences in CD45RA/RO/RB levels between normal individuals and MDS patients among different subpopulations of mature monos, blast and lymphocytes. CD45 isoform expression, thus, identifies unique rare cells populations in MDS patients

In summary, the study of the present invention suggests that cryopreserved MDS patient samples can be used to examine myeloblasts, erythroid precursors, monocytes, and lymphocytes in terms of their surface molecule expression, such as CD45RA/RO/RB expression. The results show that small populations of cells, which may be involved in a disease condition such as cancer, can be detected and used for the diagnosis of MDS.

### Example 5

### A Small Population of Cells Responsive to Stem Cell Factor (SCF) Exist at Diagnosis and Expand During Disease Progression

The objective if this study is to identify cells in a diagnosis sample and compare the results with a sample taken at a later time point from the same patient that will predict patient outcome. To achieve this objective myeloid populations were gated in the samples. Two dimensional (2D) plots are created for signaling analysis while three dimensional plots (3D) are created for identifying cell lineage subsets. Gates are drawn on cells with increase signaling to then back-gate to identify phenotype of cells as determined by cell surface markers. This method allows for the identification of differences in signaling between diagnosis and later time-point samples. The gates delineating cells with increased signaling are applied to myeloid populations from independent studies with AML samples.

Samples from AML patients were taken at diagnosis and at different time points after treatment. Cells in the samples were stimulated and stained according to the detailed protocols described in Example 1. Different populations of cells in the AML patients were compared at the time of diagnostics and at the time of relapse.

a. Gating of Flow Cytometry Data to Identify Live Cells and the Lymphoid and Myeloid Subpopulations:

Flow cytometry data can be analyzed using several commercially available software programs including FACSDiva™, FlowJo, and Winlist™. The initial gate is set on a two-parameter plot of forward light scatter (FSC) versus side light scatter (SSC) to gate on “all cells” and eliminate debris and some dead cells from the analysis. A second gate is set on the “live cells” using a two-parameter plot of Amine Aqua (a dye that brightly stains dead cells, commercially available from Invitrogen) versus SSC to exclude dead cells from the analysis. Subsequent gates are be set using antibodies that recognize cell surface markers and in so doing define cell sub-sets within the entire population. A third gate is set to separate lymphocytes from all myeloid cells (acute myeloid leukemia cells reside in the myeloid gate). This is done using a two-parameter plot of CD45 (a cell surface antigen found on all white blood cells) versus SSC. The lymphocytes are identified by their characteristic high CD45 expression and low SSC. The myeloid population typically has lower CD45 expression and a higher SSC signal allowing these different populations to be discriminated. The gated region containing the entire myeloid population is also referred to as the P1 gate.

b. Phenotypic Gating to Identify Subpopulations of Acute Myeloid Leukemia Cells:

The antibodies used to identify subpopulations of AML blast cells are CD34, CD33, and CD11b. The CD34⁺ CD11b⁻ blast population represents the most immature phenotype of AML blast cells. This population is gated on CD34 high and CD11b negative cells using a two-parameter plot of CD34 versus CD11b. The CD33 and CD11b antigens are used to identify AML blast cells at different stages of monocytic differentiation. All cells that fall outside of the CD34⁺CD11b⁻ gate described above (called “Not CD34+”) are used to generate a two-parameter plot of CD33 versus CD11b. The CD33⁺ CD 11b^{hi} myeloid population represents the most differentiated monocytic phenotype. The CD33⁺CD11b^{intermediate} and CD33⁺CD11b^{lo} populations represent less differentiated monocytic phenotypes.

c. Back Gating to Identify the Phenotype of G-CSF and SCF Responsive Cells:

A two-parameter or 3-parameter (3-D) plot was generated from the P1 gate (all myeloid cells). For G-CSF stimulation, the signaling responses measured were p-Stat1, p-Stat3, and p-Stat5. The 3-D plot of p-Stat1 vs. p-Stat3 vs. pStat5 was generated in Spotfire. The two-parameter plots were generated in FlowJo.

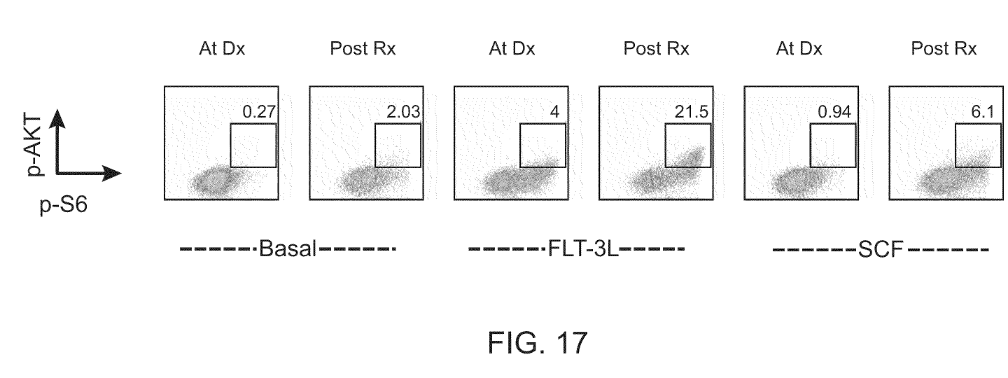

The data files for the unstimulated control sample and the G-CSF treated sample were overlaid for comparison. In the results discussed below, the paired patient samples at diagnosis (MDL-7) and at relapse (MDL-8) are shown. On the 3-D plot, the G-CSF responsive population was readily visible as a p-Stat5 positive population (See FIG. 17). A gate was set on the p-Stat5 positive population and was used to back gate onto a 3-D plot of CD34 vs. CD33 vs. CD11b generated from the P1 gate. The data shows that the G-CSF responsive cells were found mainly in the CD33⁺ CD11b⁻ population and that in the relapse sample there was an increase in G-CSF responsive cells within the CD33⁺CD11b⁻ population (4% at diagnosis compared to 27% at relapse). Analysis of G-CSF responsive populations in healthy bone marrow showed that the responding cells are mainly CD34⁺.

d. Results

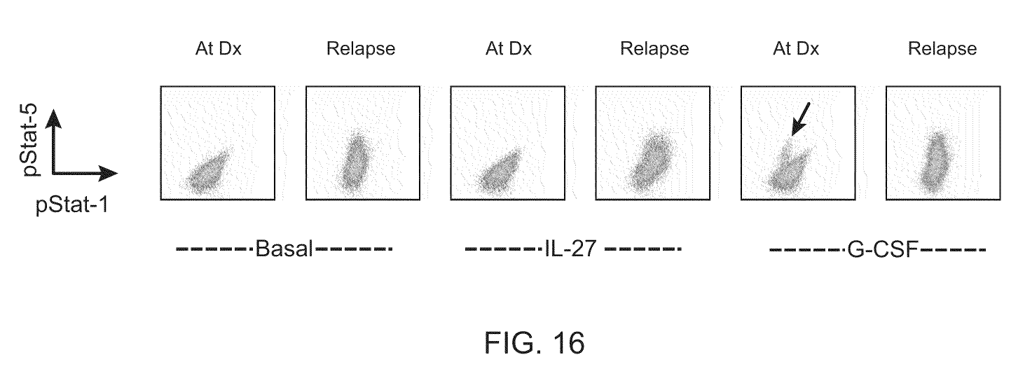

In this CR relapse patient two samples are available for analysis. One sample was taken at the time of diagnosis and the second was taken about 4 months later when the patient relapsed. The samples were measured for their basal phosphorylated Stat-5 (p-Stat5) and Stat-1 (p-Stat1) and the phosphorylated levels in response to IL-27 and G-CSF (FIG. 16). FIG. 16 shows an example of a bone marrow sample at diagnosis and relapse from a 34 year old patient whose response was CR Relapse with M2 AML and Flt3 ITD+. Comparison of the two samples revealed more p-Stat5 and p-Stat1 in the samples taken at relapse. FIG. 16 shows that at diagnostics there is a small sample that show levels of p-Stat-5 in response to G-CSF. This population is increased at relapse (See arrow in FIG. 16).

In addition, the samples were evaluated for their basal levels of phosphorylated Akt (p-Akt) and ribosomal S6 protein (p-S6) (FIG. 17). FIG. 17 shows an example of results in a bone marrow sample at diagnosis and post induction treatment from a 68 year old patient who was a refractory to induction therapy and therefore classified as a non-responder (NR) and with M5 AML and Flt3R wild-type. Comparison of the two samples revealed more p-Akt and p-S6 in the samples taken at relapse. The two samples were also treated with stem cell factor (SCF) and FLT3L and the signaling response was evaluated by determining the levels of p-Akt and p-S6. In the sample taken at diagnosis, a small population of cells showed a response to SCF and the dots in the gate show cells with an increase in p-Akt and p-S6 (See FIG. 17). However, there was a far greater increase in the SCF-mediated increase in p-Akt and p-S6 in the sample taken at relapse. Back-gating revealed the phenotype of the responding cell population which was identified as a myeloid cell sub-set defined by CD33+, CD11b−, CD34−. Table 3 describes the phenotypes of the SCF-responsive cells

TABLE 3

| | Phenotype of SCF |
|---|---|
| Subject | Responsive Cell Subsets |
| AML Patient 1 | CD34+, CD33−, CD11b− |
| AML Patient 2 | CD34+, CD33+, CD11b− |
| AML Patient 3 | CD34−, CD33+, CD11b− |
| Healthy | CD34+, CD33−, CD11b− |

These responding cells did not respond as robustly to FLT3 ligand stimulation. However, it is clear that there is a small population of SCF responsive (double positive) cells in the sample at diagnosis. This finding was seen in all the patients with matched (DX and Relapse) samples (n=3).

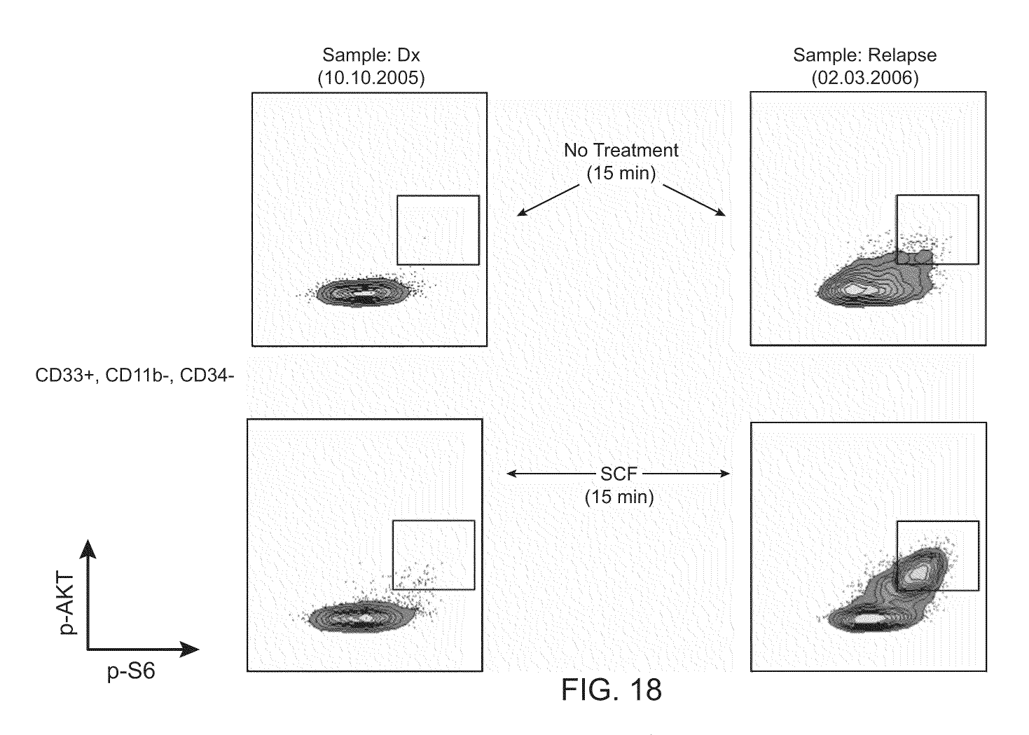

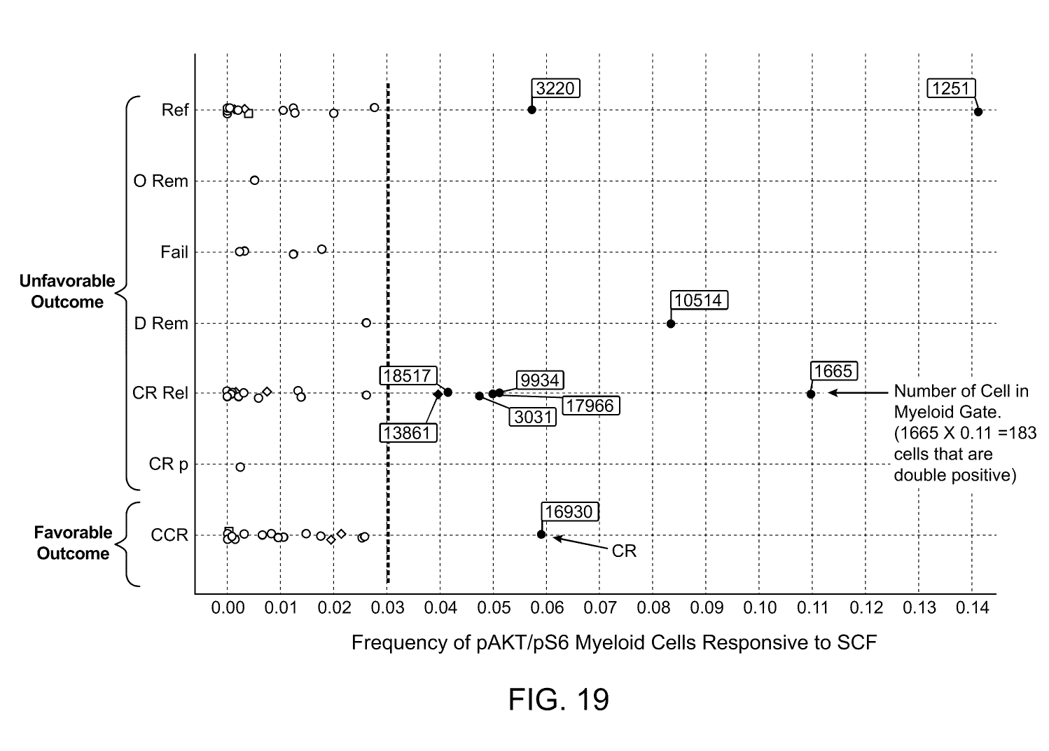

In order to predict whether the presence of a small population of SCF responsive (p-Akt/p-S6) double positive population at diagnosis could predict outcome, a gate that delineated the double positive population was applied to a set of historical phosphoflow data from a set of AML samples taken at diagnosis and evaluated for SCF signaling in an independent study (FIG. 18). FIG. 18 shows results from the bone marrow of a CR relapse 34 year old patient with M2 AML and Flt3 ITD+. FIG. 19 depicts the results for the SCF responsive (p-Akt/p-S6) double positive population in the set of AML patients. The results show that 9/10 patients with an SCF responding double positive cell frequency of >3% relapsed within two years (FIG. 19). Only one patient in which there was an SCF-responding double population had a complete clinical response (CCR). Furthermore, only a small number of cells were necessary to stratify these patients. As shown in slide 5, in one particular patient, 183 double positive cells were captured.

To summarize, in this small patient subset 3/3 evaluated patients had the double positive SCF responding cells. As mentioned above, in an independent study with a larger number of AML patient samples taken at diagnosis, 9/10 patients with an SCF responding double positive cell frequency of >3% relapsed within two years (FIG. 19). Notably, not all of the patients that had a poor outcome exhibited this SCF response. The cell surface phenotype of the double positives are generally negative for CD11b surface protein, but can be either CD34 positive, CD33 positive, or a combination of both (see Table 3). This contrasts with healthy bone marrow in which the SCF responsive cells are restricted to the CD34+ subset.

When the analysis using the same gate was performed in peripheral blood mononuclear cells (PBMCs) from AML patients, a trend similar to the bone marrow data was seen (data not shown). Since SCF-responsive cells are not present in the blood circulation of healthy subjects, PBMCs or whole peripheral blood may be a preferred source of cells for an assay that measures the SCF responsive double positives since background “assay noise” could be avoided. It would be predicted that any SCF signaling would emanate from the diseased cells.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of predicting a change in a health status in an individual from a first state to a second state comprising:
(a) determining the presence of a first and second class of cells in a sample from said individual said presence being determined by a method comprising determining an activation level of an intracellular activatable element in single cells from said sample;
(b) classifying said single cells into said first and second class, wherein at least one class is classified based on said activation level;
(c) calculating a ratio of said first and second class of cells; and
(d) predicting a change in a health status in said individual from a first state to a second state when said ratio exceeds a threshold number.

2. The method of claim 1, wherein said classes are predefined classes.

3. The method of claim 1, wherein said threshold number is a predetermined threshold number, wherein said predetermined threshold number has been associated with said second state.

4. The method of claim 1, wherein said second state is the location of an individual on a continuum that comprises normal, pre-pathological, and pathological states.

5. The method of claim 4, wherein said continuum is a continuum wherein the pathological state is an immunologic, malignant, or proliferative disorder or a combination thereof.

6. The method of claim 5, wherein the malignant disorder is a solid tumor or a hematologic malignancy.

7. The method of claim 5, wherein said malignant disorder is non-B cell lineage derived.

8. The method of claim 7, wherein said non-B cell lineage derived malignant disorder is selected from the group consisting of Acute myeloid leukemia (AML), Chronic Myeloid Leukemia (CML), non-B cell Acute lymphocytic leukemia (ALL), non-B cell lymphomas, myelodysplastic disorders, myeloproliferative disorders, myelofibroses, polycythemias, thrombocythemias, and non-B cell atypical immune lymphoproliferations.

9. The method of claim 5, wherein said malignant disorder is a B cell or B cell lineage derived disorder.

10. The method of claim 9, wherein said malignant disorder is a B-Cell or B cell lineage derived disorder selected from the group consisting of Chronic Lymphocytic Leukemia (CLL), B cell lymphocyte lineage leukemia, B cell lymphocyte lineage lymphoma, Multiple Myeloma, and plasma cell disorders

11. The method of claim 1, further comprising predicting a response to a treatment for a pre-pathological or pathological condition, or a response to treatment for a pre-pathological or pathological condition.

12. The method of claim 1, wherein the activation levels of a plurality of intracellular activatable elements in single cells is determined.

13. The method of claim 1, wherein said plurality of cells obtained from said individual is first exposed to a modulator before determining said activation level of said activatable element.

14. The method of claim 13, wherein said modulator is an activator or an inhibitor.

15. The method of claim 14, wherein said modulator is a growth factor, cytokine, adhesion molecule modulator, hormone, small molecule, polynucleotide, antibody, natural compound, lactone, chemotherapeutic agent, immune modulator, carbohydrate, protease, ion, reactive oxygen species, or radiation.

16. The method of claim 1 wherein the sample is a blood sample, a biopsy sample or a surgical sample.

17. The method of claim 1, wherein the class is a class of cells wherein one or more activation levels of the cells are different when compared to normal control values, or when compared to previous determinations made in a series of samples from said individual.

18. The method of claim 1, wherein said predicting a change in said health status in said individual is performed on a plurality of samples from said individual.

19. The method of claim 18, wherein said plurality of samples comprises samples from different locations in the individual, samples taken at different times from the individual, samples treated in different ways prior to determining the activation level, or a combination thereof.

20. The method of claim 19, wherein the method further comprises determining the rate of change of said ratio.

21. The method of claim 20, wherein, said rate of change is expressed as the doubling time of said cells.

22. The method of claim 1, further comprising determining an appropriate course of treatment for said individual based on said status of the individual.

23. The method of claim 22, wherein said appropriate course of treatment comprises watchful waiting, supportive care, initiating a therapy, not initiating a therapy, stopping, shortening, prolonging, or modifying an existing therapy, adding an additional therapy to existing therapy, or combinations of the foregoing.

24. The method of claim 22, wherein said therapy is selected from the group consisting of surgical excision, transplantation, or the administration of a physical, chemical, or biological agent, or combinations thereof.

25. The method of claim 1, wherein one or more characteristics of the individual is determined, and the change in health status in the individual is determined based on both the ratio and the one or more characteristics of the individual.

26. The method of claim 22 wherein said determining of an appropriate course of treatment is also based on one or more characteristics of the individual.

27. The method of claim 25, wherein said one or more characteristics is physical characteristics, clinical status, treatment characteristics, biochemical/molecular markers or a combination thereof.

28. The method of claim 1, wherein said activation level is based on the activation state selected from the group consisting of extracellular protease exposure, novel hetero-oligomer formation, glycosylation state, phosphorylation state, acetylation state, methylation state, biotinylation state, glutamylation state, glycylation state, hydroxylation state, isomerization state, prenylation state, myristoylation state, lipoylation state, phosphopantetheinylation state, sulfation state, ISGylation state, nitrosylation state, palmitoylation state, SUMOylation state, ubiquitination state, neddylation state, citrullination state, deamidation state, disulfide bond formation state, proteolytic cleavage state, translocation state, changes in protein turnover, multi-protein complex state, oxidation state, multi-lipid complex, and biochemical changes in cell membrane.

29. The method of claim 28, wherein said activation state is a phosphorylation state.

30. The method of claim 1, wherein said classifying of said single cells further comprises determining cell size, cell granularity, the presence or absence of one or more cell surface markers, the presence or absence of one or more intracellular markers, or combination thereof.

31. The method of claim 30, wherein said cell surface markers and said intracellular markers are independently selected from the group consisting of proteins, carbohydrates, lipids, nucleic acids and metabolites.

32. The method of claim 30, wherein said determining of the presence or absence of one or more cell surface markers or intracellular markers comprises determining the presence or absence of an epitope in both activated and non-activated forms of said one or more cell surface markers or intracellular markers.

33. The method of claim 30, wherein said activatable element is selected from the group consisting of proteins, carbohydrates, lipids, nucleic acids and metabolites.

34. The method of claim 33, wherein said activatable element is a protein.

35. The method of claim 34, wherein said protein is a protein subject to phosphorylation and/or dephosphorylation.

36. The method of claim 34, wherein said protein is selected from the group consisting of kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation.

37. The method of claim 34, wherein said protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Wee1, Casein kinases, PDK1, SGK1, SGK2, SGK3, Akt1, Akt2, Akt3, p90Rsks, p70S6Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3a, GSK3p, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon γ, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bcl-2, Mcl-1, Bcl-XL, Bcl-w, Bcl-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, p14Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pin1 prolyl isomerase, topoisomerases, deacetylases, Histone deacetylases, sirtuins, histone acetylases, CBP/P300 family, MYST family, ATF2, DNA methyl transferases, Histone H3K4 demethylases, H3K27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Sp1, Egr-1, T-bet, β-catenin, HIFs, FOXOs, E2Fs, SRFs, TCFs, Egr-1,1-catenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors.

38. The method of claim 1, wherein said activation level is determined by a process comprising the binding of a binding element which is specific to a particular activation state of the particular activatable element.

39. The method of claim 38, wherein said binding element comprises an antibody.

40. The method of claim 33, wherein said activatable element is responsive to a change in metabolic state, temperature, local ion concentration, or expression of a heterologous protein.

41. The method of claim 1, wherein the step of finding the activation level comprises the use of flow cytometry, immunofluorescence, confocal microscopy, immunohistochemistry, immunoelectronmicroscopy, nucleic acid amplification, gene array, protein array, mass spectrometry, patch clamp, 2-dimensional gel electrophoresis, differential display gel electrophoresis, microsphere-based multiplex protein assays, ELISA, and label-free cellular assays to determine the activation level of one or more intracellular activatable element in single cells.

42. The method of claim 1 wherein said threshold number expressed as a percentage is about 30%.

43. The method of claim 1 wherein said threshold number expressed as a percentage is about 5%.

44. The method of claim 1 wherein said threshold number expressed as cell frequency is about 10⁻⁴.
